SEQUENCE LISTING (34 sequences)

SEQ ID NO: 1 - protein, 119 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSS

SEQ ID NO: 2 - protein, 106 aa, synthetic construct [sequence omitted]

SEQ ID NO: 3 - protein, 240 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSS

SEQ ID NO: 4 - protein, 240 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 5 - protein, 240 aa, synthetic construct
QVQLLESGAELARPGASVKMSCKASGYTFTRYTMHWVKQRPGQGLEWIGYINPSRGYTNYNQKFKDKATLTTDKSSSTAYMQLSSLTSEDSAVYYCAGYYDDHYCLDYWGQGTLVTVSSGGGGSGGGGSGGGGSDIVMTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIK

SEQ ID NO: 6 - protein, 8 aa, synthetic construct
NRVRQGYS

SEQ ID NO: 7 - protein, 30 aa, synthetic construct
IYIWAPLAGTCGVLLLSLVITLYCKRGRKK

SEQ ID NO: 8 - protein, 112 aa, synthetic construct
DIVMTQTPLSLPVSLGDQASISCRSSQILVDSNENTYLHWYLQKPGQSPKLLIYKVSNRVSGVPDRFSGSGSGTDFTLKISRVEAEDLGVYFCSQNTHVPPTFGGGTKLEIK

SEQ ID NO: 9 - protein, 118 aa, synthetic construct
EVQLQQSGPELVKPGASVKMSCKASGYTFTNYVLHWVKQNSGQGLEWIGFIDPYNDGAQYNEKFKGKATLTSDKSSSTVYMEFSSLTSEDSAVYYCTREGGIGGFAYWGQGTLVTVSA

SEQ ID NO: 10 - protein, 111 aa, synthetic construct
DIVLTQSPPSLAVSLGQRATISCRASKSVSTSGYRFLHWYQQKPGQAPKLLIYLASNLESGVPARFSGSGSGTDFTLNIHPVEEEDAATYYCQHSREFPWTFGGGTKLEIK

SEQ ID NO: 11 - protein, 119 aa, synthetic construct
EVQLQQSGAEVVRSGASVKLTCTVSGFNIKDHYMHWVKQRPEQGLEWIGWIDPENGDTEYDPKFQGKATMTADISSNTAYLQLNSLTSEETAVYYCNALDSSGYGFAYWGQGTLVTVSA

SEQ ID NO: 12 - protein, 607 aa, synthetic construct
MLSYQDKVGAFYKDNARANSSKLSLVTEEQGGRRPPYLLFVLLILLVGIMTLLAITGVRFHQVSTSNMEFSRLLKEDMEKSEAVHHQVIDVLTPLFKIIGDEVGLQLPQKLNEIKQFILQKTNFFNPNREFDFRDLHWCINPPSKIKVNFTNYCDTIGIKKSIASAANPILLSALSGGRGDIFPPYRCSGATTSVGRVFPLSVSLSMSLISKTSEITSMLTAISDGVYGKTYLLVPDYIEGEFDTQKIRVFEIGFIKRWLNNMPLLQTTNYMVLPENSKAKVCTIAVGELTLASLCVDESTVLLYHDSNGSQDGILVVTLGIFGATPMDQVEEVIPVAHPSVEKIHITNHRGFIKDSIATWMVPALVSEKQEEQKNCLESACQRKTYPMCNQTSWEPFGGGQLPSYGRLTLPLDPSIDLQLNISFTYGPVILNGDGMDYYESPLSDSGWLTIPPRNGTVLGLINKASRGDQFTVIPHVLTFAPRESSGNCYLPIQTSQIMDKDVLTESNLVVLPTQNFRYVIATYDISRGDHAIVYYVYDPIRTISYTHPFRLTTKGRPDFLRIECFVWDDDLWCHQFYRFEADITNSTTSVENLVRIRFSCNRSKP

SEQ ID NO: 13 - protein, 662 aa, synthetic construct
MHNKIPKGSKTPTPTQQDPPPQHSIKSAETKTSQARYNITSAQQSTRHGPRTSDRPVHYIMNRTRSCKQASYRSDNIPAHGDHEGIIHHTPESVSQGVRSRFKRRQSNATSSGSQCTWLVLWCIGVASLFLCSKAQIHWNNLSTIGIIGTDSVHYKIMTRPSHQYLVIKLMPNVSLIDNCTKAELGEYEKLLSSVLEPINQSLTLMTKNVKPLQSVGSGRRQRRFAGVVLAGAALGVATAAQITAGIALHQSNLNAQAIQSLRTSLEQSNKAIEEIREATQETIIAVQGVQDYVNNELVPAMQHMSCELVGQRLGLKLLRYYTELLSIFGPSLRDPISAEISIQALSYALGGEIHKILEKLGYSGNDMIAILESRGIKTKITHVDIPGKLIILSISYPTLSEVKGVIVHRLEAVSYNIGSQEWYTTVPRYVATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGVTIQVGGRQYPDMVYESKVALGPAISLERLDVGTNLGNALKKLDDAKILIDSSNQILETVRRSSFNFGSLLSVPILICTALALLFLIYCCKRRYQQTLKQHIKVDPTFKPDLTGTSKSYVRSL

SEQ ID NO: 14 - protein, 135 aa, synthetic construct
MHNKIPKGSKTPTPTQQDPPPQHSIKSAETKTSQARYNITSAQQSTRHGPRTSDRPVHYIMNRTRSCKQASYRSDNIPAHGDHEGIIHHTPESVSQGVRSRFKRRQSNATSSGSQCTWLVLWCIGVASLFLCSKA

SEQ ID NO: 15 - protein, 22 aa, synthetic construct
MLLLVTSLLLCELPHPAFLLIP

SEQ ID NO: 16 - protein, 45 aa, synthetic construct
TTTPAPRPPTPAPTIASQPLSLRPEACRPAAGGAVHTRGLDFACD

SEQ ID NO: 17 - protein, 21 aa, synthetic construct
MALPVTALLLPLALLLHAARP

SEQ ID NO: 18 - protein, 550 aa, synthetic construct
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 19 - protein, 5 aa, synthetic construct
CCRGR

SEQ ID NO: 20 - protein, 12 aa, synthetic construct
ALLFLIYCCKRR

SEQ ID NO: 21 - protein, 16 aa, synthetic construct
MLSLVTEEQGGRRPPY

SEQ ID NO: 22 - protein, 7 aa, synthetic construct
MGRRPPY

SEQ ID NO: 23 - protein, 42 aa, synthetic construct
KRGRKKLLYIFKQPFMRPVQTTQEEDGCSCRFPEEEEGGCEL

SEQ ID NO: 24 - protein, 41 aa, synthetic construct
RSKRSRLLHSDYMNMTPRRPGPTRKHYQPYAPPRDFAAYRS

SEQ ID NO: 25 - protein, 245 aa, synthetic construct
DIVMTQTPLSLPVSLGDQASISCRSSQILVDSNENTYLHWYLQKPGQSPKLLIYKVSNRVSGVPDRFSGSGSGTDFTLKISRVEAEDLGVYFCSQNTHVPPTFGGGTKLEIKGGGGSGGGGSGGGGSEVQLQQSGPELVKPGASVKMSCKASGYTFTNYVLHWVKQNSGQGLEWIGFIDPYNDGAQYNEKFKGKATLTSDKSSSTVYMEFSSLTSEDSAVYYCTREGGIGGFAYWGQGTLVTVSA

SEQ ID NO: 26 - protein, 245 aa, synthetic construct
DIVLTQSPPSLAVSLGQRATISCRASKSVSTSGYRFLHWYQQKPGQAPKLLIYLASNLESGVPARFSGSGSGTDFTLNIHPVEEEDAATYYCQHSREFPWTFGGGTKLEIKGGGGSGGGGSGGGGSEVQLQQSGAEVVRSGASVKLTCTVSGFNIKDHYMHWVKQRPEQGLEWIGWIDPENGDTEYDPKFQGKATMTADISSNTAYLQLNSLTSEETAVYYCNALDSSGYGFAYWGQGTLVTVSA

SEQ ID NO: 27 - protein, 27 aa, synthetic construct
FWVLVVVGGVLACYSLLVTVAFIIFWV

SEQ ID NO: 28 - protein, 49 aa, synthetic construct
AVGQDTQEVIVVPHSLPFKVVVISAILALVVLTIISLIILIMLWQKKPR

SEQ ID NO: 29 - protein, 21 aa, synthetic construct
METDTLLLWVLLLWVPGSTGD

SEQ ID NO: 30 - protein, 16 aa, synthetic construct
MKCLLYLAFLFIGVNC

SEQ ID NO: 31 - protein, 28 aa, synthetic construct
MGAGATGRAMDGPRLLLLLLLGVSLGGA

SEQ ID NO: 32 - protein, 22 aa, synthetic construct
LIPVYCSILAAVVVGLVAYIAF

SEQ ID NO: 33 - protein, 14 aa, synthetic construct
SSIASFFFIIGLII

SEQ ID NO: 34 - protein, 119 aa, synthetic construct [sequence omitted]

(19) United States
(12) Patent Application Publication
(10) Pub. No.: US 2026/0286392 A1
(43) Pub. Date: Sep. 24, 2026

(54) PSEUDOTYPED RETROVIRAL VECTOR PARTICLE WITH ANTI-CD3 DISPLAY

(71) Applicant: Miltenyi Biotec B.V. & Co. KG, Bergisch Gladbach (DE)

(72) Inventors: Nora Winter, Bergisch Gladbach (DE); Nicole Cordes-Paulitz, Bergisch Gladbach (DE); Thomas Schaser, Bergisch Gladbach (DE)

(73) Assignee: Miltenyi Biotec B.V. & Co. KG, Bergisch Gladbach (DE)

(21) Appl. No.: 19/478,767

(22) PCT Filed: Apr. 26, 2024
(86) PCT No.: PCT/EP2024/061576

§ 371 Date: Oct. 24, 2025

(30) Foreign Application Priority Data
Apr. 27, 2023 (EP) 23170224.2

(51) Int. Cl. C12N 15/86; C07K 14/005; C07K 16/28

(52) U.S. Cl. CPC C12N 15/86; C07K 14/005; C07K 16/2809; C07K 2317/622; C07K 2319/03; C12N 2740/15043; C12N 2760/18422; C12N 2760/20222

(57) ABSTRACT

The present invention provides a pseudotyped retroviral vector particle for activating and transducing T cells, wherein said retroviral vector particle comprises a modulating protein comprising a functional ectodomain comprising an antigen binding domain specific for the antigen CD3, wherein said antigen binding domain specific for the antigen CD3 comprises a humanized and optimized scFV sequence, wherein said retroviral vector particle comprises at least one nucleic acid sequence encoding a transgene, and wherein said retroviral vector particle is a lentiviral or gammaretroviral vector particle. A pharmaceutical composition thereof is also disclosed.

### FIELD OF THE INVENTION

The present invention generally relates to the field of the generation of pseudotyped retroviral vector particles, in particular to the generation of pseudotyped retroviral vector particles with anti-CD3 display.

### BACKGROUND OF THE INVENTION

T cells play a key role in cell-mediated immunity and are therefore an interesting target for adoptive immunotherapy such as chimeric antigen receptor (CAR) T cell immunotherapy. State-of-the-art CAR T manufacturing processes involve isolation of T cells from the human body, polyclonal activation, genetic modification with lentiviral vectors (LV) followed by an expansion phase. This complex process is time and cost intensive, but also results in a high degree of in vitro modification of the T cells. Decreasing the process complexity e.g. by omitting the activation step or by reducing the expansion phase therefore offers the potential to improve the cost efficiency of CAR T manufacture. In addition, reduced ex vivo cultivation and production from a naive phenotype was shown to improve CAR T cell function (Ghassemi et al (2018); Arcangeli et al (2022)). In-situ generation of CAR T cells offers the potential to further reduce the complexity of manufacturing and to completely avoid in vitro manipulation of the T cells. To achieve this goal, efficient methods are required to transduce non-activated T cells and subsequently to induce sufficient proliferation in vivo.

Transduction of non-activated T cells is challenging, as the natural receptor of the state-of-the-art LV pseudotyped with Vesicular Stomatitis Virus glycoproteins (VSV-G LV) is not expressed in quiescent state (Amirache et al (2013)). Consequently, VSV-G LVs cannot bind to T cells—a requirement for successful transduction. In addition, even when LVs can bind to target cells, the efficiency of integration is decreased on non-activated T cells due to activity of intracellular restriction factors (Dai et al (2009)). Receptor-targeted LVs, e.g. pseudotyped with Measles glycoproteins (i.e. MV-LV) or Nipah virus glycoproteins (i.e. NiV-LV) overcome the restriction of target cell binding, since the targeted receptors are also expressed in a quiescent state. However, the transduction efficiency on quiescent T cells with receptor-targeted LVs remains to be low.

Polyclonal activation of T cells enhances both, the expression of required receptors of VSV-G LV, but also the integration efficiency. T cells require simultaneous engagement of the T-cell receptor with the major histocompatibility complex peptide and co-stimulatory molecules on the antigen presenting cells for activation. In vitro this can be achieved by supplementing CD3 and CD28 agonists to the culture, e.g. anti-CD3 and anti-CD28 antibodies. Alternatively, T cells may be activated with anti-CD3 and anti-CD28 antibodies coupled to beads (Kalamasz et al (2004), Li et al (2010)). In WO2016180721A1 an in vitro method for polyclonal stimulation of T cells is presented comprising contacting a population of T cells with a humanized anti-CD3 antibody fragment (a scFV of a mutated Oct3 clone) linked to solid particles. Optimal stimulation of T cells needs additional presentation of an anti-CD28 antibody fragment, linked to particles.

Alternatively, activation of T cells can be induced by retroviral vectors that present activating ligands within their envelope. Maurice et al (2002) presented a technology for displaying the anti-CD3 scFV (Okt3) on the envelope of VSV-G pseudotyped retroviral vectors. They demonstrated both activation of T cells and transduction of non-activated T cells, however proliferation was decreased compared to T cells activated with anti-CD3 an anti-CD28 antibodies. Derdak et al (2006), described the development of activating virus-like particles. Here, they displayed the anti-CD3 scFV (Okt3) on virus-like particles with a MoML V core, by expressing it in the context of a CD5L signal domain and CD14/CD16 transmembrane domain with a GPI linker. They described both successful activation and proliferation of the T cells. However, proliferation was dependent on co-stimulatory signals.

In WO2022/164935A1 a viral particle is disclosed comprising a vector genome comprising a polynucleotide sequence encoding an anti-CD19 chimeric antigen receptor, wherein the viral particle transduces immune cells in vivo, and the particle may comprise a viral envelope that comprises an anti-CD3 single-chain variable fragment exposed on the surface and/or conjugated to the surface of the viral envelope.

In WO2020/106992A1 a multicistronic vector for surface-engineering retroviral particles is disclosed, comprising a polynucleotide operatively linked to a promoter, wherein the polynucleotide encodes a plurality of polypeptides joined by linkers comprising peptides capable of inducing ribosome skipping or self-cleavage, and wherein the plurality of polypeptides comprise a fusion glycoprotein or functional variant thereof and one or more non-viral proteins capable of viral surface display. Said non-viral protein may be specific for human CD3 (anti-CD3 scFV).

In WO2019/200056A2 a retroviral vector particle for activating and efficiently transducing T cells is disclosed, comprising a nucleic acid sequence encoding a small-molecule controllable T-cell/NK-cell activation receptor, the nucleic acid sequence operatively linked to a promoter, wherein the T-cell/NK-cell activation receptor is capable of being activated by a small molecule. Said retroviral vector particle may be a surface-engineered retroviral vector particle comprising a T-cell activation or co-stimulation molecule. Said T-cell activation or co-stimulation molecule may comprise one or more of an anti-CD3 antibody, CD28 ligand, and 41bb ligand.

In WO2018/033726 retroviral vector particles for activating and efficiently transducing T cells is disclosed, comprising a mitogenic T cell activating transmembrane protein which binds to mitogenic tetraspanins. Said retroviral vector may also comprise an additional transmembrane protein which binds CD3.

Alternative technologies for activating T cells with retroviral vector particles is described by Frank et al (2020). Here, NiV-LVs are retargeted to T cells by fusion of CD3-scFVs (clone TR66 and HuM291) to Nipah-G. They demonstrate successful transduction and activation of non-activated T cells. However, both, marker expression and cytokine secretion are reduced compared to state-of-the art T cell activation. Moreover, proliferation was only induced upon co-stimulation with CD28-specific antibodies. Huckaby et al (2019) described activation of T cells using Sindbis-pseudotyped LVs. Here, Sindbis-pseudotyped LV were retargeting a bispecific Fab molecule (a-CD3 and Sindbis E2). However, transduction efficiency on non-activated T cells remained low and activation of T cells was not evaluated.

In WO2021/154839A1 a method of transducing immune cells in a subject in need thereof is disclosed, comprising: a) administering a multispecific antibody to render immune cells in the subject more transducible; and b) administering a vector, optionally a viral vector; wherein the method transduces the immune cells. Said multispecific antibody may comprise anti-CD3. It is well known that retroviruses and also retroviral vectors incorporate host cell proteins or artificial proteins in their envelope. However, since the process of protein incorporation into retroviral envelopes is not well understood, the required protein structure needs to be evaluated. Rasbach et al (2013) described the display of a third envelope protein in measles pseudotyped retroviral vector particles. They displayed an a-Her2 scFV on the viral particle by expressing it in the pDisplay vector system, however reproducibility with alternative scFVs was not shown. Taube et al (2008) describe the expression of antibodies in cells and on viral vectors. Here a scFV with FC region was designed in the context of a IgG Signal and gp41, CD8 or CD28 (short extracellular domain & transmembrane domain). In addition, a gp41 incorporation signal was attached. The influence of the protein architecture on the cell surface expression and viral incorporation was described.

Therefore, there is a need in the art for improved or alternative pseudotyped retroviral vector particles for activation and transduction of T cells that may be used e.g. for in vivo activation and transduction of T cells. All of the described methods require additional technologies, e.g. co-stimulation to induce sufficient expansion of T cells. Therefore, there is a need in the art for improved or alternative pseudotyped retroviral vector particles for activation and transduction of T cells, that also induce sufficient expansion of T cells, while maintaining a favorable T cell phenotype suitable to induce long lasting therapeutic efficacy. This technology will be used to improve the production process of gene modified T cells in vitro and/or for in vivo activation and transduction of T cells. Especially in vivo transduction would benefit from activating LVs that do not require incorporation of co-stimulatory molecules that may induce off-target effects.

### BRIEF DESCRIPTION OF THE INVENTION

The present invention makes use of the discovery that retroviral vector particles such as lentiviral vectors can display T cell activating ligands within their envelope. The inventors surprisingly identified a modulating protein, as disclosed herein, especially advantageous in the field of immunotherapy to combine activation and genetic modification of T cells in one step induced by the same viral particles.

Expression of the modulating protein, comprising SEQ ID NO:1 (VH) and SEQ ID NO:2 (VL) has become manifest for example in SEQ ID NO:3 or SEQ ID NO:4 for the binding to CD3, in HEK293T cells was enhanced compared to the modulating protein, having SEQ ID NO:5 for the binding to CD3. Moreover, the surface expression on HEK293T cells was influenced by the choice of signal peptide, cytoplasmic domain as well as transmembrane domain of the modulating protein. But of most importance is the selection of the transmembrane domain. The mechanisms of protein incorporation into LVs is poorly understood and the level of protein expression on a cell surface does not necessarily correlate with the virion-incorporated amount of the same protein. Hence, it was surprising to find improved incorporation of the modulating protein into the LV comprising SEQ ID NO:1 and SEQ ID NO:2 for the binding to CD3. Surprisingly, it was found that presentation of the modulating protein comprising SEQ ID NO:1 and SEQ ID NO:2 for the binding to CD3 on LV particles enhanced activation of T cells compared to LVs presenting the modulating protein having SEQ ID NO:5 for the binding to CD3.

Activation of T cells is characterized by upregulation of surface markers like CD25 or CD69, as well as induction of proliferation. Surprisingly, by treatment of T cells with LV displaying an a-CD3 scFV comprising SEQ ID NO:1 and SEQ ID NO:2 (e.g. SEQ ID NO:3 or SEQ ID NO: 4), activation markers were up-regulated and efficient proliferation was induced without the need of co-stimulatory signals.

Here, T cell targeted LVs were generated that comprise two targeting ligands, CD4/CD8 and CD3. Targeted LVs based on morbillivirus or henipavirus derived glycoproteins undergo pH-independent binding and fusion for transduction. The agonistic effect of anti-CD3 antibodies induce CD3 crosslinking and subsequent internalization, which was suspected to block efficient cell contact of CD3-targeted LVs. As a result, transduction efficiency with CD3-targeted LVs was low and could only be rescued with blocking agents, i.e. Dasatinib (A. Braun et at, 2022). CD3-targeted morbillivirus pseudotyped retroviral vectors have not been described in the art. Moreover, the inventors found that retargeting of morbillivirus pseudotyped retroviral vectors to CD3 does not result in productive transduction. Hence it was surprising to find that simultaneous transduction and activation was especially efficient with CD4- or CD8-targeting morbillivirus pseudotyped retroviral vectors that were displaying the modulating protein as disclosed herein. Consequently, it was surprising to find comparable or enhanced transduction efficiencies when targeted LVs are used that additionally presented a CD3-binding protein within their envelope.

The pseudotyped retroviral vector particle as disclosed herein is especially well-suited for in vivo transduction of T cells in a subject but also may be used for in vitro transduction of T cells in a cell culture, not requiring additional reagents to activate as it combines activation and genetic modification in one entity.

In a preferred embodiment of the invention the retroviral vector particle for activating and transducing T cells, wherein said retroviral vector particle comprises a modulating protein comprising a functional ectodomain comprising an antigen binding domain specific for the antigen CD3, wherein said antigen binding domain specific for the antigen CD3 comprises SEQ ID NO: 1 and SEQ ID NO:2, comprises a nucleic acid encoding a CAR.

For in vivo application the retroviral vector particle as disclosed herein may be formulated for administration to a subject by, for example, injection.

The retroviral vector particle may be administered only once or multiple times. Such pharmaceutical compositions may be useful for transducing quiescent T cells, with the gene product of a desired protein such as a CAR that, if expressed in the T cell, leads to the prevention or the treatment of a particular medical condition.

### BRIEF DESCRIPTION OF THE DRAWINGS

FIG. 1: Schematic representation of the retroviral vector particles for activating and transducing T cells.

To enable simultaneous activation and transduction of human T cells a modulating protein was designed that integrates into the retroviral envelope.
A Shown are retroviral vector particles of the disclosure, which are pseudotyped with envelope proteins of Canine distemper virus (CDV), Measles virus (MV), Nipah virus (NiV) or Vesicular Stomatitis virus (VSV-G). To enable selective transduction with CDV, MV and NiV-LV, the receptor binding protein (H or G) was mutated to ablate binding to the natural receptor and fused to an scFV specific for the antigen of choice. Besides the viral glycoproteins also the modulating protein is incorporated in the viral envelope.
B Surface-engineered retroviral vector particles with incorporated modulating protein can activate human T cells by binding to the T cell receptor (TCR), while transduction is mediated via the viral glycoproteins that are binding to the designated receptor on the T cells. Treatment of T cells with these retroviral vectors results in increased proliferation, metabolism and activation marker expression.

FIG. 2: Schematic representation of the modulating protein and quantification of surface expression.
A Schematic representation of the construct used to generate surface-engineered retroviral vector particles displaying an a-CD3 scFV. The a-CD3 scFV is expressed under the control of a CMV promoter followed by a signal peptide (SP) to enable translocation to the cell membrane.

The variable domains of the scFV may be linked by a (G₄S)₃ linker and the entire scFV may be flanked by two tags for detection. Different transmembrane and cytoplasmic domains were used to anchor the scFV in the LV envelope.
B HEK293T cells were either left untreated (mock) or were transiently transfected with the plasmids encoding the modulating protein or GFP as positive control (GFP). Surface expression of the modulating protein comprising two different a-CD3 scFV (CD3mut scFV: SEQ ID NO: 4; CD3 scFV: SEQ ID NO: 5) and two different signal peptides (hCSF2R SP: SEQ ID NO 15; CD8 SP: SEQ ID NO 17) was determined by flow cytometry two days post transfection upon staining for a-CD3 scFV expression.

FIG. 3: Production of adapter-based retroviral vector particles and quantification of transfection efficiency.

Second-generation LVs are generated via transient transfection of HEK293T using plasmids encoding the envelope proteins for attachment and fusion, the plasmid encoding the modulating protein (a-CD3 scFV), helper plasmids encoding gag, pol and rev and the plasmid encoding the transgene. Upon harvest of the LV, the transfection efficiency can be determined by quantification of the transgene expression by flow cytometry.
A The schematic representation of the production process of retroviral particles is shown.
B Exemplary data is shown for the surface expression of the viral glycoprotein (CDV H) and the a-CD3 scFV in presence (with a-CD3 display) or absence (without a-CD3 display) of the modulating protein compared to the negative control (untransfected).
C Transfection efficiency quantified after production of GFP-encoding LV is shown for vector particles displaying different modulating proteins or no modulating protein (CDV-LV).

FIG. 4: Functional titer of adapter-based retroviral vector particles.

Retroviral vector particles were produced from transient transfected HEK293T cells. Functional titers were quantified by transducing biotinylated SupT1 cells with serially diluted retroviral vector. Transduction efficiency was analyzed four days post transduction by quantification of marker positive cells. LV titers are represented as transducing units per volume (TU/mL), calculated by the ratio of transduced cells and applied LV volume.
A The titer of concentrated CDV-pseudotyped retroviral vector particles displaying the modulating protein comprising an a-CD3 scFV (CD3mut scFV: SEQ ID NO: 4; CD3 scFV: SEQ ID NO: 5) expressed with indicated signal peptides (hCSF2R SP: SEQ ID NO: 15; IgK SP: SEQ ID NO: 29; VSVG SP: SEQ ID NO: 30, LNGFR SP: SEQ ID NO: 31) and transmembrane domains (PDGFR TM: SEQ ID NO: 28; CD8 TM: SEQ ID NO: 7; CD28 TM: SEQ ID NO: 27; VSVG TM: SEQ ID NO: 33; LNGFR TM: SEQ ID NO: 32) is shown. CDV-pseudotyped retroviral particles (CDV-LV) without display of an a-CD3 scFV was used as positive control.
B The titer of unconcentrated CDV-pseudotyped retroviral vector particles displaying the modulating protein comprising an a-CD3 scFV (CD3mut scFV: SEQ ID NO: 4; CD3 scFV: SEQ ID NO: 5) expressed with indicated signal peptides (hCSF2R SP: SEQ ID NO: 15; CD8 SP: SEQ ID NO: 17) and transmembrane domains (CD8 TM: SEQ ID NO: 7; CD28 TM: SEQ ID NO: 27; VSVG™: SEQ ID NO: 33) is shown. CDV-pseudotyped retroviral particles (CDV-LV) without display of an a-CD3 scFV was used as positive control.

FIG. 5: Incorporation of a-CD3 scFV within the envelope of adapter-based retroviral particles. To confirm incorporation of a-CD3 scFV in functional retroviral particles GFP-encoding CDV-pseudotyped retroviral particles (CDV-LV) were immobilized via a N-terminal tag of the modulating protein in an ELISA plate. Unbound particles were removed by thorough washing with PBS. Subsequently SupT1 were added to the immobilized particles to allow transduction. The transduction efficiency was determined by quantification of GFP-expressing cells by flow cytometry seven days after transduction confirming presence of functional particles. CDV-pseudotyped LV (CDV-LV) without a-CD3 scFV display or wells without treatment of any LV (mock) were used as negative control. Incorporation of a-CD3 scFV in functional retroviral vector particles displaying the modulating protein comprising an a-CD3 scFV (CD3mut scFV: SEQ ID NO: 4; CD3 scFV: SEQ ID NO: 5) expressed with different signal peptides (hCSF2R SP: SEQ ID NO: 15; Igκ SP: SEQ ID NO: 29; VSVG SP: SEQ ID NO: 30) and transmembrane domains (PDGFR TM: SEQ ID NO: 28; CD8 TM: SEQ ID NO: 7; CD28 TM: SEQ ID NO: 27; VSVG TM: SEQ ID NO: 33) are shown. Background transduction levels are indicated by the dotted line.

FIG. 6: Analysis of activation marker expression, proliferation and transduction efficiency upon transduction with adapter-based retroviral particles.

Non-activated cell-trace dye stained CD8+ T cells of two healthy donors were left untreated (mock) or were transduced with GFP-encoding CDV-pseudotyped retroviral vectors (CDV-LV) at a dose of 10 TU/cell in presence of CD8-specific adapter molecules. Retroviral vector particles displaying the modulating protein comprising an a-CD3 scFV (CD3mut scFV: SEQ ID NO: 4; CD3 scFV: SEQ ID NO: 5) expressed with different signal peptides (hCSF2R SP: SEQ ID NO: 15; Igκ SP: SEQ ID NO: 29; VSVG SP: SEQ ID NO: 30; LNGFR SP: SEQ ID NO: 31) and transmembrane domains (PDGFR TM: SEQ ID NO: 28; CD8 TM: SEQ ID NO: 7; CD28 TM: SEQ ID NO: 27; VSVG TM: SEQ ID NO: 33; LNGFR TM: SEQ ID NO: 32). Transduction efficiency was analyzed by quantification of GFP-expressing cells using flow cytometry. Activation was quantified by staining for the activation marker CD25 and subsequent flow cytometry. T cells treated with Transact™ (mock TA) were used as positive control.
A CD25 expression of CD8+ T cells is shown five days after transduction with CDV-LV alone (CDV-LV), in presence of Transact™ (CDV-LV TA) or with CDV-LV containing a-CD3 scFV expressed with different signal peptides and transmembrane domains.
B Exemplary data for the proliferation of CD8+ T cells is shown five days post transduction visualized by a fluorescent cell-trace dye.
C Transduction efficiency of CD8+ T cells five days post transduction is shown.

FIG. 7: Generation of CAR-T cells from non-activated PBMC with adapter-based retroviral particles.

Non-activated PBMC of two healthy donors were left untreated (mock) or were transduced with CD20CAR encoding CDV-pseudotyped retroviral vectors (CDV-LV) at a dose of 2.5 TU/cell in presence or absence of CD8-specific adapter molecules. Retroviral vector particles displaying the modulating protein comprising an a-CD3 scFV (CD3mut scFV: SEQ ID NO: 4; CD3 scFV: SEQ ID NO: 5) expressed with different signal peptides (hCSF2R SP: SEQ ID NO: 15; Igκ SP: SEQ ID NO: 29; VSVG SP: SEQ ID NO: 30) and transmembrane domains (PDGFR TM: SEQ ID NO: 28; CD8 TM: SEQ ID NO: 7; CD28 TM: SEQ ID NO: 27). The integrase inhibitor Raltegravir was used to confirm retroviral gene transfer. Transduction efficiency was analyzed by quantification of marker positive (LNGFR) cells using flow cytometry. In addition, the cell count of viable CD20-expressing target cells (B cells) was determined by flow cytometry.
A Transduction efficiency of CD8+ T cells is shown seven days after transduction with CDV-LV alone (CDV-LV), in the presence of Transact™ (CDV-LV TA) or with CDV-LV containing a-CD3 scFV expressed with different signal peptides and transmembrane domains. PBMC activated with Transact™ (mock TA) were used as control.
B The B cell count seven days post transduction is shown.

FIG. 8: Surface expression of the modulating protein.

HEK293T cells were either left untreated (mock) or were transiently transfected with the plasmids encoding the modulating protein. Surface expression of the modulating protein comprising an a-CD3 scFV (CD3mut scFV: SEQ ID NO: 4; CD3 scFV: SEQ ID NO: 5) expressed with different signal peptides (hCSF2R SP: SEQ ID NO: 15; Igκ SP: SEQ ID NO: 29; VSVG SP: SEQ ID NO: 30; CD8 SP: SEQ ID NO: 17; LNGFR SP: SEQ ID NO: 31) and transmembrane domains (CD8 TM: SEQ ID NO: 7; LNGFR TM: SEQ ID NO: 32; VSVG TM: SEQ ID 33) was determined by flow cytometry two days post transfection upon staining for a-CD3 scFV expression.

FIG. 9: Functional titer of CD8-targeted retroviral vector particles.

Retroviral vector particles were produced from transient transfected HEK293T cells. Functional titers were quantified by transducing SupT1 cells with serially diluted retroviral vector. Transduction efficiency was analyzed four days post transduction by quantification of marker positive cells. LV titers are represented as transducing units per volume (TU/mL), calculated by the ratio of transduced cells and applied LV volume. The titer of concentrated CD8-targeted CDV-pseudotyped retroviral vector particles displaying a modulating protein comprising an a-CD3 scFV (CD3mut scFV: SEQ ID NO: 4; CD3 scFV: SEQ ID NO: 5) expressed with different signal peptides (hCSF2R SP: SEQ ID NO: 15; Igκ SP: SEQ ID NO: 29; VSVG SP: SEQ ID NO: 30; CD8 SP: SEQ ID NO: 17; LNGFR SP: SEQ ID NO: 31) and transmembrane domains (CD8 TM: SEQ ID NO: 7; LNGFR TM: SEQ ID NO: 32; VSVG TM: SEQ ID 33) is shown. CD8-targeted CDV-pseudotyped retroviral particles (aCD8-CDV-LV) without display of an a-CD3 scFV was used as positive control.

FIG. 10: Incorporation of a-CD3 scFV within the LV envelope of CD8-targeted retroviral particles.

To confirm incorporation of a-CD3 scFV in functional retroviral particles GFP-encoding CD8-targeted CDV-pseudotyped retroviral particles (aCD8-CDV-LV) were immobilized via a N-terminal tag of the modulating protein on an ELISA plate. Unbound particles were removed by thorough washing with PBS. Subsequently SupT1 were added to the immobilized particles to allow transduction. The transduction efficiency was determined by quantification of GFP-expressing cells by flow cytometry eight days after transduction confirming presence of functional particles. CD8-targeted CDV-pseudotyped LV (aCD8-CDV-LV) without a-CD3 scFV display or wells without treatment of any LV (mock) were used as negative control. Incorporation of the modulating protein comprising an a-CD3 scFV (CD3mut scFV: SEQ ID NO: 4; CD3 scFV: SEQ ID NO: 5) expressed with different signal peptides (hCSF2R SP: SEQ ID NO: 15; Igκ SP: SEQ ID NO: 29; VSVG SP: SEQ ID NO: 30; CD8 SP: SEQ ID NO: 17; LNGFR SP: SEQ ID NO: 31) and transmembrane domains (CD8 TM: SEQ ID NO: 7; LNGFR TM: SEQ ID NO: 32; VSVG TM: SEQ ID 33) in functional retroviral vector particles are shown.

FIG. 11: Analysis of activation marker expression, proliferation and transduction efficiency upon transduction with CD8-targeted retroviral particles.

Non-activated cell-trace dye stained Pan T cells of three healthy donors were either left untreated (mock) or were transduced with GFP-encoding CD8-targeted CDV-pseudotyped retroviral vectors (aCD8-CDV-LV) with aCD8-CDV-LV alone (aCD8-CDV-LV), in presence of Transact™ (aCD8-CDV-LV TA) or with aCD8-CDV-LV containing a modulating protein comprising an a-CD3 scFV (CD3mut scFV: SEQ ID NO: 4; CD3 scFV: SEQ ID NO: 5) expressed with different signal peptides (hCSF2R SP: SEQ ID NO: 15; Igκ SP: SEQ ID NO: 29; VSVG SP: SEQ ID NO: 30; CD8 SP: SEQ ID NO: 17; LNGFR SP: SEQ ID NO: 31) and transmembrane domains (CD8 TM: SEQ ID NO: 7; LNGFR TM: SEQ ID NO 32, VSVG TM: SEQ ID 33). Transduction efficiency was analyzed by quantification of GFP-expressing cells using flow cytometry. Activation was quantified by staining for the activation marker CD25 and subsequent flow cytometry. T cells treated with Transact™ (mock TA) were used as positive control.
A CD25 expression of CD8+ and CD4+ T cells is shown two days after transduction.
B Transduction efficiency of CD8+ T cells eight days post transduction is shown.
C Transduction efficiency of CD4+ T cells eight days post transduction is shown.
D Exemplary data for the proliferation of T cells is shown eight days post transduction visualized by a fluorescent cell-trace dye.

FIG. 12: Incorporation of a-CD3 scFV within the envelope of measles virus, Nipah virus or VSV-G pseudotyped retroviral particles.

To confirm incorporation of a-CD3 scFV in functional retroviral particles GFP-encoding CD8-targeted Nipah-pseudotyped (aCD8-NiV-LV), biotin-specific measles pseudotyped (MV-LV) or VSV-G pseudotyped (VSV-G-LV) retroviral vectors were immobilized via a N-terminal tag of the modulating protein in an ELISA plate. Unbound particles were removed by thorough washing with PBS. Subsequently SupT1 (for VSV-G-LV and aCD8-NiV-LV) and biotinylated SupT1 (for MV-LV) were added to the immobilized particles to allow transduction. The transduction efficiency was determined by quantification of GFP-expressing cells by flow cytometry six days after transduction confirming presence of functional particles. Retroviral vector particles without a-CD3 scFV display (VSV-G-LV, aCD8-NiV-LV, MV-LV) or wells without treatment of any LV (mock) were used as negative control. Incorporation of a modulating protein comprising an a-CD3 scFV (SEQ ID NO: 4) expressed with a hCSF2R signal peptide (SP) (SEQ ID NO: 15) and CD8 transmembrane domains (TM) (SEQ ID NO: 7) with or without viral incorporation motif (gp41) (SEQ ID NO: 6) in functional retroviral vector particles are shown.
A Incorporation of the modulating protein within the envelope of VSV-G pseudotyped retroviral particles is shown.
B Incorporation of the modulating protein within the envelope of aCD8-Nipah virus pseudotyped retroviral particles is shown.
C Incorporation of the modulating protein within the envelope of biotin-specific measles virus pseudotyped retroviral particles is shown.

FIG. 13: Analysis of activation marker expression, proliferation and transduction efficiency upon transduction with retroviral particles of different pseudotypes.

Non-activated cell-trace dye stained Pan T cells of three healthy donors were either left untreated (mock) or were transduced with GFP-encoding VSV-G-, biotin-specific CDV- or MV-pseudotyped retroviral vectors containing a modulating protein comprising an a-CD3-scFV (SEQ ID NO: 4) expressed with a hCSF2R signal peptide (SP) (SEQ ID NO: 15) and CD8 transmembrane domains (TM) (SEQ ID NO: 7) with or without viral incorporation motif (gp41) (SEQ ID NO: 6) at a dose of 5 TU/cell. Retroviral vector particles without a-CD3 scFV display (VSV-G-LV, CDV-LV, MV-LV) were used as control. Transduction efficiency was analyzed by quantification of GFP-expressing cells using flow cytometry. Activation was quantified by staining for the activation marker CD25 and subsequent flow cytometry. T cells treated with Transact™ (mock TA) were used as positive control.
A CD25 expression of CD8+ and CD4+ T cells is shown five days after transduction.
B Transduction efficiency of CD8+ and CD4+ T cells eight days post transduction is shown.
C Exemplary data for the proliferation of T cells is shown eight days post transduction visualized by a fluorescent cell-trace dye. Retroviral vector particles without a-CD3 scFV display (LV w/o display) were used as control.

FIG. 14: Surface expression of the modulating protein with different scFV sequences.

HEK293T cells were either left untreated (mock) or were transiently transfected with the plasmids encoding the modulating protein. Surface expression of the modulating protein comprising different a-CD3 scFV (SEQ ID NO: 4, SEQ ID NO: 34 and SEQ ID NO: 3), a hCSF2R2 signal peptide (SP) (SEQ ID NO: 15) and a CD8 transmembrane domain (TM) (SEQ ID NO: 7) with the viral incorporation motif (gp41) (SEQ ID NO: 6) was determined by flow cytometry two days post transfection upon staining for a-CD3 scFV expression.

FIG. 15: Functional titer of adapter-based and CD8-targeted retroviral vector particles displaying a modulation protein with varying scFV sequences.

Retroviral vector particles displaying the modulating protein comprising different a-CD3 scFV (SEQ ID NO: 4, SEQ ID NO: 34 and SEQ ID NO: 3), a hCSF2R2 signal peptide (SP) (SEQ ID NO: 15) and a CD8 transmembrane domain (TM) (SEQ ID NO: 7) with the viral incorporation motif (gp41) (SEQ ID NO: 6) were produced from transient transfected HEK293T cells. Functional titers were quantified by transducing SupT1 (CD8-targeted) and biotinylated SupT1 (adapter-based) cells with serially diluted retroviral vector. Transduction efficiency was analyzed four days post transduction by quantification of marker positive cells. LV titers are represented as transducing units per volume (TU/mL), calculated by the ratio of transduced cells and applied LV volume. Adapter-based (CDV-LV) and CD8-targeted CDV-pseudotyped retroviral particles (aCD8-CDV-LV) without display of an a-CD3 scFV were used as positive control.
A The titer of concentrated adapter-based CDV-pseudotyped retroviral vector particles (CDV-LV) is shown.
B The titer of concentrated CD8-targeted CDV-pseudotyped retroviral vector particles is shown.

FIG. 16: Analysis of activation marker expression, proliferation and transduction efficiency upon transduction with retroviral particles displaying a modulation protein with varying scFV sequences.

Non-activated cell-trace dye stained CD8+ T cells of four healthy donors were either left untreated (mock) or were transduced with GFP-encoding CD8-targeted CDV-pseudotyped retroviral vectors (aCD8-CDV-LV) or biotin-specific CDV-pseudotyped retroviral vector (CDV-LV) in presence (+) or absence (−) of CD8-specific adapter molecules at a dose of 5 TU/cell. Transduction with retroviral vector particles without modulating protein and with modulating protein comprising different a-CD3 scFV (SEQ ID NO: 4, SEQ ID NO: 34 and SEQ ID NO: 3), a hCSF2R2 signal peptide (SP) (SEQ ID NO: 15) and a CD8 transmembrane domain (TM) (SEQ ID NO: 7) with the viral incorporation motif (gp41) (SEQ ID NO: 6) was compared. Transduction efficiency was analyzed by quantification of GFP-expressing cells using flow cytometry. Activation was quantified by staining for the activation marker CD25 and subsequent flow cytometry.
A CD25 expression of CD8+ T cells is shown two days after transduction with aCD8-CDV-LV alone (aCD8-CDV-LV) or with aCD8-CDV-LV containing modulating proteins.
B CD25 expression of CD8+ T cells is shown two days after transduction with CDV-LV alone (CDV-LV) or with CDV-LV containing modulating proteins.
C Transduction efficiency of CD8+ T cells and event count of CD8+ GFP+ T cells eight days post transduction with CD8-targeted retroviral vector particles is shown.
D Transduction efficiency of CD8+ T cells and event count of CD8+ GFP+ T cells eight days post transduction with adapter-based retroviral vector particles is shown.
E Mean fluorescent intensity (MFI) of CellTrace™ Violet (CTV) stained T cells is shown eight days post transduction with aCD8-CDV-LV or Adapter-LV.

FIG. 17: In vivo generation of CD20 CAR-T cells with adapter-based retroviral vector particles. NSG mice were transplanted with human PBMC and left untreated or were i.p. injected with CD8-specific adapter molecules the next day. Subsequently, biotin-specific CDV-LVs encoding a CD20CAR with or without modulating protein (hCSF2R signal peptide (SP) SEQ ID NO: 15, a-CD3 scFV SEQ ID NO: 4; CD8 transmembrane domain (TM) SEQ ID NO: 7; with the viral incorporation motif (gp41) (SEQ ID NO: 6) were i.v. injected. Blood samples were collected 5 and 12 days post transduction. On day 15 post transduction, the mice were sacrificed and blood samples, bone marrow and spleen samples were analyzed for transduced T cells. Transduction efficiency was analyzed by quantification of marker positive (LNGFR) cells using flow cytometry.
A Schematic description of the experimental layout for the in vivo generation of CAR T cells is shown.
B Cell count of CD3+ T cells in blood at day five, twelve and 15 post injection of retroviral vector particles.
C Cell count of CD4+ and CD8+ T cells isolated from blood, spleen and bone marrow on day 15 post LV injection.
D Transduction efficiency of CD8+ T cells and event count of CD8+ LNGFR+ T cells isolated from blood five, twelve and 15 days post transduction with adapter-based retroviral vector particles are shown.

FIG. 18: CD3-targeted CDV-LVs.

GFP-encoding CDV pseudotyped retroviral vector particles targeted against CD3 (SEQ ID NO: 4 and SEQ ID NO: 5) were produced from transient transfected HEK293T cells. Functional titers were quantified by transducing Jurkat cells with serially diluted retroviral vector. CD4− targeted CDV-LVs (aCD4-CDV-LV) were used as positive control. Transduction efficiency was analyzed four days post transduction by quantification of marker positive cells. Exemplary dot plots of two retroviral vector particle dilutions are shown.

### DETAILED DESCRIPTION OF THE INVENTION

In a first aspect the present invention provides a pseudotyped retroviral vector particle for activating and transducing T cells, wherein said retroviral vector particle comprises a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2, wherein said retroviral vector particle comprises at least one nucleic acid sequence encoding a transgene, and wherein said retroviral vector particle is a lentiviral or gammaretroviral vector particle. Said transgene may be a CAR or a TCR.

Said modulating protein may comprise a functional ectodomain comprising an antigen binding domain specific for the antigen CD3, wherein said antigen binding domain such as a scFV specific for the antigen CD3 comprises SEQ ID NO:1 (VH) and SEQ ID NO:2 (VL), wherein the order of sequence is from N-terminus to C-terminus SEQ ID NO:1 (VH)-SEQ ID NO:2 (VL).

Said modulating protein may comprise a functional ectodomain comprising an antigen binding domain specific for the antigen CD3, wherein said antigen binding domain is a scFV specific for the antigen CD3 and comprises SEQ ID NO:1 and SEQ ID NO:2.

Said modulating protein may comprise a functional ectodomain comprising an antigen binding domain specific for the antigen CD3, wherein said antigen binding domain is a scFV specific for the antigen CD3 and comprises SEQ ID NO:3 or SEQ ID NO:4.

Preferentially the nucleic acid molecule encoding a transgene, may be a nucleic acid molecule encoding a chimeric antigen receptor.

Said pseudotyped retroviral vector particle as disclosed herein, wherein said modulating protein comprises (from N-terminus to C-terminus):
I) said functional ectodomain
II) a transmembrane domain
III) a cytoplasmic domain.

Said pseudotyped retroviral vector particle as disclosed herein, wherein said modulating protein comprises (from N-terminus to C-terminus):
I) a signal peptide
II) a functional ectodomain comprising an antigen binding domain specific for the antigen CD3, wherein said antigen binding domain such as a scFV specific for the antigen CD3 comprises SEQ ID NO:1 and SEQ ID NO:2, preferentially in the order of SEQ ID NO:1-SEQ ID NO:2,
III) a transmembrane domain
IV) a cytoplasmic domain, and
optionally a spacer between said functional ectodomain and said transmembrane domain; wherein said retroviral vector particle comprises at least one nucleic acid encoding a transgene, and wherein said retroviral vector particle is a lentiviral or gammaretroviral vector particle.

Said pseudotyped retroviral vector particle as disclosed herein, wherein said cytoplasmic domain of said modulating protein comprises a viral incorporation motif.

Said pseudotyped retroviral vector particle as disclosed herein, wherein said viral incorporation motif of said modulating protein is the incorporation motif of gp41 (ENV of HIV), preferentially comprising SEQ ID NO:6.

Said pseudotyped retroviral vector particle as disclosed herein, wherein said transmembrane domain of said modulating protein may be e.g. the transmembrane domain of CD8, CD28 or PDGFR.

Said pseudotyped retroviral vector particle as disclosed herein, wherein said transmembrane domain of said modulating protein may be the transmembrane domain of CD8 comprising SEQ ID NO: 7.

Said pseudotyped retroviral vector particle as disclosed herein, wherein said transmembrane domain of said modulating protein may be the transmembrane domain of CD28 comprising SEQ ID NO:27.

Said pseudotyped retroviral vector particle as disclosed herein, wherein said transmembrane domain of said modulating protein may be the transmembrane domain of PDGFR comprising SEQ ID NO:28.

Said pseudotyped retroviral vector particle as disclosed herein, wherein said signal peptide of said modulating protein may be a hCSF2R signal peptide, preferentially comprising SEQ ID NO:15, may be a CD8 signal peptide, preferentially comprising SEQ ID NO:17, may be a IgK signal peptide, preferentially comprising SEQ ID NO:29, or may be a VSV-G signal peptide, preferentially comprising SEQ ID NO:30, or may be a LNGFR signal peptide, preferentially comprising SEQ ID NO:31.

Said pseudotyped retroviral vector particle as disclosed herein, wherein said signal peptide of said modulating protein may be a signal peptide of a cytokine, or of a cytokine receptor, or of a chemokine, or of a chemokine receptor, or of an antibody.

Said pseudotyped retroviral vector particle as disclosed herein, wherein said retroviral vector particle comprises
A)
a) an envelope protein with antigen-binding and fusion activity, and wherein said envelope protein is protein G of a virus of the Rhabdoviridae family such as VSV-G,
b) said modulating protein,
wherein said Rhabdoviridae virus is a virus of the vesiculovirus genus such as the Vesicular stomatitis virus (VSV), or
B)
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a T cell, and wherein said envelope protein is protein H of a virus of the Paramyxoviridae family,
b) an envelope protein with fusion activity of a virus of the Paramyxoviridae family,
c) said modulating protein
wherein said target antigen expressed on the surface of said T cell is not CD3, and
wherein said Paramyxoviridae virus is a virus of the morbillivirus genus, or
C)
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a T cell, and wherein said envelope protein is protein G of a virus of the Paramyxoviridae family,
b) an envelope protein with fusion activity of a virus of the Paramyxoviridae family,
c) said modulating protein,
wherein said target antigen expressed on the surface of said T cell is not CD3, and
wherein said Paramyxoviridae virus is a virus of the Henipavirus genus.

Said polypeptide that specifically may bind a target antigen expressed on the surface of a T cell may be a polypeptide comprising an antigen binding domain specific for a target antigen expressed on the surface of a T cell.

Said pseudotyped retroviral vector particle as disclosed herein, wherein in said retroviral vector particle of B) said protein H of a virus of the Paramyxoviridae family lacks at least one part of the cytoplasmic region of said protein H, and wherein said envelope protein with fusion activity lacks at least one part of the cytoplasmic region of said envelope protein; or
wherein in said retroviral vector particle of C) said protein G of a virus of the Paramyxoviridae family lacks at least one part of the cytoplasmic region of said protein G, and wherein said envelope protein with fusion activity lacks at least one part of the cytoplasmic region of said envelope protein.

Said pseudotyped retroviral vector particle as disclosed herein, wherein said polypeptide that specifically binds to said target antigen expressed on the surface of a T cell is specific for the antigen CD4, or wherein said polypeptide that specifically binds to said target antigen expressed on the surface of a T cell is specific for the antigen CD8.

Said pseudotyped retroviral vector particle as disclosed herein, wherein said polypeptide comprising an antigen binding domain specific for said target antigen expressed on the surface of a T cell is specific for the antigen CD4 and comprises SEQ ID NO:8 (VL) and SEQ ID NO:9 (VH), preferentially in the order of sequence from N to C-terminus VL-VH, or wherein said polypeptide comprising an antigen binding domain specific for a target antigen expressed on the surface of a T cell is specific for the antigen CD8 and comprises SEQ ID NO:10 (VL) and SEQ ID NO:11 (VH), preferentially in the order of sequence from N to C-terminus VL-VH.

Said pseudotyped retroviral vector particle as disclosed herein, wherein said polypeptide comprising an antigen binding domain specific for said target antigen expressed on the surface of a T cell is specific for the antigen CD4 and comprises SEQ ID NO:25 or wherein said polypeptide comprising an antigen binding domain specific for a target antigen expressed on the surface of a T cell is specific for the antigen CD8 and comprises SEQ ID NO:26.

Said pseudotyped retroviral vector particle as disclosed herein, wherein said virus of the morbillivirus genus is a measles virus or the Edmonston strain of measles virus, or is a canine distemper virus (CDV) or the 5804P strain or the Madrid/16 strain of the CDV.

Said pseudotyped retroviral vector particle as disclosed herein, wherein said virus of the henipavirus genus is a Nipah virus or a Hendravirus.

Said pseudotyped retroviral vector particle as disclosed herein, wherein said virus of the morbillivirus genus is a CDV, and wherein said protein H is a N-terminal truncated protein H, and wherein said truncated protein H is HcΔ21-Δ32, and wherein said truncated protein H comprises the amino acid substitutions of positions D526A, I527S, S528A, R529A, Y547A and T548A as compared to the unmodified protein H set forth in in SEQ ID NO:12, and wherein said protein with fusion activity (protein F) is a C-terminal truncated protein F (as compared to the unmodified protein F set forth in SEQ ID NO:13), wherein said truncated protein is FcΔ26-FcΔ30, preferentially FcΔ30, and wherein the N-terminal signal peptide sequence comprising SEQ ID NO:14 is present in said truncated protein F.

Said pseudotyped retroviral vector particle as disclosed herein, wherein said at least one nucleic acid sequence encoding a transgene is a chimeric antigen receptor (CAR) specific for an antigen expressed on the surface of a second target cell such as a cancer cell, or wherein said at least one nucleic acid sequence encoding a transgene is a T cell receptor (TCR) with specificity for an antigen of a second target cell such as a cancer cell.

Said pseudotyped retroviral vector particle as disclosed herein, wherein said pseudotyped retroviral vector particle comprises at least a second nucleic acid sequence encoding at least a second transgene such as a cytokine such as IL7, IL15, or IL21 or a cytokine receptor, such as CD127.

In a further aspect the present invention provides a pseudotyped retroviral vector particle as disclosed herein for use in immunotherapy.

In a further aspect the present invention provides a pseudotyped retroviral vector particle as disclosed herein for use in treatment of a disease in a subject in need thereof.

The disease may be a cancer, an autoimmune disease or an infectious disease.

The transgene may be a CAR specific for a tumor associated antigen (TAA) or a tumor specific antigen (TSA) expressed on the surface of a cancer cell.

In another aspect the present invention provides a composition comprising
i) a pseudotyped retroviral vector particle for activating and transducing T cells comprising
a) an envelope protein with antigen-binding activity, wherein said envelope protein is a recombinant protein that does not interact with at least one of its native receptors and is fused at its ectodomain to a polypeptide that specifically binds to a tag of a tagged polypeptide,
b) an envelope protein with fusion activity,
c) a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2, preferentially in the order from N-terminus to C-terminus of SEQ ID NO:1-SEQ ID NO:2,
ii) said tagged polypeptide, wherein said tagged polypeptide binds specifically to a target antigen expressed on the surface of a T cell, wherein said target antigen is not CD3, and wherein said retroviral vector particle comprises at least a nucleic acid sequence encoding a transgene, and wherein said retroviral vector particle is a lentiviral or gammaretroviral vector particle.

Said modulating protein may comprise a functional ectodomain comprising an antigen binding domain specific for the antigen CD3, wherein said antigen binding domain is a scFV specific for the antigen CD3 comprises SEQ ID NO:1 and SEQ ID NO:2, preferentially in the order from N-terminus to C-terminus of SEQ ID NO:1-SEQ ID NO:2.

Said modulating protein may comprise a functional ectodomain comprising an antigen binding domain specific for the antigen CD3, wherein said antigen binding domain is a scFV specific for the antigen CD3 comprises SEQ ID NO:3 or SEQ ID NO:4.

Said polypeptide that specifically binds to a tag of a tagged polypeptide may be a polypeptide comprising an antigen binding domain specific for a tag of a tagged polypeptide.

Said composition as disclosed herein, wherein said modulating protein comprises (from N-terminus to C-terminus):
I) said functional ectodomain
II) a transmembrane domain
III) a cytoplasmic domain.

Said composition as disclosed herein, wherein said modulating protein comprises (from N-terminus to C-terminus):
I) a signal peptide
II) a functional ectodomain comprising an antigen binding domain specific for the antigen CD3, wherein said antigen binding domain such as a scFV specific for the antigen CD3 comprises SEQ ID NO:1 and SEQ ID NO:2, preferentially in the order of SEQ ID NO:1-SEQ ID NO:2,
III) a transmembrane domain
IV) a cytoplasmic domain, and
optionally a spacer between said functional ectodomain and said transmembrane domain; wherein said retroviral vector particle comprises at least one nucleic acid encoding a transgene, and wherein said retroviral vector particle is a lentiviral or gammaretroviral vector particle.

Said composition as disclosed herein, wherein said cytoplasmic domain comprises a viral incorporation motif.

Said composition as disclosed herein, wherein said viral incorporation motif is the incorporation motif of gp41 (ENV of HIV), preferentially comprising SEQ ID NO:6.

Said composition as disclosed herein, wherein said transmembrane domain of said modulating protein may be e.g. the transmembrane domain of CD8, CD28 or PDGFR.

Said composition as disclosed herein, wherein said signal peptide of said modulating protein may be a hCSF2R signal peptide, preferentially comprising SEQ ID NO:15, may be a CD8 signal peptide, preferentially comprising SEQ ID NO:17, may be a Igκ signal peptide, preferentially comprising SEQ ID NO:29, or may be a VSV-G signal peptide, preferentially comprising SEQ ID NO:30, or may be a LNGFR signal peptide, preferentially comprising SEQ ID NO: 31.

Said pseudotyped retroviral vector particle as disclosed herein, wherein said signal peptide of said modulating protein may be a signal peptide of a cytokine, or of a cytokine receptor, or of a chemokine, or of a chemokine receptor, or of an antibody.

Said composition as disclosed herein, wherein
A)
i) said pseudotyped retroviral vector particle comprises:
a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and wherein said envelope protein is protein H of a virus of the Paramyxoviridae family,
b) an envelope protein with fusion activity of a virus of the Paramyxoviridae family,
c) a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2,
ii) said tagged polypeptide, wherein said tagged polypeptide binds specifically to a target antigen expressed on the surface of a T cell, wherein said target antigen is not CD3, and wherein said retroviral vector particle comprises at least a nucleic acid encoding a transgene, wherein said Paramyxoviridae virus is a virus of the morbillivirus genus, and wherein said retroviral vector particle is a lentiviral or gammaretroviral vector particle; or
B)
i) said pseudotyped retroviral vector particle comprises:
a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and wherein said envelope protein is protein G of a virus from the Paramyxoviridae family,
b) an envelope protein with fusion activity of a virus of the Paramyxoviridae family,
c) a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2,
ii) said tagged polypeptide, wherein said tagged polypeptide binds specifically to a target antigen expressed on the surface of a T cell, wherein said antigen is not CD3, and wherein said retroviral vector particle comprises at least a nucleic acid sequence encoding a transgene, wherein said Paramyxoviridae virus is a virus of the Henipavirus genus, and wherein said retroviral vector particle is a lentiviral or gammaretroviral vector particle.

Said composition as disclosed herein, wherein in said retroviral vector particle of A) said protein H of a virus of the Paramyxoviridae family lacks at least one part of the cytoplasmic region of said protein H, and wherein said envelope protein with fusion activity lacks at least one part of the cytoplasmic region of said envelope protein; or
wherein in said retroviral vector particle of B) said protein G of a virus of the Paramyxoviridae family lacks at least one part of the cytoplasmic region of said protein H, and wherein said envelope protein with fusion activity lacks at least one part of the cytoplasmic region of said envelope protein.

Said virus of the morbillivirus genus may be a measles virus or the Edmonston strain of measles virus, or may be a canine distemper virus (CDV) or the 5804P strain or the Madrid/16 strain of the CDV.

Said virus of the henpivirus genus may be a Nipah virus, or is a Hendravirus.

Said virus of the morbillivirus genus may be a CDV, and wherein said protein His a N-terminal truncated protein H, and wherein said truncated protein H is HcΔ21-Δ32, and wherein said truncated protein H comprises the amino acid substitutions of positions D526A, I527S, S528A, R529A, Y547A and T548A as compared to the unmodified protein H set forth in SEQ ID NO: 12, and wherein said protein with fusion activity (protein F) is a C-terminal truncated protein F (as compared to the unmodified protein F set forth in SEQ ID NO:13), wherein said truncated protein is FcΔ26-FcΔ30, preferentially FcΔ30, and wherein the N-terminal signal peptide sequence comprising SEQ ID NO:14 is present in said truncated protein F.

Said composition as disclosed herein, wherein said tagged polypeptide of A) or B) specific for said target antigen expressed on the surface of a T cell is specific for the antigen CD4 or for the antigen CD8.

Said composition as disclosed herein, wherein said tagged polypeptide of A) and/or B) specific for said target antigen expressed on the surface of a T cell is specific for the antigen CD4 and comprises SEQ ID NO:8 (VL) and SEQ ID NO:9 VH), or is specific for the antigen CD8 and comprises SEQ ID NO:10 (VL) and SEQ ID NO:11 (VH).

Said composition, wherein said at least one nucleic acid sequence encoding a transgene is a chimeric antigen receptor (CAR) specific for an antigen expressed on the surface of a second target cell such as a cancer cell, or wherein said at least one nucleic acid encoding a transgene is a T cell receptor (TCR) with specificity for an antigen of a second target cell such as a cancer cell.

In a further aspect the present invention provides a composition as disclosed herein for use in immunotherapy.

In a further aspect the present invention provides a composition as disclosed herein for use in treatment of a disease in a subject in need thereof.

The disease may be a cancer, an autoimmune disease or an infectious disease.

The transgene may be a CAR specific for a tumor associated antigen (TAA) or a tumor specific antigen (TSA) expressed on the surface of a cancer cell.

In a further aspect the present invention provides a combination (or a combinations of compositions) comprising
i) (a first composition comprising) a pseudotyped retroviral vector particle for activating and transducing T cells comprising
a) an envelope protein with antigen-binding activity, wherein said envelope protein is a recombinant protein that does not interact with at least one of its native receptors and is fused at its ectodomain to a polypeptide that specifically binds to a tag of a tagged polypeptide,
b) an envelope protein with fusion activity,
c) a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2, preferentially in the order from N-terminus to C-terminus of SEQ ID NO:1-SEQ ID NO:2,
ii) (a second composition comprising) said tagged polypeptide, wherein said tagged polypeptide binds specifically to a target antigen expressed on the surface of a T cell, wherein said target antigen is not CD3, and wherein said retroviral vector particle comprises at least a nucleic acid sequence encoding a transgene, and wherein said retroviral vector particle is a lentiviral or gammaretroviral vector particle.

In another aspect the present invention provides a pharmaceutical composition comprising
a) a pseudotyped retroviral vector particle for activating and transducing T cells, wherein said retroviral vector particle comprises a modulating protein comprising a functional ectodomain comprising an antigen binding domain specific for the antigen CD3, wherein said antigen binding domain such as a scFV specific for the antigen CD3 comprises SEQ ID NO: 1 and SEQ ID NO: 2, wherein said retroviral vector particle comprises at least one nucleic acid sequence encoding a transgene, and wherein said retroviral vector particle is a lentiviral or gammaretroviral vector particle; and optionally
b) a pharmaceutically acceptable carrier.

Pharmaceutically acceptable carriers, diluents or excipients may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art.

Said pseudotyped retroviral vector particle may comprise a polypeptide comprising an antigen binding domain specific for a target antigen expressed on the surface of a T cell as disclosed herein, and wherein said polypeptide is specific for the antigen CD4 or for the antigen CD8.

In another aspect the present invention provides a pharmaceutical composition comprising
i) a pseudotyped retroviral vector particle for activating and transducing T cells comprising
a) an envelope protein with antigen-binding activity, wherein said envelope protein is a recombinant protein that does not interact with at least one of its native receptors and is fused at its ectodomain to a polypeptide that specifically binds to a tag of a tagged polypeptide,
b) an envelope protein with fusion activity,
c) a modulating protein comprising a functional ectodomain comprising an antigen binding domain specific for the antigen CD3, wherein said antigen binding domain such as a scFV specific for the antigen CD3 comprises SEQ ID NO: 1 and SEQ ID NO:2,
ii) said tagged polypeptide, wherein said tagged polypeptide binds specifically to a target antigen expressed on the surface of a T cell, wherein said target antigen is not CD3, and wherein said retroviral vector particle comprises at least a nucleic acid encoding a transgene, and wherein said retroviral vector particle is a lentiviral or gammaretroviral vector particle; and optionally
c) a pharmaceutically acceptable carrier.

Said tagged polypeptide may comprise an antigen binding domain specific for a target antigen expressed on the surface of a T cell as disclosed herein, and wherein said polypeptide is specific for the antigen CD4 or for the antigen CD8.

In another aspect, the present invention provides an in vitro method for the generation of a sample of genetically modified T cells comprising
a) providing a sample comprising T cells
b) genetic modification of the T cells by targeted transduction with a pseudotyped retroviral vector particle, wherein said retroviral vector particle comprises a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2,
wherein said retroviral vector particle comprises at least one nucleic acid sequence encoding a transgene, and wherein said retroviral vector particle is a lentiviral or gammaretroviral vector particle; thereby generating a sample of genetically modified T cells.

Said in vitro method, wherein said retroviral vector particle comprises
A)
a) an envelope protein with antigen-binding and fusion activity, and wherein said envelope protein is protein G of a virus of the Rhabdoviridae family,
b) said modulating protein, wherein said Rhabdoviridae virus is a virus of the Vesiculovirus genus such as the Vesicular stomatitis virus (VSV), or
B)
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a T cell, and wherein said envelope protein is protein H of a virus of the Paramyxoviridae family,
b) an envelope protein with fusion activity of a virus of the Paramyxoviridae family,
c) said modulating protein,
wherein said target antigen expressed on the surface of said T cell is not CD3, and
wherein said Paramyxoviridae virus is a virus of the morbillivirus genus, or
C)
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a T cell, and wherein said envelope protein is protein G of a virus of the Paramyxoviridae family,
b) an envelope protein with fusion activity of a virus of the Paramyxoviridae family,
c) said modulating protein,
wherein said target antigen expressed on the surface of said T cell is not CD3, and
wherein said Paramyxoviridae virus is a virus of the Henipavirus genus.

Said in vitro method, wherein said retroviral vector particle comprises
a) an envelope protein with antigen-binding activity, wherein said envelope protein is a recombinant protein that does not interact with at least one of its native receptors and is fused at its ectodomain to a polypeptide that specifically binds to a tag of a tagged polypeptide,
b) an envelope protein with fusion activity,
c) a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2,
and wherein said pseudotyped retroviral vector is in combination with said tagged polypeptide, wherein said tagged polypeptide binds specifically to a target antigen expressed on the surface of a T cell, wherein said target antigen is not CD3.

Said in vitro method, wherein said sample comprising T cells is prepared by centrifugation before said step of modification.

Said in vitro method, wherein the number of T cells in said generated sample is less than 10-fold higher compared to the number of T cells in said sample comprising T cells.

Said in vitro method, wherein said method does not comprise an expansion step.

Said in vitro method, wherein said method is performed in less than 144 hours, less than 120 hours, less than 96 hours, less than 72 hours, less than 48 hours, less than 24 hours, less than 12 hours, less than 6 hours, less than 2 hours, less than hours, less than 30 minutes, or less than 15 minutes.

Said in vitro method, wherein said method is performed in a closed system. Said in vitro method, wherein said method is performed in a closed system, and wherein said method is an automated method.

In another aspect the present invention provides an in vivo method for treating a disease in a subject in need thereof comprising administering to said subject a pseudotyped retroviral vector particle, wherein said retroviral vector particle comprises a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2, wherein said retroviral vector particle comprises at least one nucleic acid encoding a transgene, and wherein said retroviral vector particle is a lentiviral or gammaretroviral vector particle.

Said pseudotyped retroviral vector particle may comprise a polypeptide that specifically binds to a target antigen expressed on the surface of a T cell as disclosed herein, and wherein said polypeptide is specific for the antigen CD4 or for the antigen CD8.

In another aspect the present invention provides an in vivo method for treating a disease in a subject in need thereof comprising
i) administering to said subject a pseudotyped retroviral vector particle comprising
a) an envelope protein with antigen-binding activity, wherein said envelope protein is a recombinant protein that does not interact with at least one of its native receptors and is fused at its ectodomain to a polypeptide that specifically binds to a tag of a tagged polypeptide,
b) an envelope protein with fusion activity,
c) a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO:2, and
ii) administering to said subject said tagged polypeptide, wherein said tagged polypeptide binds specifically to a target antigen expressed on the surface of a T cell, wherein said target antigen is not CD3, and
wherein said retroviral vector particle comprises at least a nucleic acid sequence encoding a transgene, and wherein said retroviral vector particle is a lentiviral or gammaretroviral vector particle.

Said tagged polypeptide that specifically binds to a target antigen expressed on the surface of a T cell as disclosed herein, wherein said polypeptide is specific for the antigen CD4 or for the antigen CD8.

The administration of the pseudotyped retroviral vector particle to said subject may be performed before, simultaneously or after the administration of the tagged polypeptide.

In another aspect the present invention provides a plasmid vector system (a kit) for generation of a pseudotyped retroviral vector particle for activating and transducing T cells comprising
a) a nucleic acid sequence encoding the envelope for a pseudotyped retroviral vector particle,
b) a nucleic acid sequence encoding a modulating protein comprising a functional ectodomain comprising an antigen binding domain specific for the antigen CD3, wherein said antigen binding domain such as a scFV specific for the antigen CD3 comprises SEQ ID NO: 1 and SEQ ID NO: 2,
c) a nucleic acid sequence encoding gag/pol of HIV-1,
d) a nucleic acid sequence encoding a transgene, wherein said retroviral vector particle is a lentiviral or gammaretroviral vector particle.

In another aspect the present invention provides a plasmid vector system (a kit) for generation of a pseudotyped retroviral vector particle for activating and transducing T cells comprising
a) a nucleic acid sequence encoding the envelope for a pseudotyped retroviral vector particle, wherein said retroviral vector particle comprises
i) an envelope protein with antigen-binding activity, wherein said envelope protein is a recombinant protein that does not interact with at least one of its native receptors and is fused at its ectodomain to a polypeptide that specifically binds to a tag of a tagged polypeptide,
ii) an envelope protein with fusion activity,
b) a nucleic acid sequence encoding a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO: 2,
c) a nucleic acid sequence encoding gag/pol of HIV-1
d) a nucleic acid sequence encoding a transgene,
wherein said pseudotyped retroviral vector, when expressed, is in combination with said tagged polypeptide, wherein said tagged polypeptide binds specifically to a target antigen expressed on the surface of a T cell, wherein said target antigen is not CD3,
wherein said retroviral vector particle is a lentiviral or gammaretroviral vector particle.

In another aspect the present invention provides a plasmid vector system (a kit) for generation of a pseudotyped retroviral vector particle for activating and transducing T cells comprising
a) a nucleic acid sequence encoding the envelope for a pseudotyped retroviral vector particle, wherein said retroviral vector particle comprises
i) an envelope protein with antigen-binding activity, wherein said envelope protein is a recombinant protein that does not interact with at least one of its native receptors and is fused at its ectodomain to a polypeptide that specifically binds to a target antigen expressed on the surface of a T cell, and wherein said envelope protein is protein H of a virus of the Paramyxoviridae family,
ii) an envelope protein with fusion activity of a virus of the Paramyxoviridae family,
b) a nucleic acid sequence encoding a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO: 2,
c) a nucleic acid sequence encoding gag/pol of HIV-1
d) a nucleic acid sequence encoding a transgene,
wherein said target antigen expressed on the surface of said T cell is not CD3, and
wherein said Paramyxoviridae virus is a virus of the morbillivirus genus, and
wherein said retroviral vector particle is a lentiviral or gammaretroviral vector particle.

In another aspect the present invention provides a plasmid vector system (a kit) for generation of a pseudotyped retroviral vector particle for activating and transducing T cells comprising
a) a nucleic acid sequence encoding the envelope for a pseudotyped retroviral vector particle, wherein said retroviral vector particle comprises
i) an envelope protein with antigen-binding activity, wherein said envelope protein is a recombinant protein that does not interact with at least one of its native receptors and is fused at its ectodomain to a polypeptide that specifically binds to a target antigen expressed on the surface of a T cell, and wherein said envelope protein is protein G of a virus of the Paramyxoviridae family,
ii) an envelope protein with fusion activity of a virus of the Paramyxoviridae family,
b) a nucleic acid sequence encoding a modulating protein comprising a functional ectodomain comprising an antigen binding domain specific for the antigen CD3, wherein said antigen binding domain such as a scFV specific for the antigen CD3 comprises SEQ ID NO:1 and SEQ ID NO: 2,
c) a nucleic acid sequence encoding gag/pol of HIV-1
d) a nucleic acid sequence encoding a transgene,
wherein said target antigen expressed on the surface of said T cell is not CD3, and
wherein said Paramyxoviridae virus is a virus of the Henipavirus genus, and
wherein said retroviral vector particle is a lentiviral or gammaretroviral vector particle.

Said nucleic acid sequence encoding a pseudotyped retroviral vector particle, wherein nucleic acid sequence encoding said modulating protein comprises:
I) a signal peptide
II) said functional ectodomain
III) a transmembrane domain
III) a cytoplasmic domain.

### EMBODIMENTS

In one embodiment of the invention the pseudotyped retroviral vector particle for activating and transducing T cells comprises a nucleic acid sequence encoding in 5′ to 3′ order a modulating protein comprising
I) a signal peptide
II) a functional ectodomain comprising an antigen binding domain specific for the antigen CD3, wherein said antigen binding domain such as a scFV specific for the antigen CD3 comprises SEQ ID NO: 1 and SEQ ID NO:2,
III) a transmembrane domain
IV) a cytoplasmic domain, and
optionally a spacer between said functional ectodomain and said transmembrane domain; wherein said retroviral vector particle comprises at least one nucleic acid encoding a transgene, and wherein said retroviral vector particle is a lentiviral or gammaretroviral vector particle.

In one embodiment of the invention the pseudotyped retroviral vector particle for activating and transducing T cells comprises a nucleic acid sequence encoding in 5′ to 3′ order a modulating protein comprising
I) a hCSF2R signal peptide, preferentially comprising SEQ ID NO:15
II) a functional ectodomain comprising an antigen binding domain specific for the antigen CD3, wherein said antigen binding domain such as a scFV specific for the antigen CD3 comprises SEQ ID NO:1 and SEQ ID NO:2,
III) a CD8 transmembrane domain, preferentially comprising SEQ ID NO:7
IV) a cytoplasmic domain comprising an incorporation motif, preferentially the incorporation motif of gp41 of HIV-1, preferentially comprising SEQ ID NO:6
optionally a spacer, preferentially a CD8 spacer, between said functional ectodomain and said transmembrane domain, preferentially comprising SEQ ID NO:16;
wherein said retroviral vector particle comprises at least one nucleic acid encoding a transgene, and wherein said retroviral vector particle is a lentiviral or gammaretroviral vector particle.

In one embodiment of the invention the pseudotyped retroviral vector particle for activating and transducing T cells comprises a nucleic acid sequence encoding in 5′ to 3′ order a modulating protein comprising
I) a hCD8 signal peptide, preferentially comprising SEQ ID NO:17
II) a functional ectodomain comprising an antigen binding domain specific for the antigen CD3, wherein said antigen binding domain such as a scFV specific for the antigen CD3 comprises SEQ ID NO:1 and SEQ ID NO:2,
III) a CD8 transmembrane domain, preferentially comprising SEQ ID NO:7
IV) a cytoplasmic domain comprising an incorporation motif, preferentially the incorporation motif of gp41 of HIV-1, preferentially comprising SEQ ID NO:6
optionally a spacer, preferentially a CD8 spacer, between said functional ectodomain and said transmembrane domain, preferentially comprising SEQ ID NO:16;
wherein said retroviral vector particle comprises at least one nucleic acid encoding a transgene, and wherein said retroviral vector particle is a lentiviral or gammaretroviral vector particle.

In one embodiment of the invention the pseudotyped retroviral vector particle for activating and transducing T cells comprises a nucleic acid sequence encoding in 5′ to 3′ order a modulating protein comprising
I) a signal peptide
II) a functional ectodomain comprising an antigen binding domain specific for the antigen CD3, wherein said antigen binding domain such as a scFV specific for the antigen CD3 comprises SEQ ID NO:1 and SEQ ID NO:2,
III) a transmembrane domain
IV) a cytoplasmic domain, comprising a co-stimulatory domain, preferentially the co-stimulatory domain of 4-1BB, preferentially comprising the SEQ ID NO:23, and
optionally a spacer between said functional ectodomain and said transmembrane domain; wherein said retroviral vector particle comprises at least one nucleic acid encoding a transgene, and wherein said retroviral vector particle is a lentiviral or gammaretroviral vector particle.

In one embodiment of the invention the pseudotyped retroviral vector particle for activating and transducing T cells comprises a nucleic acid sequence encoding in 5′ to 3′ order a modulating protein comprising
I) a signal peptide
II) a functional ectodomain comprising an antigen binding domain specific for the antigen CD3, wherein said antigen binding domain such as a scFV specific for the antigen CD3 comprises SEQ ID NO:1 and SEQ ID NO:2,
III) a transmembrane domain
IV) a cytoplasmic domain, comprising a co-stimulatory domain, preferentially the co-stimulatory domain of CD28, preferentially comprising the SEQ ID NO:24, and
optionally a spacer between said functional ectodomain and said transmembrane domain;
wherein said retroviral vector particle comprises at least one nucleic acid encoding a transgene, and wherein said retroviral vector particle is a lentiviral or gammaretroviral vector particle.

A pseudotyped retroviral vector particle as disclosed herein may be used to transduce cells in vivo at an any effective dosage. In some embodiments, the viral particle is administered to a subject in vivo by application to the tissue, the organ or to the blood circulation of a subject in need of therapy.

In case of use of an adapter-based retroviral vector system as disclosed herein, the adapter (tagged polypeptide) may be administered to a subject in vivo—in addition to the viral particle—by application to the tissue, the organ or to the blood circulation of a subject in need of therapy, said administration of the said adapter may be before, simultaneous to or after the administration of said viral particle.

In some embodiments, the pseudotyped retroviral vector particle as disclosed herein may be administered via a route of parenteral, intravenous, intramuscular, subcutanous, intratumoral, intraperitoneal, or intralymphatic administration. In some embodiments, the viral particle may be administered multiple times.

In one embodiment of the invention the pseudotyped retroviral vector particle as disclosed herein may be administered intratumorally to a subject and thereby activates and transduces the T cell portion of the tumor-infiltrating lymphocytes at the tumor site.

In one embodiment of the invention the pseudotyped retroviral vector particle as disclosed herein may be administered intravenously to a subject, and thereby activates and transduces the T cells in the circulatory blood system.

In one embodiment of the invention the pseudotyped retroviral vector particle as disclosed herein may be administered by intra lymphnode injection to a subject, and thereby activates and transduces the T cells in the lymph node.

In one embodiment of the invention the pseudotyped retroviral vector particle as disclosed herein may be administered by intra splenic injection to a subject, and thereby activates and transduces the T cells in the spleen.

The pseudotyped retroviral vector particle as disclosed herein may also be delivered to a subject according to viral titer (TU/mL). The amount of the pseudotyped retroviral vector particle as disclosed herein directly injected may be determined by total TU and can vary based on both the volume that could be feasibly injected to the site and the type of tissue to be injected. In some embodiments, the viral titer delivered is about 1×10⁵ to 1×10⁶, about 1×10⁵ to 1×10⁷, 1×10⁵ to 1×10⁷, about 1×10⁶ to 1×10⁹, about 1×10⁷ to 1×10¹⁰, about 1×10⁷ to 1×10¹¹, or about 1×10⁹ to 1×10¹¹ TU.

In one embodiment of the invention the pseudotyped retroviral vector particle as disclosed herein may activate and transduce T cells that are provided in a cell culture, and thereby may activate and transduce the T cells of the cell culture. The transduced T cells may be expanded to a therapeutically effective amount. The expanded T cells may be subsequently administered to a subject in need thereof.

In one embodiment of the invention the pseudotyped retroviral vector particle as disclosed herein may activate and transduce T cells that are provided in a closed system that may be an automated manufacturing system, and thereby may activate and transduce the T cells of the cell culture. The transduced T cells may be expanded to a therapeutically effective amount. The expanded T cells may be subsequently administered to a subject in need thereof.

In some embodiments, the pseudotyping glycoproteins of the pseudotyped retroviral vector particle as disclosed herein or functional fragments thereof may be a full-length polypeptide, a functional fragment, a homolog, or a functional variant of Human immunodeficiency virus (HIV) gpl60, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, anti-CD3 scFV, (CD3), Vesicular stomatitis virus VSV-G, or Chandipura virus and Piry virus CNV-G or PRV-G.

In some embodiments, the viral envelope glycoprotein of the pseudotyped retroviral vector particle as disclosed herein may be a G protein from the Cocal vesiculovirus species or a functional fragment thereof.

The formulations and compositions of the present invention may comprise a combination of any number of the pseudotyped retroviral vector particle as disclosed herein, and optionally one or more additional pharmaceutical agents (polypeptides, polynucleotides, compounds etc.) formulated in pharmaceutically acceptable compositions for administration to a cell, tissue, organ, or a subject, either alone, or in combination with one or more other modalities of therapy.

In some embodiments, the one or more additional pharmaceutical agent further increases transduction efficiency of vectors.

In one embodiment of the invention the pseudotyped retroviral vector particle for activating and transducing T cells comprises a second modulating protein comprising
I) a signal peptide
II) a functional ectodomain comprising an antigen binding domain specific for but not limited to CD28, CD137, OX40 and ICOS
III) a transmembrane domain
IV) a cytoplasmic domain, and
optionally a spacer between said functional ectodomain and said transmembrane domain;
wherein said retroviral vector particle comprises at least one nucleic acid encoding a transgene, and wherein said retroviral vector particle is a lentiviral or gammaretroviral vector particle.

In one embodiment of the invention the pseudotyped retroviral vector particle for activating and transducing T cells comprises a second modulating protein comprising
I) a signal peptide
II) a functional ectodomain comprising a cytokine for example IL7 or IL21
III) a transmembrane domain
IV) a cytoplasmic domain, and
optionally a spacer between said functional ectodomain and said transmembrane domain;
wherein said retroviral vector particle comprises at least one nucleic acid encoding a transgene, and wherein said retroviral vector particle is a lentiviral or gammaretroviral vector particle.

All definitions, characteristics and embodiments defined herein with regard to the first aspect of the invention as disclosed herein also apply mutatis mutandis in the context of the other aspects of the invention as disclosed herein.

### Definitions

Unless defined otherwise, technical and scientific terms used herein have the same meaning as commonly understood by one of ordinary skill in the art to which this invention belongs. As used herein the term “comprising” or “comprises” is used in reference to compositions, methods, and respective component(s) thereof, that are essential to the method or composition, yet open to the inclusion of unspecified elements, whether essential or not.

Rhabdoviridae is a family of negative-strand RNA viruses in the order Mononegavirales. The Rhabodivirdae family comprises to date 17 known taxonomic groups of which the Vesiculovirus is the best studies one. Indiana vesiculovirus (formerly Vesicular stomatitis Indiana virus (VSIV or VSV)) is the prototypic member of the genus Vesiculovirus. Its RNA genome encodes codes five major proteins: G protein (G), large protein (L), phosphoprotein (P), matrix protein (M) and nucleoprotein (N). The envelope glycoprotein (G) mediates viral attachment by binding to the LDL receptor (LDLR) and induces rapid endocytosis followed by fusion of the viral membrane with the endosomal membrane. Because of the broad tropism over a range of species and cell types, protein G of the Vesicular stomatitis virus (VSV) of the Rhabdoviridae family is often used in retroviral vectors for pseudotyping.

Cocal vesiculovirus, that also belongs to the genus of vesiculoviruses in the family of rhabdoviridae, is the cause of vesicular stomatitis in mammals. It is most closely related to the Vesicular stomatitis Indiana virus, but serologically different. While they also provide a broad tropism, lentiviral vectors pseudotyped with Cocal glycoproteins were shown to be resistant against inactivation by human serum.

Retroviridae is a virus family with a single-stranded, diploid, positive-sense RNA genome that is reverse-transcribed into a DNA intermediate that is then incorporated into the host cell genome. Retroviridae-derived viruses are enveloped particles with a diameter of 80-120 nm. (Retro-/lenti-/gammaretro-) viral vectors are replication-deficient viral particles that are derived from the corresponding virus family. They contain Gag and Pol proteins, a single-stranded RNA genome and are usually pseudotyped with heterologous envelope proteins derived from other viruses. The RNA genome of said viral vectors do not contain any viral gene to produce viral progeny, but psi elements and LTRs that are required for efficient packing and reverse transcription into DNA. The DNA intermediate may contain a gene of interest under the control of a suitable promoter, for example, the CMV promoter and the gene of interest is expressed upon integration of said DNA into the genome of the host cell. The process of entering the host cell, delivering the RNA genome, integration and expression of the gene of interest is called transduction. The minimal requirements of a gammaretrovirus or lentivirus based viral vector has been well-described in the art.

In addition, integrase-deficient retroviral vectors (ID-RVs) have been developed that cannot integrate the retroviral vector genome in the host cell genome. ID-RVs are derived from conventional retroviral vectors but contain no or a mutated form of the retroviral integrase. Upon entry into the host cell, the retroviral vector genome is reverse-transcribed in the cytoplasm, delivered into the nucleus, but not stably integrated into the host cell genome. ID-RVs are useful tools to express the gene of interest transiently. The definition of retroviral vectors and transduction also extents the integration-deficient retroviral vectors and its application.

Lentivirus is a genus of Retroviridae that cause chronic and deadly diseases characterized by long incubation periods, in the human and other mammalian species. The best-known lentivirus is the Human Immunodeficiency Virus (HIV), which can efficiently infect nondividing cells, so lentiviral derived retroviral vectors are one of the most efficient methods of gene delivery.

Gammaretroviridae is a genus of the Retroviridae family. Representative species are the murine leukemia virus (MLV) and the feline leukemia virus (FLV).

Paramyxoviridae is a family of viruses in the order of Mononegavirales. There are currently 49 species in this family, divided among 7 genera. Diseases associated with this virus family include measles, mumps, and respiratory tract infections. Members of this virus family are enveloped viruses with a non-segmented, negative-strand RNA genome of about 16 kb. Two membrane proteins with two distinct functions appear as spikes on the virion surface. The H/HN/G proteins mediate binding to the receptor at the cell surface.

Thus, the term “(virus) envelope protein(s) that have antigen binding activity” as used herein refers to protein(s) on the viral envelope that are responsible for binding to complementary receptors or antigens on the cell membrane of a target cell. For Paramyxoviridae H, HN or G proteins are virus envelope protein(s) that have antigen binding activity.

Upon binding the H/HN/G proteins change their conformation that induces a process called fusion helper function, leading to subsequent conformational changes within the F protein that is mediating the fusion of the viral and cellular membrane. The capsid and viral genome may now enter and infect or transduce the host cell.

The term “(virus) envelope proteins(s) that have fusion activity” as used herein refers to protein(s) that initiate fusion of viral and cellular membrane. For Paramyxoviridae F proteins refer to virus envelope protein(s) that have fusion activity.

The term “ectodomain” or “extracellular part/domain” as used herein refers to a domain of a membrane protein that extends into the extracellular space (the space outside a cell or virion).

The term “activation” as used herein refers to inducing physiological changes of a cell that increase target cell function, proliferation and/or differentiation.

The term “pseudotyping” or “pseudotyped” as used herein refers to a viral vector particle bearing envelope glycoproteins derived from other viruses having envelopes. The host range of the lentiviral vectors or viral vector particles of the present invention can thus be expanded or altered depending on the type of cell surface receptor used by the glycoprotein.

The term “modulating protein” of a retroviral vector particle as used herein refers to a protein that may modulate, e.g. activate a T cell. This modulation or specifically this activation may be due to the binding of an antigen binding domain of said modulating protein to stimulatory or co-stimulatory receptors of T cells, e.g. CD3 expressed on the surface of T cells.

To generate retroviral vectors the gag, pol and env proteins needed to assemble the vector particle are provided in trans by means of a packaging cell line, for example, HEK293T. This is usually accomplished by transfection of the packaging cell line with one or more plasmids containing the gag, pol and env genes. For the generation of pseudotyped vectors, the env gene, originally derived from the same retrovirus as the gag and pol genes and as the RNA molecule or expression vector, is exchanged for the envelope protein(s) of a different enveloped virus. As an example, the F and H or HN or G protein of Paramyxoviridae is used.

Thus, an exemplary pseudotyped vector particle based on the HIV-1 retrovirus comprises the (1) HIV-1 Gag and Pol proteins, (2) an RNA molecule derived from the HIV-1 genome that may be used to generate a retroviral vector particle based on the HIV-1 genome lacking the gag, env, pol, tat, vif, vpr, vpu and nef genes, but still comprising the LTRs, the psi element and a CMV promoter followed by the gene to be transduced, for example, a gene for the GFP protein, and (3) the F and H proteins of measles virus, for example, in a truncated form.

The terms “native receptor” or “originally receptor” as used herein may be used interchangeably and refer to the receptor or antigen expressed on the cell surface of a cell that is bound by the naturally occurring virus envelope protein with antigen (receptor) binding activity. The native measles virus receptors are SLAM, nectin-4 and CD46. The native canine distemper virus receptors are SLAM and nectin-4. Nipahvirus envelope proteins use ephrin-B2 and ephrin-B3 as receptors for entry.

The term “one envelope protein with antigen-binding activity that does not interact with at least one of its native receptor(s)” as used herein means that said protein has reduced or ablated interaction with at least one receptor of a cell that is normally targeted by the virus having said protein as described elsewhere herein. Reduced interaction means that said truncated and/or mutated protein interacts with said at least one native receptor at least 50% less efficient, at least 60% less efficient, at least 70% less efficient, at least 80% less efficient, at least 90% less efficient, at least 95% less efficient, at least 99% less efficient compared to the non-mutated protein. Preferentially said protein does not interact anymore with said at least one of its native receptors. The interaction may be the binding of these two molecules to each other. The less efficient interaction may be a reduced affinity of said protein to its native receptor. Said envelope protein with antigen-binding activity may have more than one native receptors, then the reduction or ablation of interaction of one of these native receptors of said protein results in a reduced tropism of the vector particle. The more interactions of said protein with its native receptors are inhibited by mutation the more effective is the reduction of tropism of the vector particle.

In some cases it may be sufficient to inhibit the interaction of some but not all native receptors to said protein as the remaining interactions are not of relevance in the intended application or use of the retroviral vector particle as disclosed herein, e.g. when a native receptor is not expressed on any cell (target cells and non-target cells) in the environment of target cells that are intended to be transduced.

If an envelope protein with antigen-binding activity has more than 2 native receptors, e.g. 3 native receptors, then preferentially said protein does not interact with the majority of its native receptors, e.g. 2 from 3.

More preferentially, the envelope protein with antigen-binding activity does not interact with all of its native receptors.

The term “tropism” as used herein refers to the host range or specificity of a virus or retroviral vector. As used herein, the envelope protein with antigen-binding activity that is fused at its ectodomain to a polypeptide comprising an antigen binding domain defines the host range of the retroviral vector. For the adaptable retroviral vector system, the tagged polypeptide specific for antigen expressed on target cells defines the host range of the retroviral vector.

The term “target cell” as used herein refers to a cell which expresses an antigen (a marker) on its cell surface that should be recognized (bound) by the pseudotyped retroviral vector particle as disclosed herein or the tagged polypeptide of the adaptable system as disclosed herein. The target cell may be T cell, a primary T cell or a cell line derived from a T cell. The target cell may be a mammalian cell such as a murine cell, preferentially the target cell is a human cell. For selective retroviral vector particle pseudotyped with measles virus envelope proteins, the truncated protein H fused to the polypeptide comprising an antigen binding domain specific for an antigen expressed on a target cell or for a tag of a tagged polypeptide as disclosed herein must have mutations that generally reduce or ablate productive interactions with its native receptors. Such mutations are well-known in the art. A mutation that ablates interaction of measles H protein with CD46 is e.g. the point mutation at position Y481, F431, V451, Y452, A527, P486, I487, A428, L464, G546, S548, F549 wherein these amino acids are replaced with another amino acid and this mutation prevents or assists in preventing interaction of the H protein with CD46. Alternatively, replacement of all five consecutive residues 473 to 477 in H protein with alanine may prevent interaction of H protein with CD46. Any of the above cited mutations maybe combined with each other

For example, the following introduction of mutations ablates productive interaction of the measles H protein with CD46 and SLAM, respectively: Y481A R533A. (Nakamura et al. (2004), Nakamura et al. (2005), Vongpunsawad et al. (2004), Masse et al. (2002), Masse et al. (2004), Patterson et al. (1999)). In another embodiment, the Hmut protein also includes the mutations S548L and F549S, which lead to a more complete ablation of residual infectivity via CD46. Also, the mutation of the residues V451 and Y529 ablates productive interaction with CD46 and SLAM. Alternative mutations for ablating/preventing interaction of the H protein with CD46 have been described above. All of these mutations, which are introduced into the truncated H proteins in order to reduce or ablate the natural receptor usage, are located in the ectodomain of the measles H protein. For preventing interaction of the H protein with SLAM one of the following residues may be replaced with any other amino acid, in particular, alanine: I194, D530, Y553, T531, P554, F552, D505, D507.

For nectin-4, mutations within the H protein have been proposed in the art which abolish binding to this receptor as well. For example, Tahara et al. show that amino acid substitutions F483A, Y541S and Y543S of wt measles virus H protein result in an ablated fusion activity on Nectin-4 positive cells (Tahara et al. (2008)). This has been confirmed by Liu et al. showing that amino acid substitutions F543A and P497S of the Edmonston strain H abolish infection by vesicular stomatitis virus pseudotyped with Edmonston strain F and H envelope proteins (Liu et al. (2014)). There are further residues on the surface of the H molecule which are well conserved among different Morbilliviruses that may be involved in Nectin-4 dependent fusion, e.g. Phe483, Asp521, Leu522, Tyr524, Tyr541, Tyr543, Ser544, Arg547, Ser550, and Tyr551 (Tahara et al. (2008)). This suggests that further mutations might be helpful for preventing interaction with Nectin-4. Lentiviral or gammaretroviral vector particles pseudotyped with truncated F proteins and mutated H proteins additionally displaying at their ectodomain a polypeptide comprising an antigen binding domain specific for an antigen expressed on a target cell or for a tag of a tagged polypeptide, wherein said tagged polypeptide binds specifically to an antigen expressed on a target cell, no longer enter cells via CD46, SLAM and/or nectin-4, but are rather targeted to and enter only those cells displaying the respective corresponding markers at their surface.

A mutation that ablates interaction of canine distemper virus H protein with SLAM and Nectin 4 may be e.g. the point mutation at position D526, I527, S528, R529; Y547 and T548 according to amino acid number of SEQ ID NO:12, wherein amino these amino acids are replaced with another amino acid and this mutation prevents or assists in preventing interaction of the H protein with SLAM and Nectin-4 (Bah et al (2020); von Messing et al (2005)).

For selective retroviral vector particles pseudotyped with Nipahvirus envelope proteins reduced or ablated interactions of the G protein to the native receptors ephrin-B2 and ephrin-B3 is required. Residues within the G protein were identified by screening mutants resulting in variants with ablated receptor binding ability (Bender et al. (2016)). E501, W504, Q530, E533 were either single mutated or in combination. The combined mutation of E501A, W504A, Q530A, E533A showed completely ablated receptor binding ability for both receptors ephrin-B2 and ephrin-B3.

A pseudotyped retroviral vector particle “derived from”, for example, HIV-1, as used in the present invention, refers to a particle in which the genetic information for the RNA and/or the Gag and Pol proteins comprised by the vector particle originate from said retrovirus, in the above case, HIV-1. The original retroviral genome can comprise mutations, such as deletions, frame shift mutations and insertions.

The terms “cytoplasmic domain”, “cytoplasmic portion”, “cytoplasmic tail”, “cytoplasmic region”, “intracellular domain” or “endodomain”, as used in herein refer to the portion of the respective protein that is adjacent to the transmembrane domain of the protein and, if the protein is inserted into the membrane under physiological conditions, extends into the cytoplasm or in case of viral particles reaching into the intravirion side. Within Paramyxoviridae all envelope proteins with antigen-binding function are characterized to date as type II membrane proteins, meaning that the cytoplasmic domain is located at the N-terminus of the envelope protein.

In case of the cytoplasmic domain of the modulating protein, the cytoplasmic domain refers to the portion of the protein that reaches into the intravirion side. The portion of the protein that reaches into the intravirion side may comprise at least 1, at least 2, at least 3 or at least 4 amino acids. The cytoplasmic domain of the modulating protein may comprise a viral incorporation motif. Said incorporation motif may be the incorporation motif of gp41.

The term “truncated”, as used in the present invention, refers to a deletion of amino acid residues of the designated protein. It is clear to the skilled person that a protein is encoded by a nucleic acid. Thus, “truncated” also refers to the corresponding coding nucleic acids in a nucleic acid molecule that codes for a given “truncated” protein.

In the present invention, specific reference is made to “truncated H”, “truncated G” or “truncated F” proteins, which designates the Paramyxoviridae, preferably measles H protein, Nipah G protein and Nipah or measles F proteins, respectively, whose cytoplasmic portion has been partly or completely truncated, i.e. amino acid residues (or coding nucleic acids of the corresponding nucleic acid molecule encoding the protein) have been deleted.

The cytoplasmic portion of the F protein (termed Fc) is located at the C-terminus of the protein.

For all envelope proteins with the cytoplasmic portion located at the C-terminus one begins counting from the C-terminal end of the protein when ascertaining the desired sequence. As an example for measles F glycoprotein, FcΔ30 would refer to an F protein having deleted the last 30 amino acids counting from the C-terminal end of the protein F set forth in SEQ ID NO:18. Consequently, FcΔ30 would refer to an F protein having a cytoplasmic domain with the amino acid sequence SEQ ID NO:19. As an example for Canine distemper virus strain 5840P F glycoprotein, FcΔ30 would refer to an F protein having deleted the last 30 amino acids counting from the C-terminal end of the protein F set forth in SEQ ID NO: 13. Consequently, for the F protein derived from Canine distemper virus strain 5840P FcΔ30 would refer to an F protein having a cytoplasmic domain comprising amino acid sequence of SEQ ID NO:20.

By contrast, the cytoplasmic portion of the H, HN or G protein is located at the N-terminus (termed Hc).

Thus, one begins counting at the second amino acid residue of the N-terminal end of the H, HN or G protein (i.e. omitting the first methionine residue) when ascertaining the desired sequence. The term “truncated protein His HcΔ21-Δ32” in the context of the protein H of a CDV as used herein refers to any truncated protein H of the CDV having deleted the first 21 to 32 amino acids counting from the N-terminal end of the protein H set forth in SEQ ID NO: 12 (the unmodified protein H): individually said truncated protein H may be:

HcΔ21, HcΔ22, HcΔ23, HcΔ24, HcΔ25, HcΔ26, HcΔ27, HcΔ28, HcΔ29, HcΔ30, HcΔ31 or HcΔ32.

As an example, for the H protein derived from Canine Distemper virus strain 5840P the cytoplasmic domain of HcΔ21 comprises the amino acids as in SEQ ID NO:21. Accordingly, the H protein derived from Canine Distemper virus strain 5840P the cytoplasmic domain of HcΔ30 comprises the amino acids as in SEQ ID NO:22.

It is disclosed in WO2008037458A2 that the cytoplasmic domain of the measles F protein can be truncated to comprise at least 1 positively charged amino acid residue and the cytoplasmic portion of the H protein can be truncated to comprise at least 9 consecutive amino acid residues of the C-terminal cytoplasmic portion of the H protein plus an additional methionine at the N-terminus. However, a further truncation of the cytoplasmic portion of the H protein is expected to be feasible, if the H protein is truncated to allow efficient pseudotyping and still has fusion support function.

Modifications that allow truncation for efficient pseudotyping may be combined with modifications that ablate native receptor binding function.

The person skilled in the art will readily be able to introduce mutations as, for example, additions and deletions, into a given nucleic acid or amino acid sequence.

The proteins of the present invention further include functional homologs. A protein is considered a functional homolog of another protein for a particular function, if the homolog has a similar function as the original protein. The homolog can be, for example, a fragment of the protein, or a substitution, addition, or deletion mutant of the protein.

Determining whether two amino acid sequences are substantially homologous is typically based on FASTA searches. For example, the amino acid sequence of a first protein is considered to be homologous to that of a second protein if the amino acid sequence of the first protein shares at least about 70% amino acid sequence identity, preferably at least about 80% identity, and more preferably at least about 85%, 90%, 95% or 99% identity, with the sequence of the second protein.

The terms “Psi positive” and “psi negative”, as used in the present application, refer to a nucleic acid molecule where the retroviral psi element is present and absent, respectively. The psi element is a cis-acting signal located near the 5′ end of the retroviral genome and designates a packaging signal, which is of importance during assembly of the viruses and leads to the incorporation of the viral RNA into the viral core. Thus, a psi negative RNA does not comprise the retroviral psi element and consequently will not be assembled into a vector particle of the present invention; in contrast, a psi positive RNA that does comprise said psi element will be effectively assembled into the vector particle.

The term “antigen expressed on the surface of a (target) cell” or “cell (surface) marker”, as used in the present invention, refers to a molecule present on the surface of a cell, preferentially on a target cell. Such molecules can be, inter alia, peptides or proteins that may comprise sugar chains or lipids, clusters of differentiation (CDs), antibodies or receptors. Since not all populations of cells express the same cell markers, a cell marker can thus be used to identify, select or isolate a given population of cells expressing a specific cell marker. As an example, CD4 is a cell marker expressed by T helper cells, regulatory T cells, and monocytes. Thus, T helper cells, regulatory T cells, and monocytes can be identified, selected or otherwise isolated, inter alia by a FACS cell sorter, by means of the CD4 cell marker.

The term “tagged polypeptide” that is specific for binding to a target antigen expressed on the surface of a T cell as used herein refers to a polypeptide that has bound thereto directly or indirectly at least one additional component, i.e. the tag. The tagged polypeptide as used herein is able to bind a target antigen expressed on T cells, regularly CD4 or CD8. The polypeptide may be an antibody or antigen binding fragment thereof that binds to said antigen expressed on the surface of a target cell. The polypeptide of the tagged polypeptide alternatively may be a cytokine or a growth factor or another soluble polypeptide that is capable of binding to an antigen of a target cell.

The term “adapter” or “adapter molecule” as used herein refers to a tagged polypeptide that can bind to a target antigen of a T cell, e.g. antibody or antigen binding fragment thereof such as a scFV, and has bound thereto directly or indirectly at least one additional component, i.e. the tag. The adapter or adapter molecule may by a tagged antibody or antigen binding fragment thereof, a cytokine or a growth factor or another soluble polypeptide that is capable of binding to a target antigen of a T cell. The retroviral vector particle specific for a tag as disclosed herein may bind to said adapter.

Such an adaptable retroviral vector system, that comprises a pseudotyped retroviral vector particle specific for a tag and said tagged polypeptide specific for an antigen expressed on the surface of a target cell is disclosed e.g. in WO2019086351A1.

The tag of said tagged polypeptide may be e.g. a hapten or dextran and the hapten or dextran may be bound by the antigen binding domain of the polypeptide comprising an antigen binding domain specific for the tag. Said tag also may be a peptide such as a neopeptide.

Haptens such as e.g. FITC, biotin, PE, streptavidin, thiamin or dextran are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

The term “polypeptide comprising an antigen binding domain specific for a tag” as used herein refers to a polypeptide that can bind a tag of a tagged polypeptide. The tagged polypeptide is different from the polypeptide that comprises the antigen binding domain specific for the tag. The polypeptide comprising the antigen binding domain specific for a tag may be an antibody or antigen binding fragment thereof that binds to said tag of the tagged polypeptide.

The term “polypeptide comprising an antigen binding domain specific for a tag” as used herein refers to a polypeptide that can bind a tag of a tagged polypeptide. The polypeptide comprising the antigen binding domain specific for a tag may be an antibody or antigen binding fragment thereof such as a scFV or a nanobody that binds to said tag of the tagged polypeptide.

The term “antibody” as used herein is used in the broadest sense to cover the various forms of antibody structures including but not being limited to monoclonal and polyclonal antibodies (including full length antibodies), multispecific antibodies (e.g. bispecific antibodies), antibody fragments, i.e. antigen binding fragments of an antibody, immunoadhesins and antibody-immunoadhesin chimeras, that specifically recognize (i.e. bind) an antigen. “Antigen binding fragments” comprise a portion of a full-length antibody, preferably the variable domain thereof, or at least the antigen binding site thereof (“an antigen binding fragment of an antibody”). Examples of antigen binding fragments include Fab (fragment antigen binding), scFV (single chain fragment variable), single domain antibodies, diabodies, dsFv, Fab′, single-chain antibody molecules, and multispecific antibodies formed from antibody fragments.

As used herein, the term “antigen” is intended to include substances that bind to or evoke the production of one or more antibodies and may comprise, but is not limited to, proteins, peptides, polypeptides, oligopeptides, lipids, carbohydrates such as dextran, and combinations thereof, for example a glycosylated protein or a glycolipid. The term “antigen” as used herein refers to a molecular entity that may be expressed on the surface of a target cell and that can be recognized by means of the adaptive immune system including but not restricted to antibodies or TCRs, or engineered molecules including but not restricted to endogenous or transgenic TCRs, CARs, scFVs or multimers thereof, Fab-fragments or multimers thereof, antibodies or multimers thereof, single chain antibodies or multimers thereof, or any other molecule that can execute binding to a structure with high affinity.

The term “expression” as used herein is defined as the transcription and/or translation of a particular nucleotide sequence driven by its promoter in a cell.

As used herein, the term “subject” refers to an animal. Preferentially, the subject is a mammal such as mouse, rat, cow, pig, goat, chicken dog, monkey or human. More preferentially, the individual is a human. The subject may be a subject suffering from a disease such as cancer.

The term “administering” refers to local and systemic administration, e.g., including enteral, parenteral, pulmonary, and topical/transdermal administration. The administration may be directly intratumoral. Routes of administration for pharmaceutical ingredients include, e.g., oral administration, nasal or inhalation administration, administration as a suppository, topical contact, transdermal delivery, intrathecal administration, intravenous administration, intraperitoneal administration, intramuscular administration, intralesional administration, or subcutaneous administration to a subject. Administration can be by any route including parenteral and transmucosal (e.g., oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial.

A recombinant protein is a biotechnologically generated protein that does not occur naturally in a eukaryotic and/or prokaryotic cell. Often it is composed of different domains from different proteins, e.g. as used herein, a viral envelope protein is fused (at its ectodomain) to a polypeptide that comprises an antigen binding domain specific for an antigen or for a tag.

The terms “having specificity for”, “specifically binds” or “specific for” with respect to an antigen-binding domain of an antibody or a fragment thereof refer to an antigen-binding domain which recognizes and binds to a specific antigen, but does not substantially recognize or bind other molecules in a sample. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based cytotoxic responses to attack cancer cells. T cells that have a natural or genetically engineered reactivity to a patient's cancer are generated in vitro and then transferred back into the cancer patient or are directly generated in vivo. Then the immunotherapy is referred to as “CAR T cell immunotherapy”.

The term “treatment” as used herein means to reduce the frequency or severity of at least one sign or symptom of a disease.

The terms “therapeutically effective amount” or “therapeutically effective population” mean an amount of a cell population which provides a therapeutic benefit in a subject.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refer to the fact that cells, preferentially T cells can be manipulated by recombinant methods well known in the art to express stably or transiently peptides or proteins which are not expressed in these cells in the natural state. For example, T cells, preferentially human T cells are engineered to express an artificial construct such as a chimeric antigen receptor on their cell surface.

The terms “automated method” or “automated process” as used herein refer to any process being automated through the use of devices and/or computers and computer software. Methods (processes) that have been automated require less human intervention and less human time. In some instances the method of the present invention is automated if at least one step of the present method is performed without any human support or intervention. Preferentially the method of the present invention is automated if all steps of the method as disclosed herein are performed without human support or intervention other than connecting fresh reagents to the system. Preferentially the automated process is implemented on a closed system such as CliniMACS Prodigy® (Miltenyi Biotec).

The closed system may comprise a) a sample processing unit comprising an input port and an output port coupled to a rotating container (or centrifugation chamber) having at least one sample chamber, wherein the sample processing unit is configured to provide a first processing step to a sample or to rotate the container so as to apply a centrifugal force to a sample deposited in the chamber and separate at least a first component and a second component of the deposited sample; and b) a sample separation unit coupled to the output port of the sample processing unit, the sample separation unit comprising a separation column holder, a pump, and a plurality of valves configured to at least partially control fluid flow through a fluid circuitry and a separation column positioned in the holder, wherein the separation column is configured to separate labeled and unlabeled components of sample flown through the column.

Said rotating container may also be used as a temperature-controlled cell incubation and cultivation chamber (CentriCult Unit=CCU). This chamber may be flooded with defined gas mixes, provided by an attached gas mix unit (e.g. use of pressurized air/N2/CO2 or N2/CO2/O2).

All agents may be connected to the closed system before process initiation. This comprises all buffers, solutions, cultivation media and supplements, MicroBeads, used for washing, transferring, suspending, cultivating, harvesting cells or immunomagnetic cell sorting within the closed system. Alternatively, such agents might by welded or connected by sterile means at any time during the process.

The cell sample comprising T cells may be provided in transfer bags or other suited containers which can be connected to the closed system by sterile means.

The term “providing a (cell) sample comprising T cells” means the provision of a cell sample, preferentially of a human cell sample of hematologic origin. Normally, the cell sample may be composed of hematologic cells from a donor or a patient. Such blood product can be in the form of whole blood, buffy coat, leukapheresis, PBMCs or any clinical sampling of blood product. It may be from fresh or frozen origin.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

In general, T cells may be characterized based on their function and marker expression. Two main subgroups have been defined: CD4 expressing T cells (i.e. T helper cells) and CD8 expressing T cells (i.e. cytotoxic T cells). CD8 positive specifically lyse e.g. virus infected or tumor cells by releasing perforin, granzyme and FasL upon specific binding to the respective peptide presented on the MHC I to the TCR. On CD4+ T cells peptides presented on MHC II are bound specifically by the respective TCR inducing a signaling cascade triggering the release of several cytokines such as interferons and interleukins. Such cytokines may recruit other immune cells and may activate CD8+ T cells for a boosted and sustained cytolytic activity.

T cells differentiate into different phenotypes showing a specific memory or effector function profile.

Naive T cells (T_{N}) have recently undergone positive and negative selection in the thymus and are considered to be early differentiated with high memory function but a low effector function. They can be identified by flow cytometry expressing CD45RA, CCR7 and CD62L and being negative for CD45RO, CD95 and IL-2Rbeta. Naïve T cells in the blood are normally found in a quiescent state, which is characterized by small cell size, low proliferative capacity, low basal metabolic programs and low responsiveness to key cytokines, e.g. IL-2. The terms resting T cells”, “quiescent T cells”, “unstimulated T cells” and “non-activated T cells” may be used interchangeably.

Stem cell memory T cells (T_{SCM}) have a high potential for self-renewal, are minimally differentiated and can differentiate into other phenotypes. They can be identified by flow cytometry expressing CD45RA, CD45RO, CCR7, CD62L, CD95 and IL-2Rbeta.

Central memory T cells (T_{CM}) are characterized by a low effector function profile and a long persistence. Upon antigen encounter, this T cell subset expands rapidly and differentiate into T cells with effector function. They can be identified by flow cytometry expressing CD45RO, CCR7, CD62L, CD95 and IL-2Rbeta.

Effector memory T cells (T_{EM}) migrate to inflamed tissues and have an intermediate level of effector function. They can be identified by flow cytometry expressing CD45RO, CD95, IL-2Rbeta and being negative for CCR7 and CD62L.

Effector T cells (T_{EFF}) are short lived T cells with no memory function but the highest potential of cytolytic effector function. They can be identified by flow cytometry expressing CD45RA, CD95, IL-2Rbeta and being negative for CD45RO, CCR7 and CD62L.

In general, a chimeric antigen receptor (CAR) may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments the CAR may be an adaptable CAR system (similar to the adaptable retroviral vector system) and may be then referred to as “anti-tag” CAR or “adapterCAR” or “universal CAR” as disclosed e.g. in U.S. Pat. No. 9,233,125B2.

A “signal peptide” refers to a peptide sequence that directs the transport and localization of the protein within a cell during or post translation, e.g. to a certain cell organelle (such as the endoplasmic reticulum) and/or the cell surface.

Generally, an “antigen binding domain” of a CAR refers to the region of the CAR that specifically binds to an antigen, e.g. to a tumor associated antigen (TAA) or tumor specific antigen (TSA). The CARs of the invention may comprise one or more antigen binding domains (e.g. a tandem CAR). Generally, the targeting regions on the CAR are extracellular. The antigen binding domain of the CAR may comprise an antibody or an antigen binding fragment thereof. The antigen binding domain of the CAR may comprise, for example, full length heavy chain, Fab fragments, single chain Fv (scFV) fragments, nanobodies, divalent single chain antibodies or diabodies. Any molecule that binds specifically to a given antigen such as affibodies or ligand binding domains from naturally occurring receptors may be used as an antigen binding domain. Often the antigen binding domain of a CAR is a scFV. Normally, in a scFV the variable regions of an immunoglobulin heavy chain and light chain are fused by a flexible linker to form a scFV. Such a linker may be for example the “(G4S) 3-linker”.

In some instances, it is beneficial for the antigen binding domain of the CAR to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain of the CAR and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. Splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ.

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

If the CAR is an inhibitory CAR (referred to normally as “iCAR”), then said CAR may have the same extracellular and/or transmembrane domains as the activating CAR but differs from the activating CAR with regard to the endodmain.

The at least one endodomain of the inhibitory CAR may be a cytoplasmic signaling domain comprising at least one signal transduction element that inhibits an immune cell or comprising at least one element that induces apoptosis.

The CARs that may be transduced by the pseudotyped retroviral vector particle as disclosed herein present may be designed to comprise any portion or part of the above-mentioned domains as described herein in any order and/or combination resulting in a functional CAR.

The term “display” as used herein refers to a protein or peptide that is incorporated into the viral envelope, thereby presenting the extracellular domain outside the viral particle.

### EXAMPLES

The following examples are intended for a more detailed explanation of the invention but without restricting the invention to these examples.

### Example 1: Principle of the Anti-CD3-Displaying Retroviral Vector System

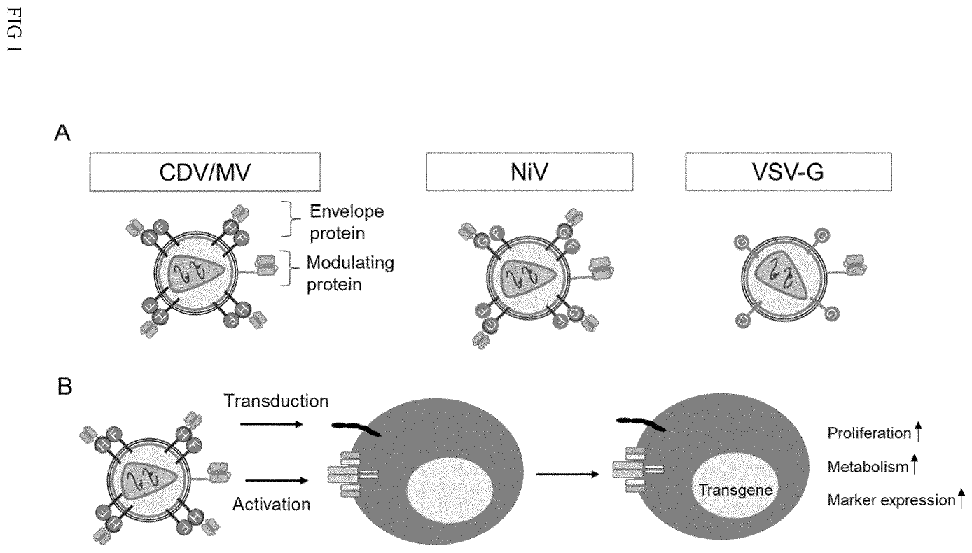

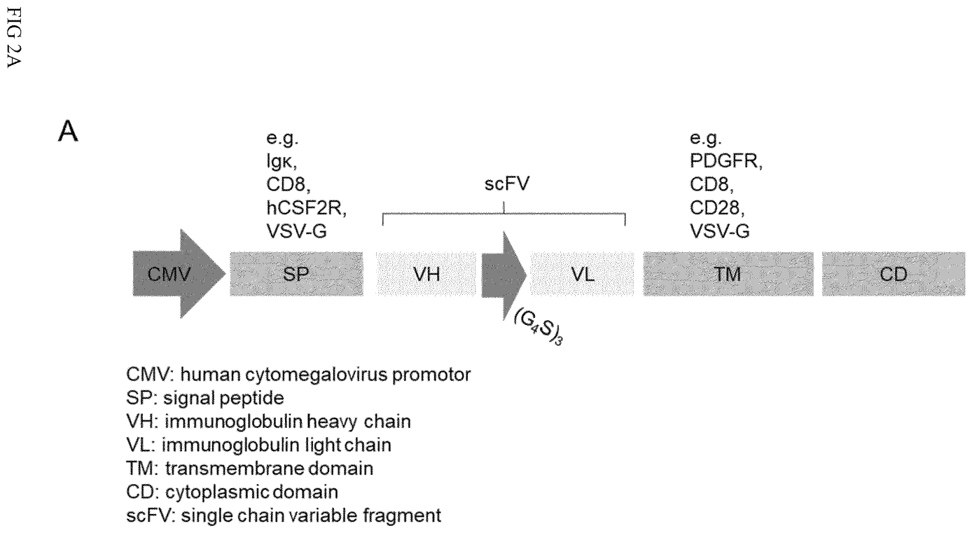

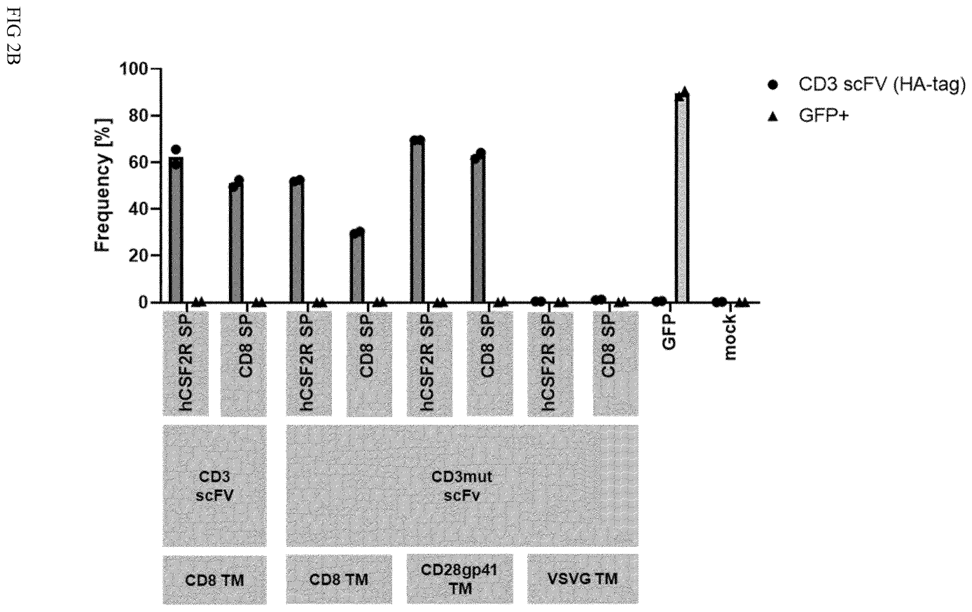

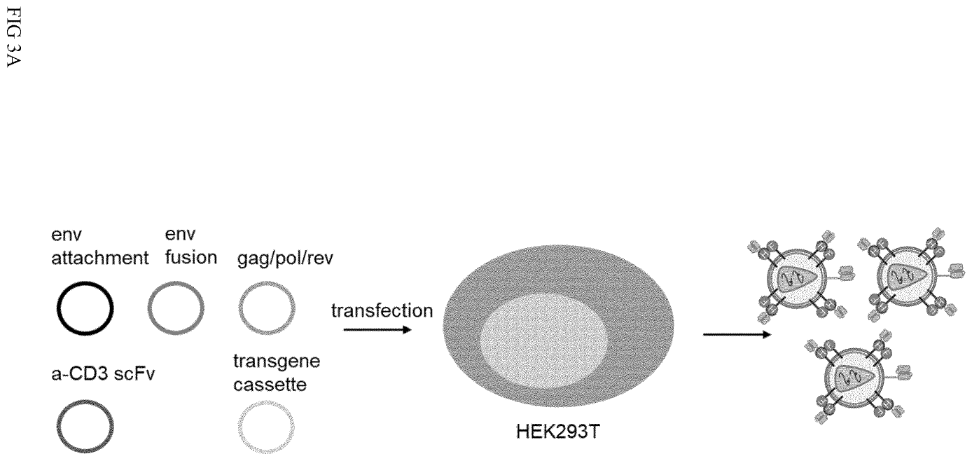

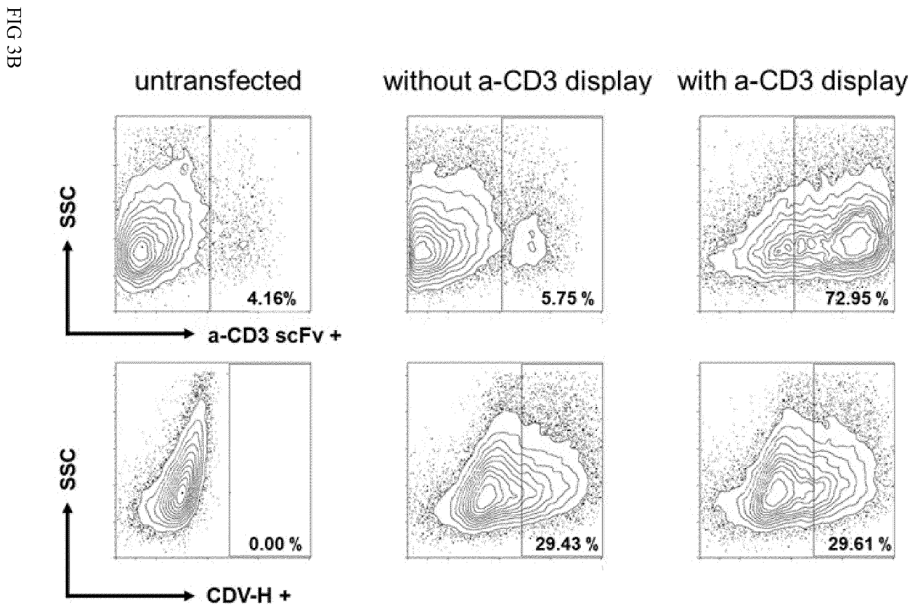

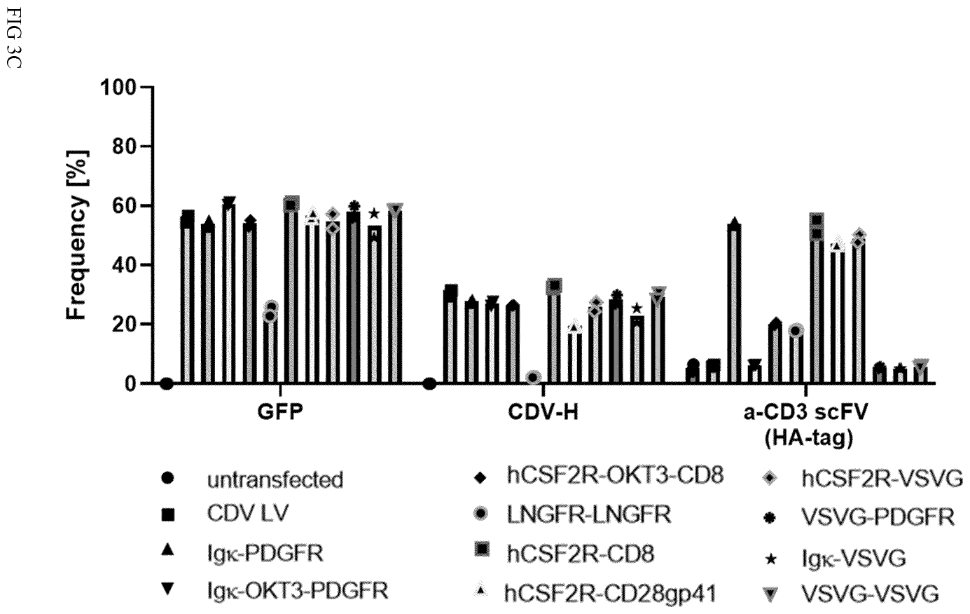

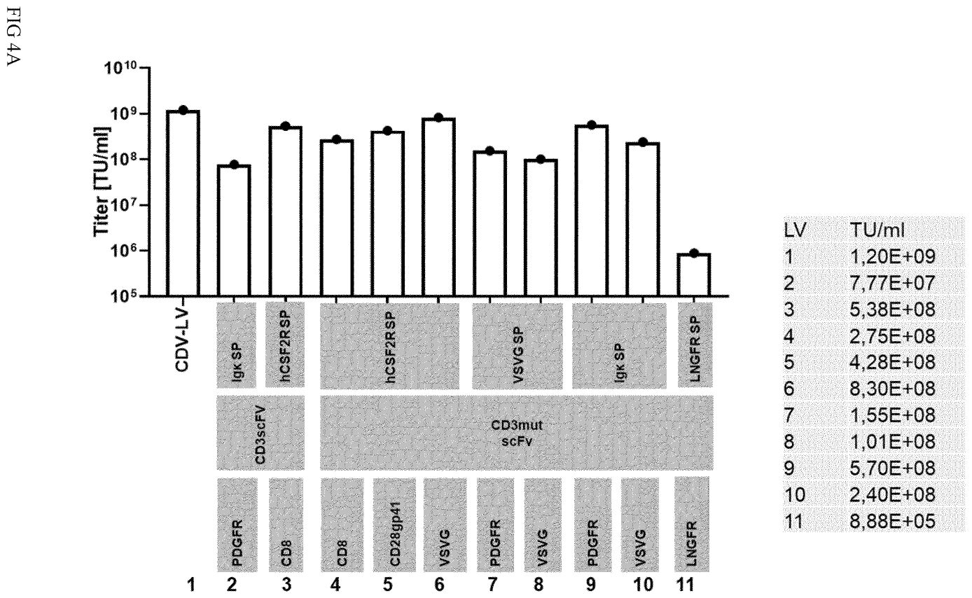

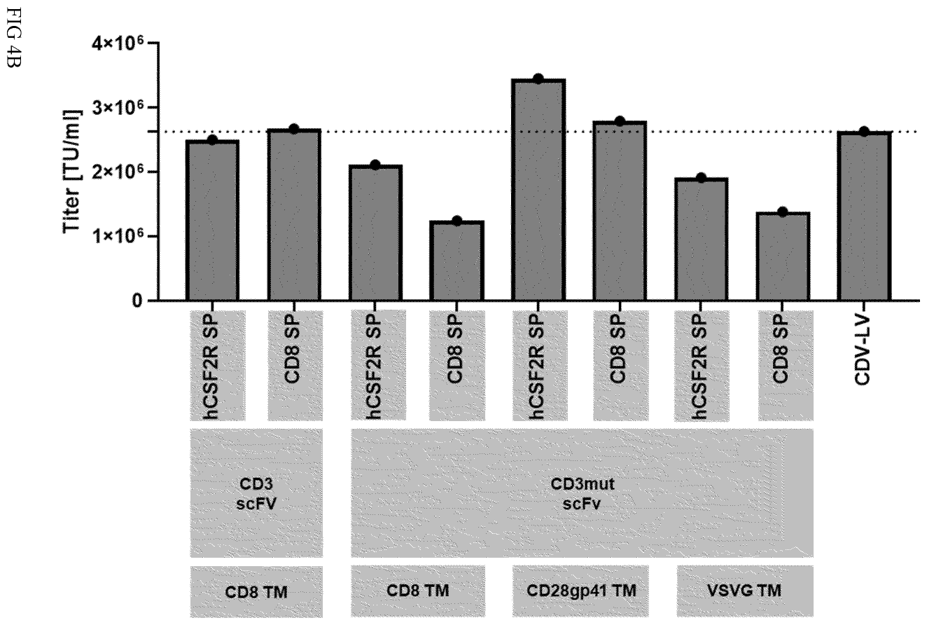

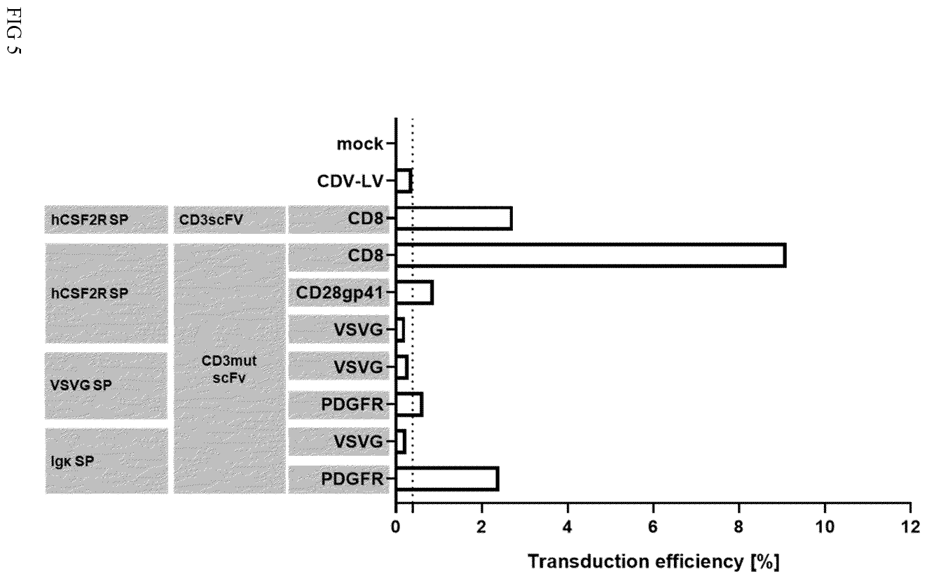

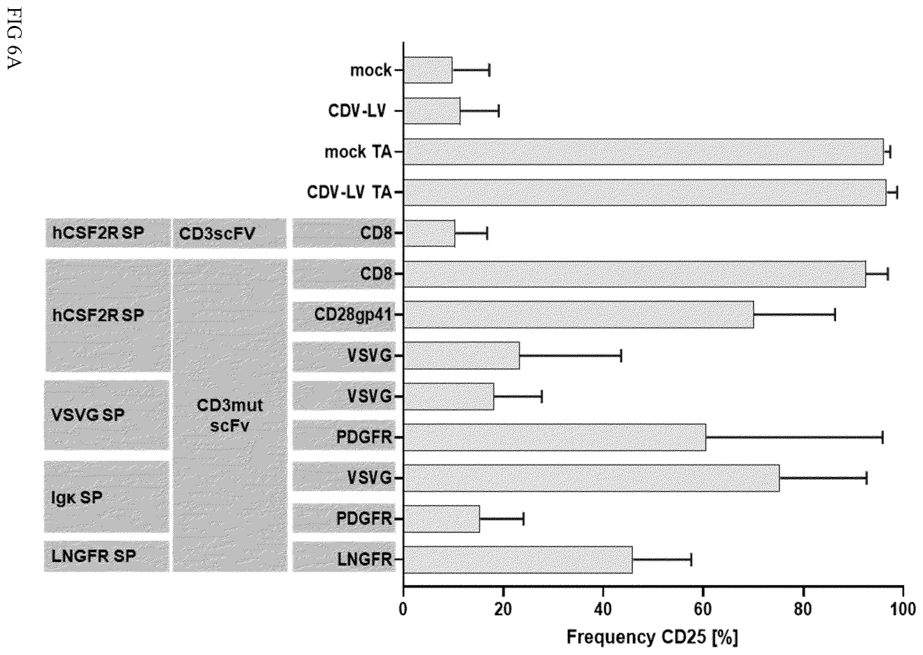

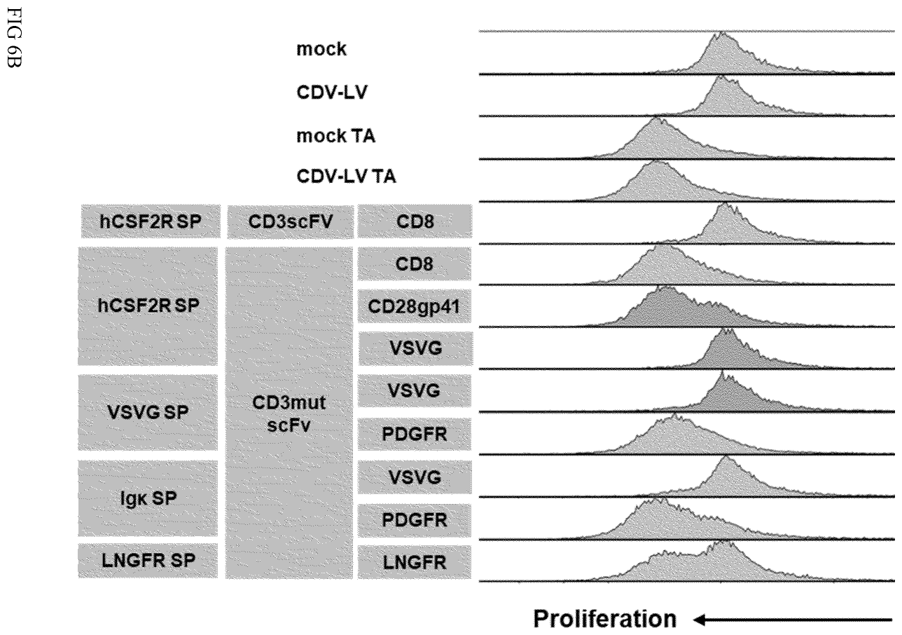

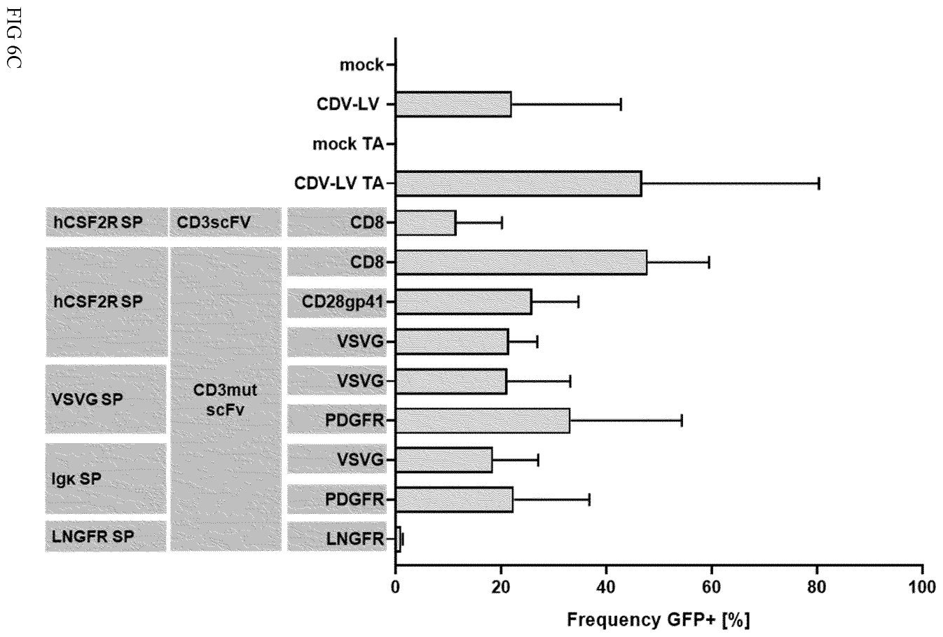

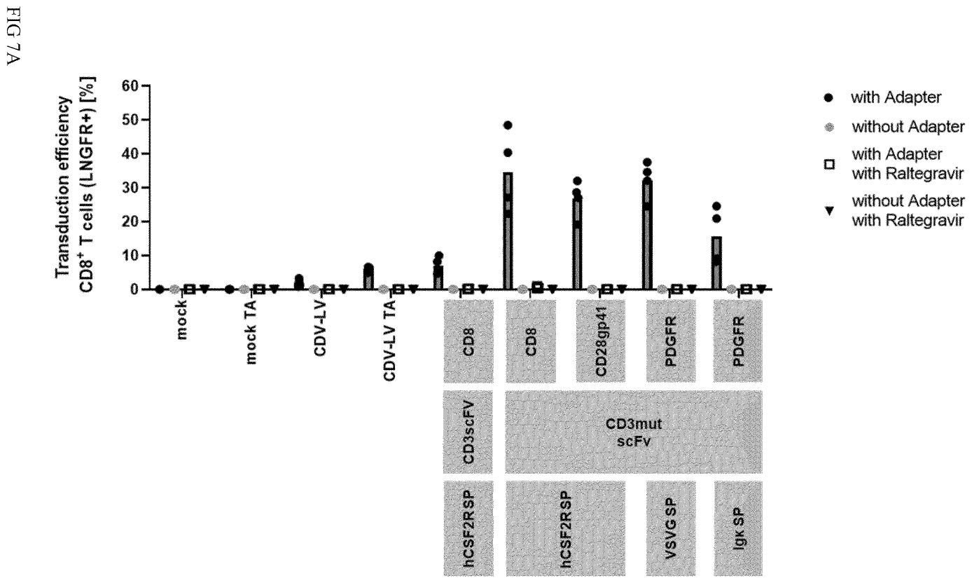

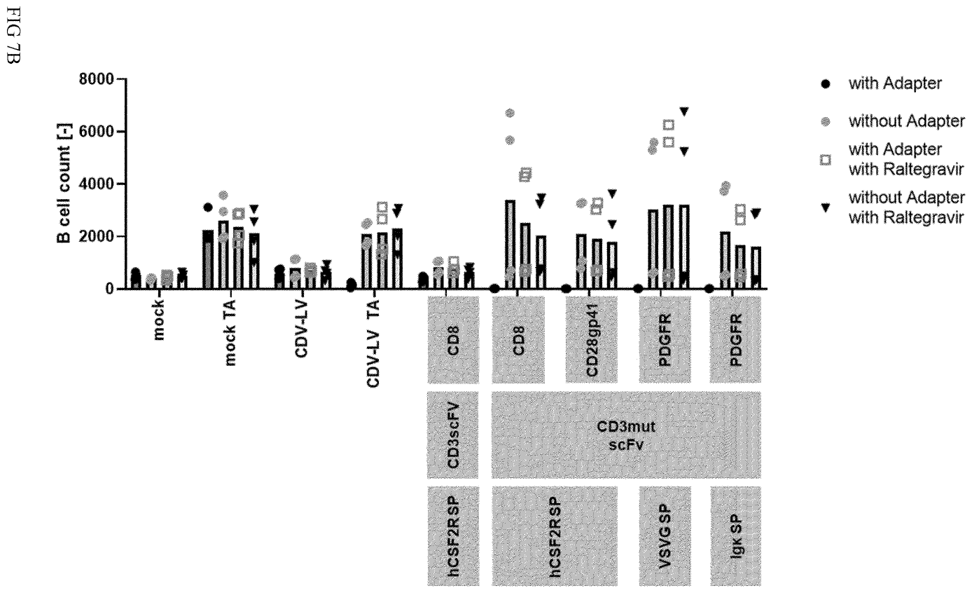

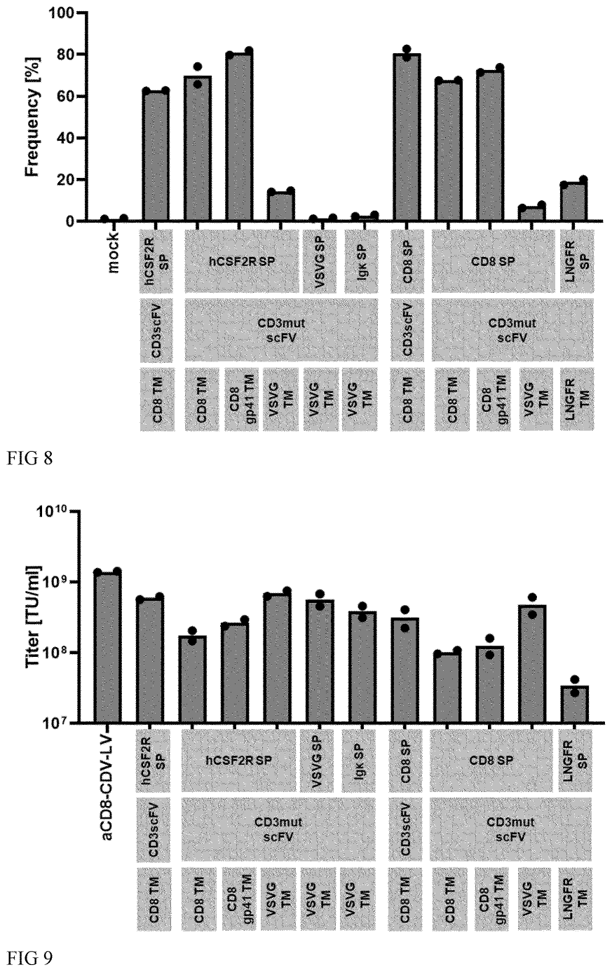

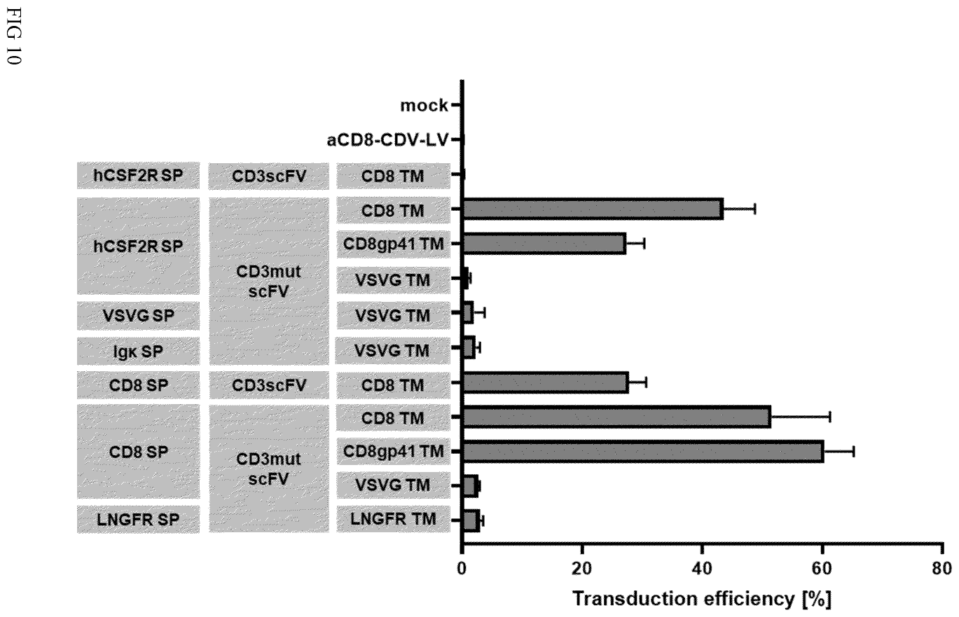

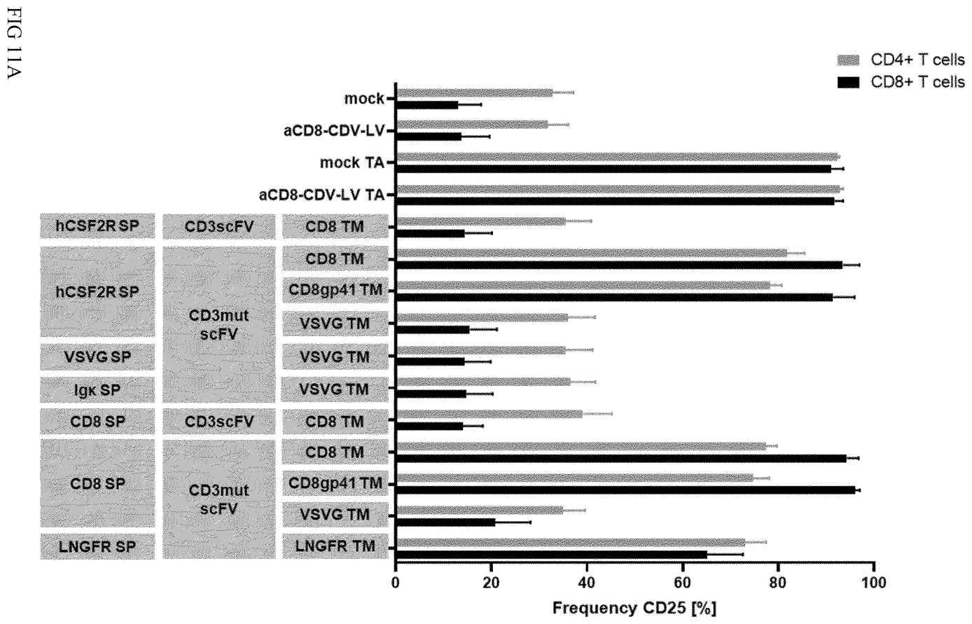

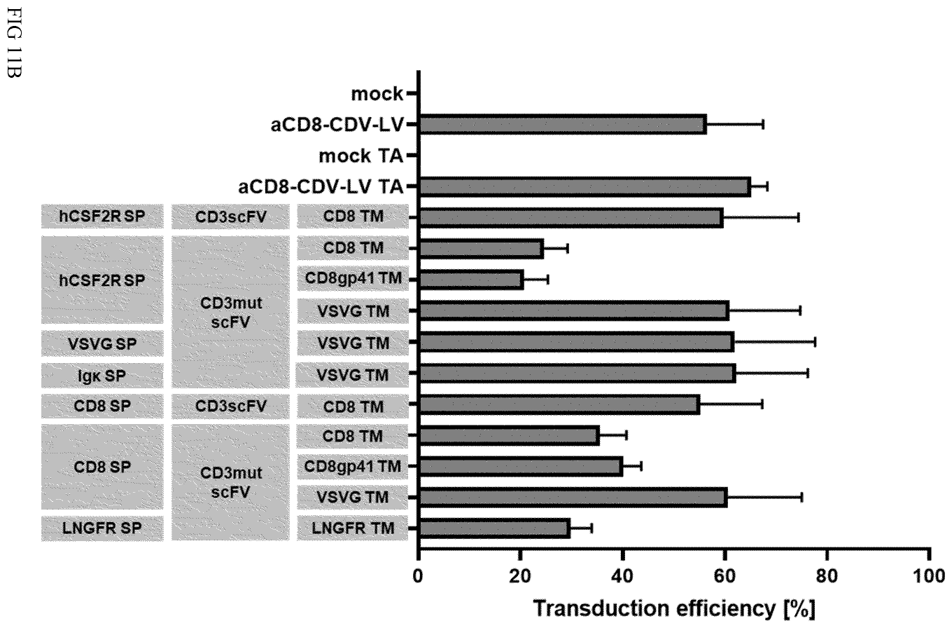

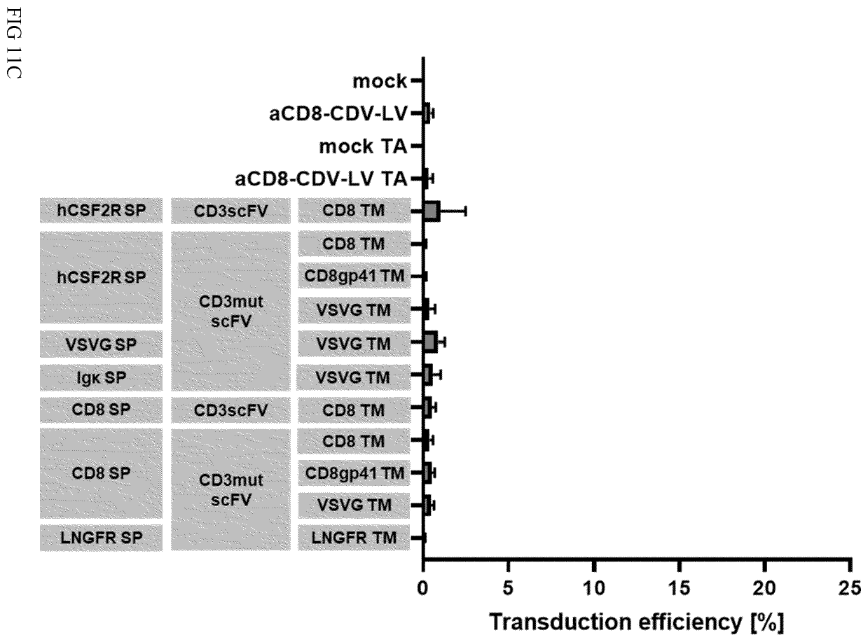

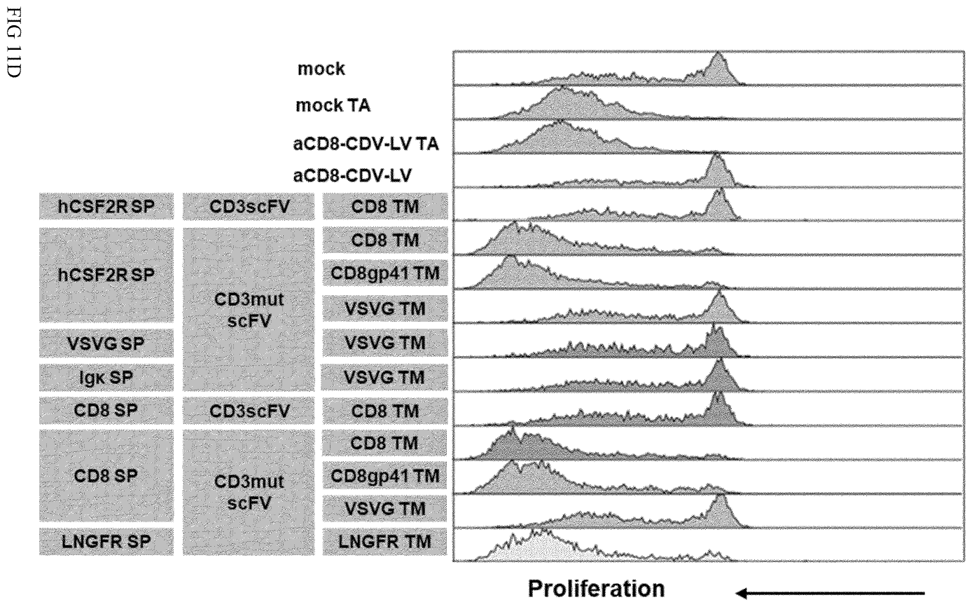

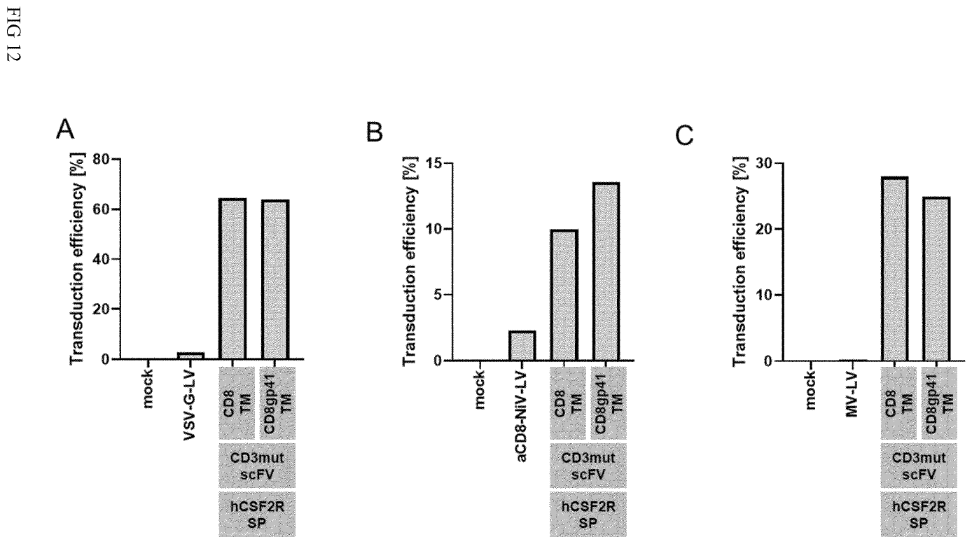

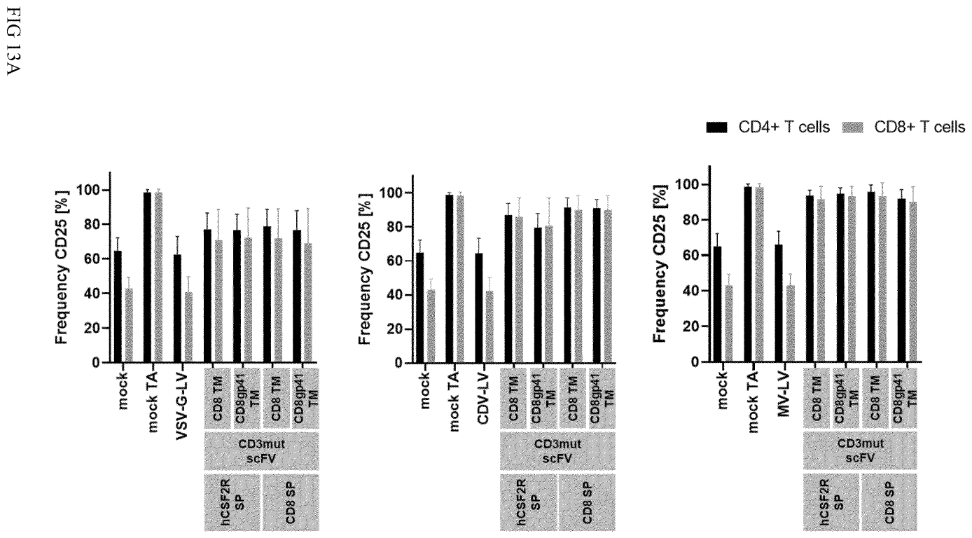

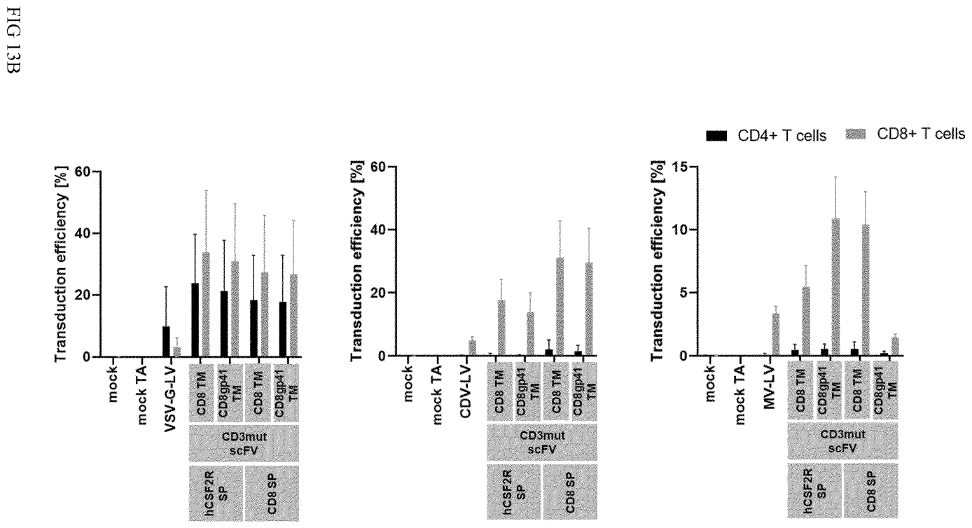

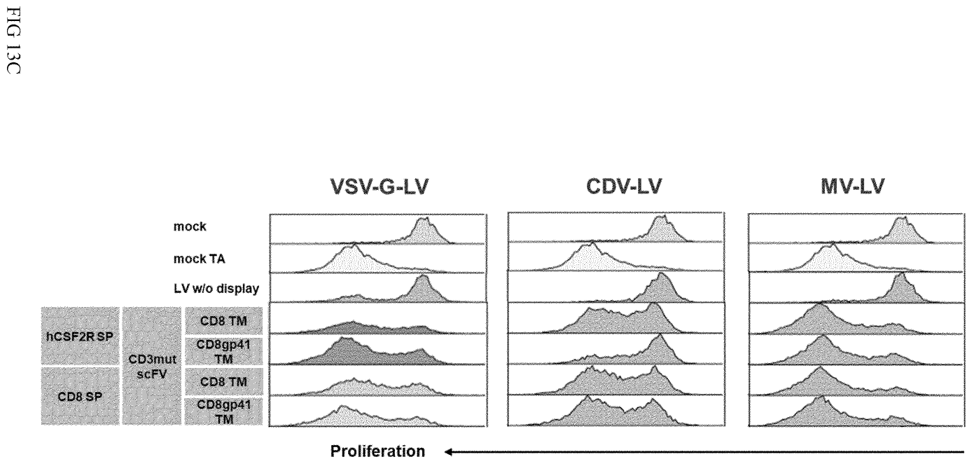

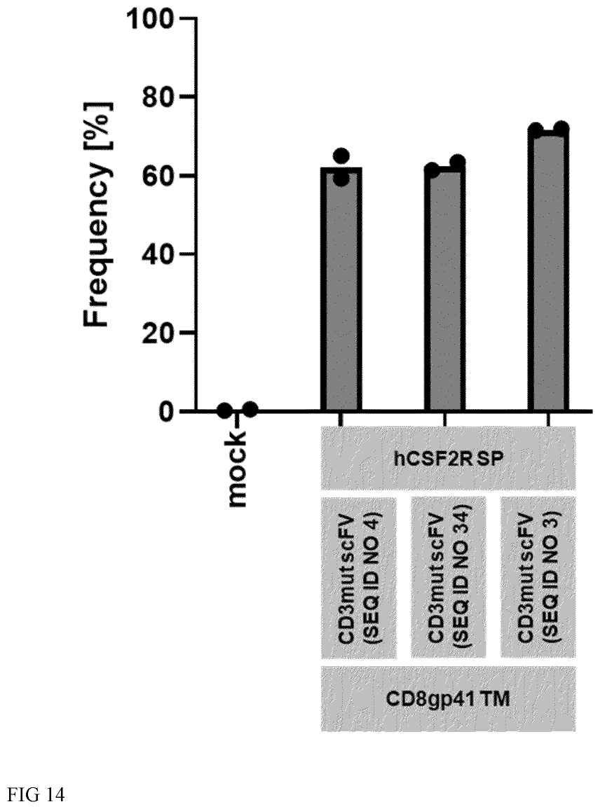

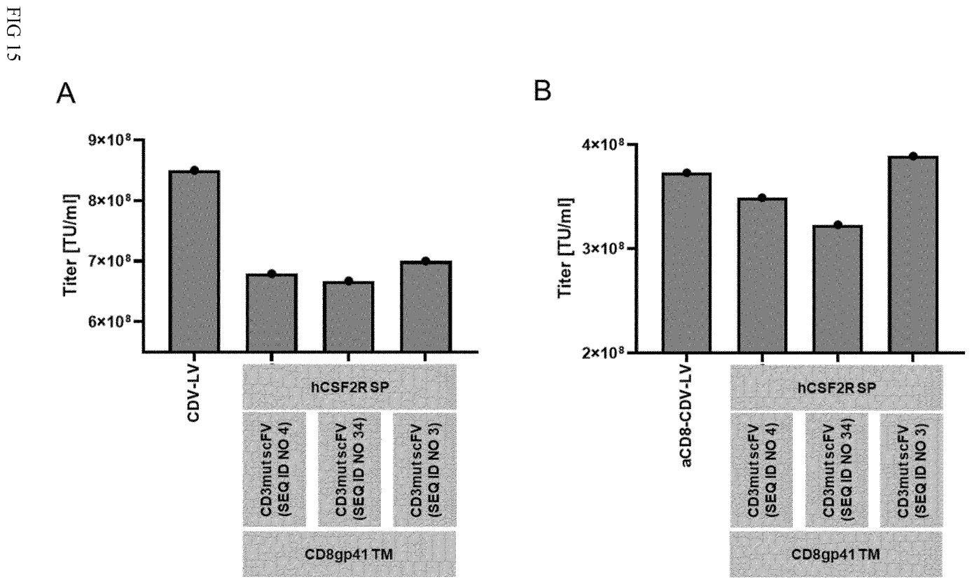

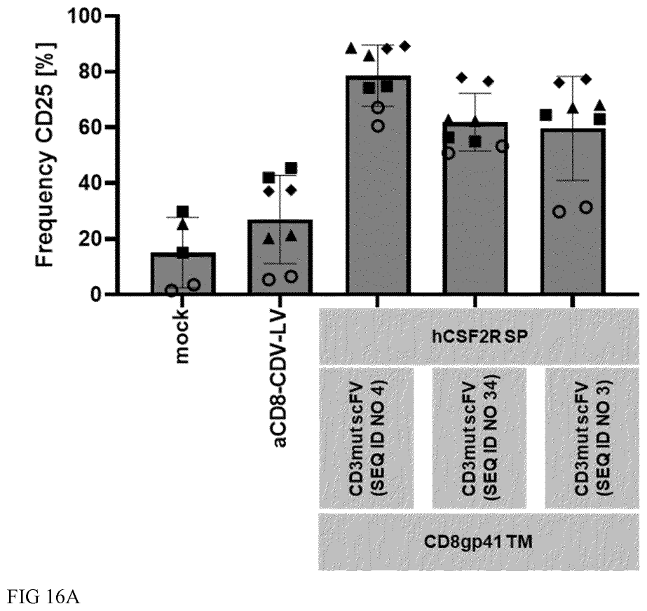

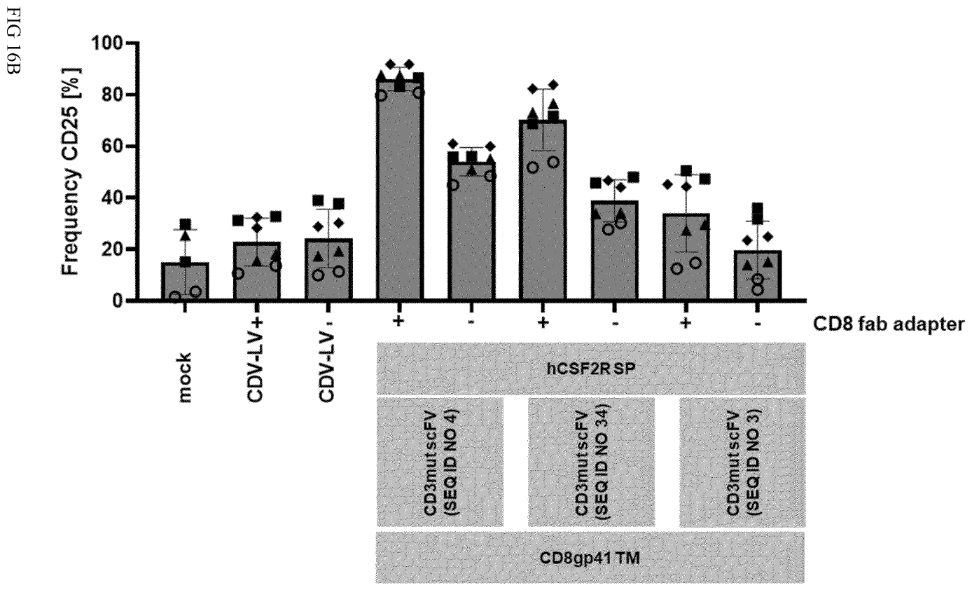

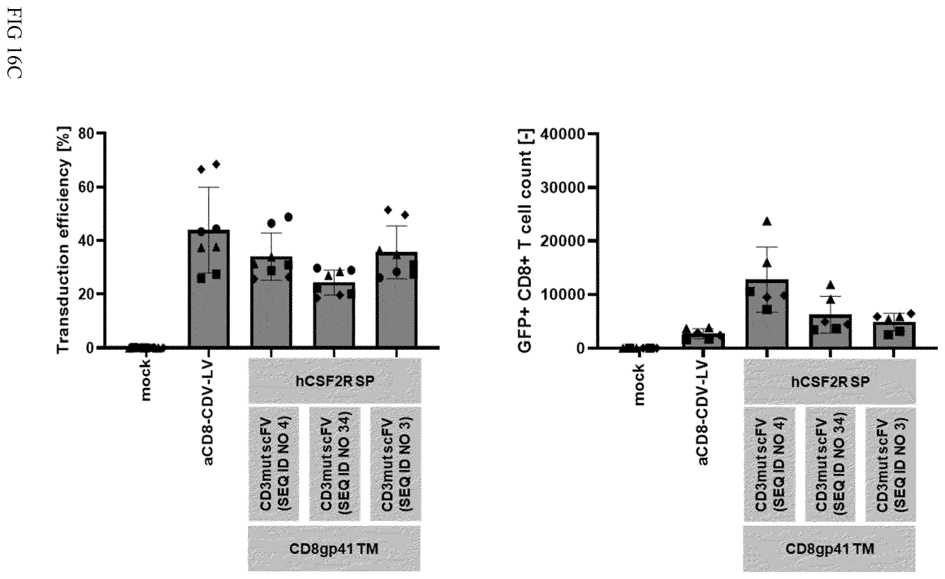

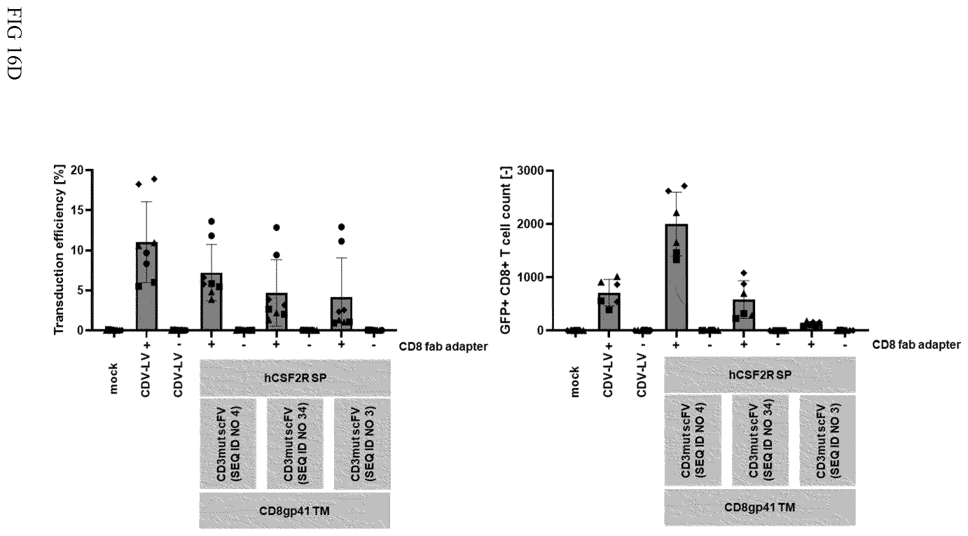

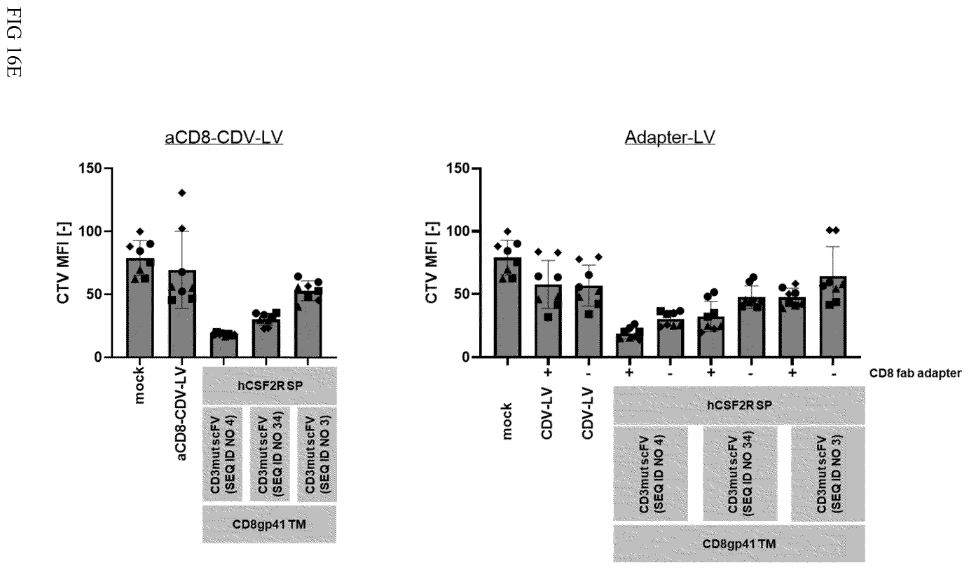

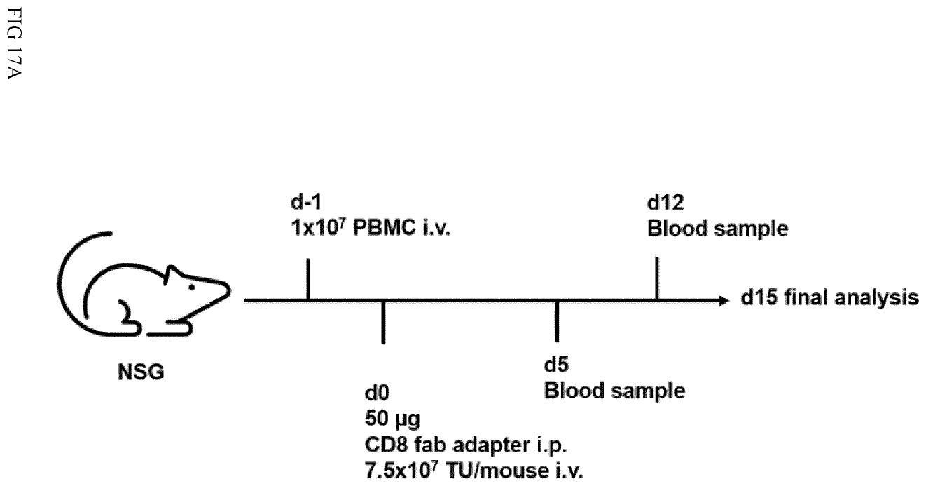

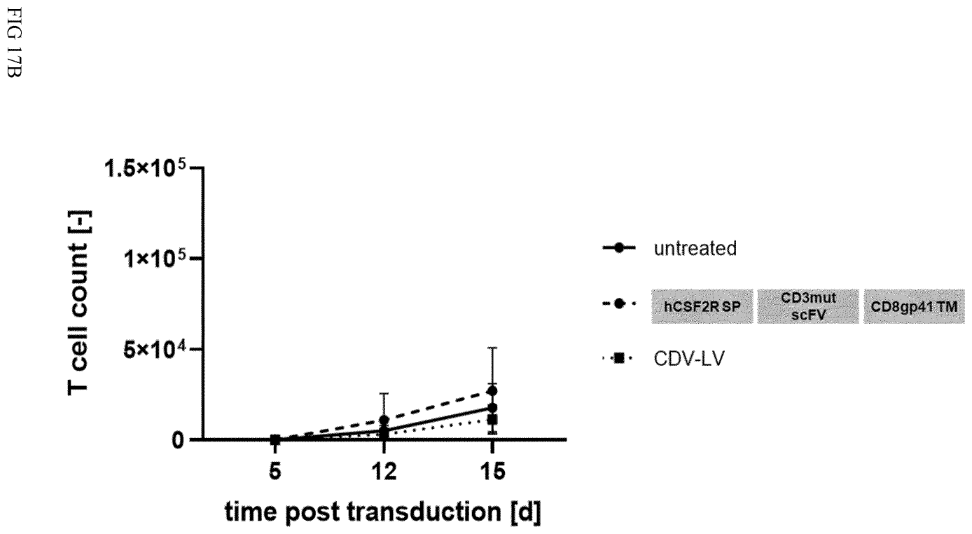

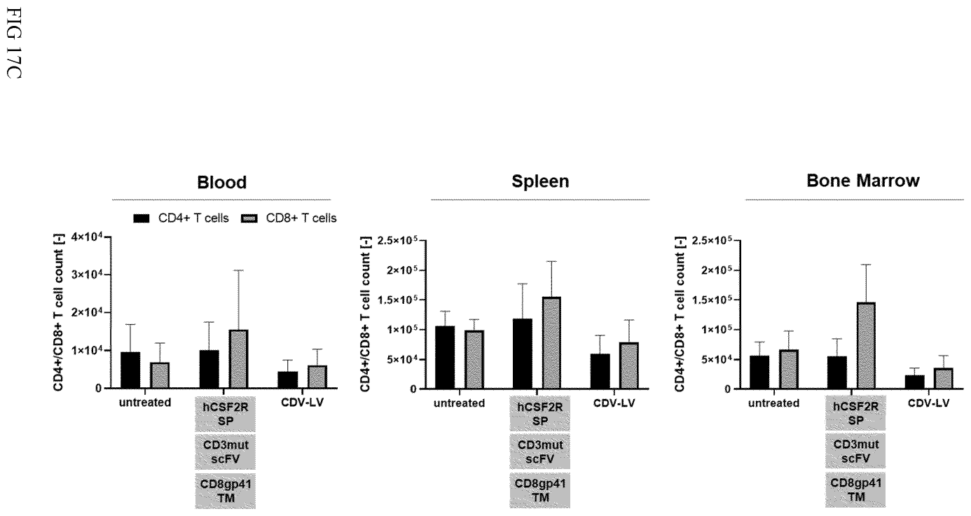

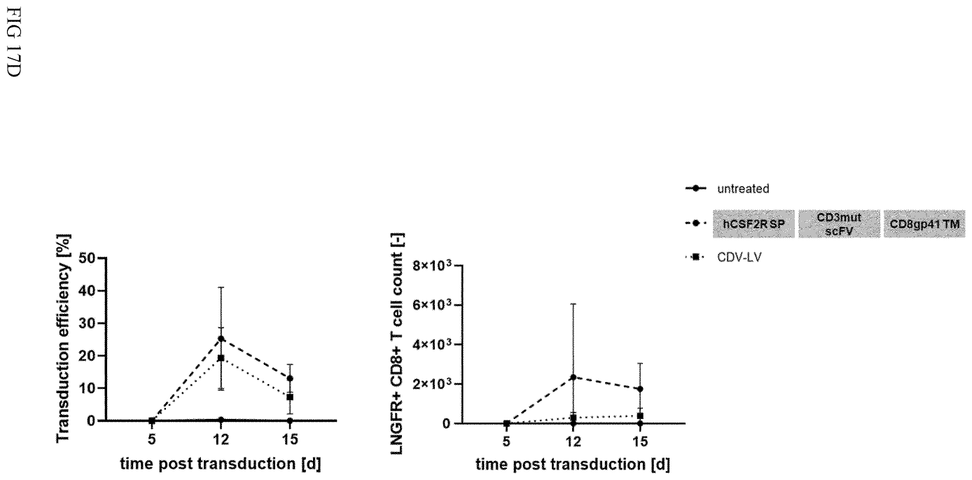

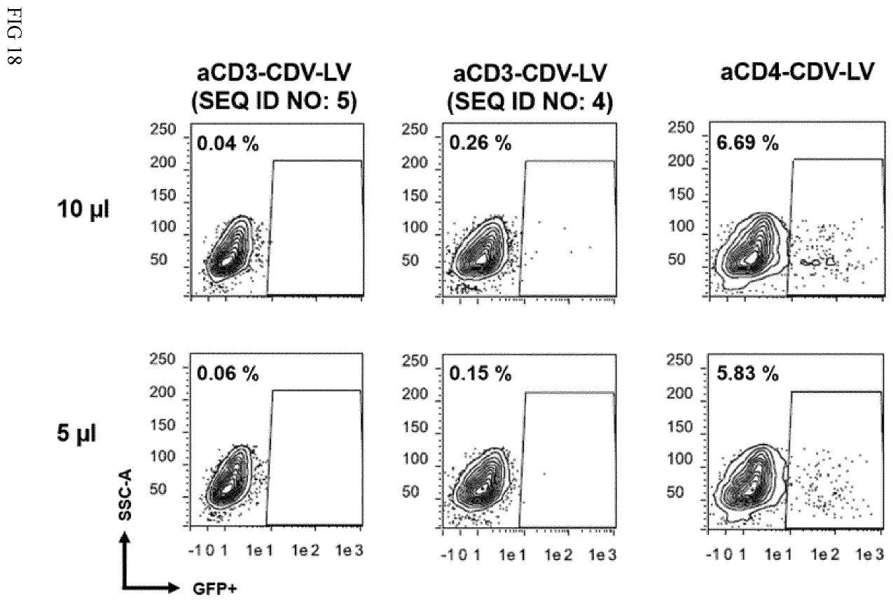

To enable simultaneous transduction and activation of T cells, an envelope protein was designed that confers T cell modulating capacity. This modulating protein can be incorporated in the envelope of pseudotyped retroviral particles. Suitable pseudotypes may be Canine distemper virus (CDV), Measles virus (MV), Nipah virus (NiV) and Vesicular stomatitis virus (FIG. 1A). For selective transduction the envelope glycoproteins of CDV, MV and NiV may be modified to ablate binding to their native receptors but bind to a desired antigen, e.g., CD4, CD8, or a tag, by fusion with an scFVs.

During the transduction process the retroviral particles bind via the modulating protein, that is comprising an a-CD3 scFV, to the TCR. This results in activation of the T cells, which can be observed in activation marker upregulation, enhanced metabolism and proliferation of the T cells. In contrast, binding of the envelope glycoproteins to their designated receptors will result in transduction of the T cells, characterized by integration and expression of the transgene, e.g. GFP or a CAR (FIG. 1B).

### Example 2: Cell Surface Expression of the Modulating Protein

Cell surface expression of the modulating proteins is crucial for successful incorporation into retroviral vector particles. To enable cell surface expression the a-CD3 scFV is expressed with a signaling peptide enabling translocation to the cell surface. A transmembrane and cytoplasmic domain ensures anchoring in the cellular membrane as well as successful incorporation into the retroviral particle (FIG. 2A). Surface expression was determined by transient transfection of HEK293T cells. For that, HEK293T cells were seeded in 6 wells with a density of 8×10⁵ cells/well one day before transfection. The HEK293T cells were transfected with the plasmids encoding the modulating protein or GFP as a positive control. Two days post transfection the cells were stained for expression of the a-CD3 scFV via a HA tag using the respective antibody (Miltenyi Biotec, Cat. No. 130-123-553), followed by flow cytometry to determine the ratio of HA positive cells (FIG. 2B, FIG. 8, FIG. 14). Results indicated that the surface expression of the modulating protein is influenced by the incorporated transmembrane domain (FIG. 2B). Modulating proteins containing CD8 transmembrane domains were expressed at higher levels compared to proteins with transmembrane domains derived from VSV-G or LNGFR (FIG. 8). Of note, surface expression of the modulating protein comprising a VSV-G transmembrane domain remained at low levels, independent of the comprised signal peptide. Vice versa, high surface expression of modulating proteins containing CD8 transmembrane domain remained high independent of the comprised signal peptide (FIG. 8). Interestingly, the surface expression was independent of the CD3 scFV sequence (FIG. 2B, FIG. 8, FIG. 14).

### Example 3: Generation of Anti-CD3-Displaying Retroviral Vectors

Pseudotyped retroviral vector particles specific for a tag of tagged polypeptide or specific for a target antigen expressed on a cell were generated by transient transfection of HEK293T cells. HEK293T cells that were seeded in T175 flasks in DMEM/10% FCS (Biowest, Cat. No. 12362; Biochrom, Cat. No. S0415) the day before were transfected with a plasmid encoding for the CDV or MV H protein or Nipah G, a plasmid encoding for the CDV, MV or Nipah F protein, or a plasmid encoding for the VSV G protein, a plasmid encoding for the modulating protein, a packaging plasmid encoding gag/pol/rev and a psi-positive transfer vector plasmid encoding the transgene, e.g. GFP or CD20CAR (FIG. 3A). The pseudotyped retroviral vector particles were harvested 48 h and 72 h post transfection. To remove cellular debris, the supernatant was collected, centrifuged for 10 min at 1000 rpm, followed by filtration through a 0.45 μm filter. To concentrate, the filtered supernatant was centrifuged through a 20% sucrose (Sigma Aldrich, Cat. No. 84097-250 g, 20% w/v in PBS) cushion for 24 h at 4° C. with 5350×g. The pelleted retroviral vectors were resuspended in 250 μl precooled PBS, aliquoted and stored at −80° C. for later use. To confirm sufficient transfection for production, the producer cells were stained after vector harvest for expression of the a-CD3 scFV and the CDV H protein. Subsequently, transfection efficiency was determined by quantification of the a-CD3 scFV, H and GFP expressing cells using flow cytometry (FIG. 3B, C).

### Example 4: Titration of Anti-CD3-Displaying Retroviral Vectors

Pseudotyped retroviral vector particles were titrated on SupT1 cells. Tag-specific LVs were titrated in the absence of the tagged polypeptide. Therefore, cell surface proteins were randomly labeled with LC-LC-biotin using by Biotin-LC-LC-NHS. SupT1 resuspended in 1 ml PBS were supplemented with Biotin-LC-LC-NHS followed by an incubation at 4° C. at constant mixing. After removing cell-free supernatant, cells were washed and seeded. For transduction SupT1 or biotinylated SupT1 were seeded with 2×10⁵ cells/well in 96-well in media (RPMI) without FCS. The LV particles were serially diluted in RPMI and added to the SupT1 cells. 96 h post transduction the transduction efficiency was determined by flow cytometry quantifying the ratio of GFP positive. The ratio of GFP positive cells, the dilution factor and the volume of retroviral applied is used to calculate the retroviral vector titer (i.e. transducing units per volume (TU/ml) (FIG. 4A-B, FIG. 9, FIG. 15A-B). CDV-pseudotyped retroviral vectors could be produced by HEK293T cells co-expressing the modulating proteins. Only, in presence of modulating proteins comprising a LNGFR transmembrane domain (SEQ ID: 32) resulted in a significant decrease in vector productivity. Importantly, the a-CD3 scFV did not influence the productivity (FIG. 15A-B).

### Example 5: Analysis of Incorporated Anti-CD3-scFV

Pseudotyped retroviral particles were immobilized via a N-terminal tag comprised in the modulating protein to confirm the incorporation of the a-CD3 scFV in functional retroviral particles. Therefore, a 96-well ELISA plate was coated with anti-tag antibodies and washed with PBS containing 0.05% Tween three times. After blocking with PBS containing 0.05% Tween with 2% BSA for two hours, GFP encoding retroviral vector particles diluted in PBS containing 2% BSA were added to the wells and incubated at room temperature for 1.5 hours. Unbound particles were removed by washing three times with PBS containing 2% BSA. Biotinylated SupT1 were prepared as described in Example 4 and added to the wells with a density of 2×10⁵ cells/well. For CD8-targeted retroviral vector particles or VSV-G pseudotyped retroviral vector particles, SupT1 were added with a density of 2×10⁵ cells/well. The transduction efficiency was determined by quantification of GFP-expressing cells using flow cytometry six to eight days after transduction (FIG. 5, FIG. 10, FIG. 12A-C). The data suggest, that the modulating protein was incorporated into functional retroviral vectors. The level of immobilization varied with the architecture of the modulating protein. A lower transduction efficiency suggests, that the level of incorporation of the modulating protein comprising LNGFR or VSV-G transmembrane domains is reduced compared to modulating proteins comprising PDGFR, CD28 or CD8 transmembrane domains (FIG. 5, FIG. 10). Importantly, the level of incorporation was influenced by the a-CD3 scFV sequence. Of note, incorporation of the modulating protein into VSV-G, MV or NiV pseudotyped retroviral particles was achieved (FIG. 12A-C).

### Examples 6: Transduction of Isolated Human T Cells with Anti-CD3 Displaying LV

PBMCs were isolated from buffy coat of healthy donors by density gradient centrifugation. Non-activated primary CD4+ and CD8+ T cells were isolated from the PBMC using the Pan T cell Isolation kit (Miltenyi Biotec, Cat. No. 130-096-535) and CD8+ T cells were isolated from the PBMC using the CD8 T cell Isolation kit (Miltenyi Biotec, Cat. No. 130-096-495) and stained with CellTrace™ Violet following the manufacturer's instructions (ThermoFisher, Cat. No. C34571). Pan T cells or CD8+ T cells were seeded with a density of 2×10⁵ cells/well in a 96-well plate and transduced with GFP encoding retroviral vector particles. The medium was replaced two days after transduction with fresh complete medium. T cells were stained two, five and/or eight days after transduction for expression of the activation marker CD25. Subsequently, T cell proliferation transduction efficiency and activation marker expression were determined by flow cytometry (FIG. 6, FIG. 11, FIG. 13, FIG. 16). Treatment of T cells with retroviral vectors displaying the modulating proteins resulted in upregulation of CD25, suggesting an induction of activation. The level of CD25 expression was dependent on the architecture of the modulating protein displayed by the respective retroviral vectors. Highest level of CD25 expression was achieved with retroviral vectors displaying modulating proteins comprising CD28, PDGFR and CD8 transmembrane domains (FIG. 6A, FIG. 11A). Treatment of PAN T cells with retroviral vectors displaying the modulating protein resulted in induction of CD25 expression on both CD4+ and CD8+ T cells (FIG. 11A). Importantly, the level of CD25 expression on T cells treated with modulating protein comprising a CD8 transmembrane domain and the a-CD3 scFV sequence of SEQ ID 4 was comparable to the level of CD25 expression of Transact™ activated T cells. Highest CD25 expression levels were achieved with modulating proteins comprising an a-CD3 scFV with SEQ ID 4 compared to modulating proteins comprising SEQ ID 3 or SEQ ID 34 (FIG. 16A-B). T cell activation was also achieved with VSV-G or MV-pseudotyped retroviral vectors displaying the modulating protein (FIG. 13A). Analysis of proliferation showed that induction of proliferation in absence of CD28 co-stimulation was achieved (FIG. 6B, FIG. 11D, FIG. 13C, FIG. 16E). Correlating with the CD25 expression levels, also the highest level of proliferation was achieved with retroviral vectors displaying modulating proteins comprising CD8 transmembrane domains and the a-CD3 scFV of SEQ ID 4 (FIG. 6B, 11D). Importantly, the highest level of proliferation was achieved with modulating proteins comprising SEQ ID 4 as compared to SEQ ID 3 and SEQ ID 34 (FIG. 16E). Proliferation was also induced by VSV-G or MV pseudotyped retroviral vectors displaying the modulating protein (FIG. 13C).

Retroviral vectors displaying the modulating protein efficiently transduced the non-activated T cells (FIG. 6C, FIG. 11B-C, FIG. 13B, FIG. 16C-D). Transduction efficiency was increased in presence of the modulating protein for VSV-G pseudotyped LV and adapter-specific retroviral vectors, resulting also in an increased number of transduced cells (FIG. 16C-D). Importantly, the transduction remained to be selective as no transduction of CD4+ T cells was observed upon transduction with CD8-specific retroviral vectors comprising the modulating protein (FIG. 11B-C).

### Examples 7: Transduction of Human PBMC with Anti-CD3 Displaying LV

PBMCs of healthy donors were isolated from buffy coat by density gradient centrifugation. The PBMC were seeded with 2.5×10⁵ cells/well in TexMACS™ medium supplemented with 12.5 ng/ml IL7 and 12.5 ng/ml IL15 in a 96-well plate. For respective samples, 1 μM of Raltegravir—an integrase inhibitor—was added one hour before transduction and maintained in the medium throughout the culture to exclude pseudo-transduction. The cells were incubated with CD8-specific fab adapter molecules for 30 min at 4° C., followed by transduction with CD20CAR encoding retroviral vector particles at a dose of 2.5 TU/cell. For designated samples TransAct™ was added together with the LV particles. The medium was replaced with fresh complete medium two days after transduction. Seven days post transduction, the transduction efficiency was analyzed by staining for the transduction marker LNGFR and subsequent flow cytometry analysis. In addition, viable B cells were quantified by flow cytometry after staining for CD19 (FIG. 7A-B). Efficient transduction only in presence of adapter and in absence of Raltegravir shows that a selective gene delivery with retroviral vectors was achieved. Depletion of B cells upon transduction suggests generation of functional CD20CAR T cells.

### Example 8: In Vivo Generation of CD20 CAR-T Cells with Adapter-Based Retroviral Vector Particles

PBMC of a healthy human Leukapheresis were isolated an incubated overnight in TexMACS™ medium supplemented with 12.5 ng/ml IL7 and 12.5 ng/ml IL15 before transplanted with 1×10⁷ PBMC/mouse into NSG mice. The next day, mice were left untreated or were i.p. injected with CD8-specific adapter molecules. Two- to three hours later, biotin-specific CDV-LV encoding a CD20CAR and the transduction marker LNGFR with or without displaying the modulating protein was i.v. injected with 7.5×10⁷ TU/mouse. Blood samples were taken 5 and 12 days post transduction via the vena facialis. 15 days post injection of retroviral vector particles the mice were sacrificed and blood, bone marrow and spleen tissue was harvested. The amount of human T cells and the level of CAR positive T cells was analyzed upon staining for respective markers and subsequent flow cytometry (FIG. 17A-D). The amount of human T cells in the mouse blood increased over time with a stronger increase in mice treated with retroviral vectors comprising the modulating protein (FIG. 17B). The endpoint analysis revealed a higher amount of CD8+ T cells upon transduction with retroviral vectors comprising the modulating protein compared to untreated mice or mice treated with the retroviral vector without display of the modulating protein (FIG. 17C). Most importantly the level of transduced T cells was increased with retroviral vectors displaying the modulating protein compared the control treated mice (FIG. 17D).

### Example 9: CD3 Targeted CDV-LV

GFP-encoding CDV-pseudotyped retroviral vector particles targeted against CD3 (aCD3-CDV-LV) were produced as described in Example 3. Subsequently, Jurkat cells were seeded with a density of 3×10⁵ cells/well in 48-well plate. aCD3-targeted retroviral vector particles were serially diluted in RMPI without FCS and added to the cells. After 96 hours, transduction efficiency was analyzed by quantification of GFP positive cells. CD4-targeted CDV-LV (aCD4-CDV-LV) was used as positive control. Interestingly, GFP expressing cells were not detected upon transduction with aCD3-CDV-LV (FIG. 18), suggesting that retargeting of CDV pseudotyped LV to CD3 results in generation of non-functional particles.

Sequence listing:
(VH of OKT3 mut)
SEQ ID NO: 1
QVQLVQSGGGVVQPGRSLRLSCKASGYTFTRYTMHWVRQAPGKGLEWIGYINPSRG
YTNYNQKVKDRFTISRDNSKNTAFLQMDSLRPEDIGVYFCARYYDDHYSLDYWGQ
GTPVTVSS
(VL of OKT3 mut)
SEQ ID NO: 2
DIQMTQSPSSLSASVGDRVTITCSASSSVSYMNWYQQTPGKAPKRWIYDTSKLASGV
PSRFSGSGSGTDYTFTISSLQPEDIATYYCQQWSSNPFTFGQGTKLQIT
(OKT3 mut; VL-VH)
SEQ ID NO: 3
DIQMTQSPSSLSASVGDRVTITCSASSSVSYMNWYQQTPGKAPKRWIYDTSKLASGV
PSRFSGSGSGTDYTFTISSLQPEDIATYYCQQWSSNPFTFGQGTKLQITGGGGSGGGGS
GGGGSQVQLVQSGGGVVQPGRSLRLSCKASGYTFTRYTMHWVRQAPGKGLEWIGYI
NPSRGYTNYNQKVKDRFTISRDNSKNTAFLQMDSLRPEDTGVYFCARYYDDHYSLD
YWGQGTPVTVSS
(OKT3 mut; VH-VL)):
SEQ ID NO: 4
QVQLVQSGGGVVQPGRSLRLSCKASGYTFTRYTMHWVRQAPGKGLEWIGYINPSRG
YTNYNQKVKDRFTISRDNSKNTAFLQMDSLRPEDTGVYFCARYYDDHYSLDYWGQ
GTPVTVSSGGGGSGGGGSGGGGSDIQMTQSPSSLSASVGDRVTITCSASSSVSYMNW
YQQTPGKAPKRWIYDTSKLASGVPSRFSGSGSGTDYTFTISSLQPEDIATYYCQQWSS
NPFTFGQGTKLQIT
(Okt3):
SEQ ID NO: 5
QVQLLESGAELARPGASVKMSCKASGYTFTRYTMHWVKQRPGQGLEWIGYINPSRG
YTNYNQKFKDKATLTTDKSSSTAYMQLSSLTSEDSAVYYCAGYYDDHYCLDYWGQ
GTLVTVSSGGGGSGGGGSGGGGSDIVMTQSPAIMSASPGEKVTMTCSASSSVSYMN
WYQQKSGTSPKRWIYDTSKLASGVPAHFRGSGSGTSYSLTISGMEAEDAATYYCQQ
WSSNPFTFGSGTKLEIK
(env incorporation signal (gp41)):
SEQ ID NO: 6
NRVRQGYS
(CD8 transmembrane domain):
SEQ ID NO: 7
IYIWAPLAGTCGVLLLSLVITLYCKRGRKK
(CD4 scFV VL):
SEQ ID NO: 8
DIVMTQTPLSLPVSLGDQASISCRSSQILVDSNENTYLHWYLQKPGQSPKLLIYKVSNR
VSGVPDRFSGSGSGTDFTLKISRVEAEDLGVYFCSQNTHVPPTFGGGTKLEIK
(CD4 scFV VH):
SEQ ID NO: 9
EVQLQQSGPELVKPGASVKMSCKASGYTFTNYVLHWVKQNSGQGLEWIGFIDPYND
GAQYNEKFKGKATLTSDKSSSTVYMEFSSLTSEDSAVYYCTREGGIGGFAYWGQGTL
VTVSA
(CD8 scFV VL):
SEQ ID NO: 10
DIVLTQSPPSLAVSLGQRATISCRASKSVSTSGYRFLHWYQQKPGQAPKLLIYLASNLE
SGVPARFSGSGSGTDFTLNIHPVEEEDAATYYCQHSREFPWTFGGGTKLEIK
(CD8 scFV VH):
SEQ ID NO: 11
EVQLQQSGAEVVRSGASVKLTCTVSGFNIKDHYMHWVKQRPEQGLEWIGWIDPENG
DTEYDPKFQGKATMTADISSNTAYLQLNSLTSEETAVYYCNALDSSGYGFAYWGQG
TLVTVSA
(CDV H 5840P):
SEQ ID NO: 12
MLSYQDKVGAFYKDNARANSSKLSLVTEEQGGRRPPYLLFVLLILLVGIMTLLAITGV
RFHQVSTSNMEFSRLLKEDMEKSEAVHHQVIDVLTPLFKIIGDEVGLQLPQKLNEIKQ
FILQKTNFFNPNREFDFRDLHWCINPPSKIKVNFTNYCDTIGIKKSIASAANPILLSALS
GGRGDIFPPYRCSGATTSVGRVFPLSVSLSMSLISKTSEITSMLTAISDGVYGKTYLLVP
DYIEGEFDTQKIRVFEIGFIKRWLNNMPLLQTTNYMVLPENSKAKVCTIAVGELTLAS
LCVDESTVLLYHDSNGSQDGILVVTLGIFGATPMDQVEEVIPVAHPSVEKIHITNHRGF
IKDSIATWMVPALVSEKQEEQKNCLESACQRKTYPMCNQTSWEPFGGGQLPSYGRLT
LPLDPSIDLQLNISFTYGPVILNGDGMDYYESPLSDSGWLTIPPRNGTVLGLINKASRG
DQFTVIPHVLTFAPRESSGNCYLPIQTSQIMDKDVLTESNLVVLPTQNFRYVIATYDIS
RGDHAIVYYVYDPIRTISYTHPFRLTTKGRPDFLRIECFVWDDDLWCHQFYRFEADIT
NSTTSVENLVRIRFSCNRSKP
(CDV F):
SEQ ID NO: 13
MHNKIPKGSKTPTPTQQDPPPQHSIKSAETKTSQARYNITSAQQSTRHGPRTSDRPVH
YIMNRTRSCKQASYRSDNIPAHGDHEGIIHHTPESVSQGVRSRFKRRQSNATSSGSQC
TWLVLWCIGVASLFLCSKAQIHWNNLSTIGIIGTDSVHYKIMTRPSHQYLVIKLMPNV
SLIDNCTKAELGEYEKLLSSVLEPINQSLTLMTKNVKPLQSVGSGRRQRRFAGVVLAG
AALGVATAAQITAGIALHQSNLNAQAIQSLRTSLEQSNKAIEEIREATQETIIAVQGVQ
DYVNNELVPAMQHMSCELVGQRLGLKLLRYYTELLSIFGPSLRDPISAEISIQALSYAL
GGEIHKILEKLGYSGNDMIAILESRGIKTKITHVDIPGKLIILSISYPTLSEVKGVIVHRLE
AVSYNIGSQEWYTTVPRYVATNGYLISNFDESSCVFVSESAICSQNSLYPMSPLLQQCI
RGDTSSCARTLVSGTMGNKFILSKGNIVANCASILCKCYSTSTIINQSPDKLLTFIASDT
CPLVEIDGVTIQVGGRQYPDMVYESKVALGPAISLERLDVGTNLGNALKKLDDAKILI
DSSNQILETVRRSSFNFGSLLSVPILICTALALLFLIYCCKRRYQQTLKQHIKVDPTFKP
DLTGTSKSYVRSL
(N-terminal signal Sequence F protein)
SEQ ID NO: 14
MHNKIPKGSKTPTPTQQDPPPQHSIKSAETKTSQARYNITSAQQSTRHGPRTSDRPVH
YIMNRTRSCKQASYRSDNIPAHGDHEGIIHHTPESVSQGVRSRFKRRQSNATSSGSQC
TWLVLWCIGVASLFLCSKA
(hCSF2R signal peptide):
SEQ ID NO: 15
MLLLVTSLLLCELPHPAFLLIP
(CD8 spacer):
SEQ ID NO: 16
TTTPAPRPPTPAPTIASQPLSLRPEACRPAAGGAVHTRGLDFACD
(hCD8 signal peptide):
SEQ ID NO: 17
MALPVTALLLPLALLLHAARP
(F wt measles):
SEQ ID NO: 18
MGLKVNVSAIFMAVLLTLQTPTGQIHWGNLSKIGVVGIGSASYKVMTRSSHQSLVIK
LMPNITLLNNCTRVEIAEYRRLLRTVLEPIRDALNAMTQNIRPVQSVASSRRHKRFAG
VVLAGAALGVATAAQITAGIALHQSMLNSQAIDNLRASLETTNQAIEAIRQAGQEMIL
AVQGVQDYINNELIPSMNQLSCDLIGQKLGLKLLRYYTEILSLFGPSLRDPISAEISIQA
LSYALGGDINKVLEKLGYSGGDLLGILESRGIKARITHVDTESYFIVLSIAYPTLSEIKG
VIVHRLEGVSYNIGSQEWYTTVPKYVATQGYLISNFDESSCTFMPEGTVCSQNALYP
MSPLLQECLRGSTKSCARTLVSGSFGNRFILSQGNLIANCASILCKCYTTGTIINQDPD
KILTYIAADHCPVVEVNGVTIQVGSRRYPDAVYLHRIDLGPPISLERLDVGTNLGNAIA
KLEDAKELLESSDQILRSMKGLSSTSIVYILIAVCLGGLIGIPALICCCRGR
CNKKGEQVGMSRPGLKPDLTGTSKSYVRSL
(cytoplasmic domain FcD30 measles):
SEQ ID NO: 19
CCRGR
(cytoplasmic domain FcD30 of Canine distemper virus strain 5840P):
SEQ ID NO: 20
ALLFLIYCCKRR
(cytoplasmic domain of HcD21 of Canine distemper virus strain 5840P):
SEQ ID NO: 21
MLSLVTEEQGGRRPPY
(cytoplasmic domain of HcD30 of Canine distemper virus strain 5840P):
SEQ ID NO: 22
MGRRPPY
(cytoplasmic domain of 4-1BB):
SEQ ID NO: 23
KRGRKKLLYIFKQPFMRPVQTTQEEDGCSCRFPEEEEGGCEL
(cytoplasmic domain of CD28):
SEQ ID NO: 24
RSKRSRLLHSDYMNMTPRRPGPTRKHYQPYAPPRDFAAYRS
(CD4 scFV complete):
SEQ ID NO: 25
DIVMTQTPLSLPVSLGDQASISCRSSQILVDSNENTYLHWYLQKPGQSPKLLIYKVSNR
VSGVPDRFSGSGSGTDFTLKISRVEAEDLGVYFCSQNTHVPPTFGGGTKLEIKGGGGS
GGGGSGGGGSEVQLQQSGPELVKPGASVKMSCKASGYTFTNYVLHWVKQNSGQGL
EWIGFIDPYNDGAQYNEKFKGKATLTSDKSSSTVYMEFSSLTSEDSAVYYCTREGGIG
GFAYWGQGTLVTVSA
(CD8 scFV complete):
SEQ ID NO: 26
DIVLTQSPPSLAVSLGQRATISCRASKSVSTSGYRFLHWYQQKPGQAPKLLIYLASNLE
SGVPARFSGSGSGTDFTLNIHPVEEEDAATYYCQHSREFPWTFGGGTKLEIKGGGGSG
GGGSGGGGSEVQLQQSGAEVVRSGASVKLTCTVSGFNIKDHYMHWVKQRPEQGLE
WIGWIDPENGDTEYDPKFQGKATMTADISSNTAYLQLNSLTSEETAVYYCNALDSSG
YGFAYWGQGTLVTVSA
(CD28 transmembrane domain)
SEQ ID NO: 27
FWVLVVVGGVLACYSLLVTVAFIIFWV
(PDGFR transmembrane domain)
SEQ ID NO: 28
AVGQDTQEVIVVPHSLPFKVVVISAILALVVLTIISLIILIMLWQKKPR
(Igk signal peptide)
SEQ ID NO: 29
METDTLLLWVLLLWVPGSTGD
(VSVG signal peptide)
SEQ ID NO: 30
MKCLLYLAFLFIGVNC
(LNGFR signal peptide)
SEQ ID NO: 31
MGAGATGRAMDGPRLLLLLLLGVSLGGA
(LNGFR transmembrane domain)
SEQ ID NO: 32
LIPVYCSILAAVVVGLVAYIAF
(VSVG transmembrane domain)
SEQ ID NO: 33
SSIASFFFIIGLII
(OKT3mut, VHmut-VL)
SEQ ID NO: 34
QVQLVQSGGGVVQPGRSLRLSCKASGYTFTRYTMHWVRQAPGKGLEWIGYINPSRG
YTNYNQKVKDRFTISRDNSKNTAFLQMDSLRPEDTGVYFCARYYDDHYCLDYWGQ
GTPVTVSS

### REFERENCES

Amirache F, Lévy C, Costa C, Mangeot P E, Torbett B E, Wang C X, Nègre D, Cosset F L, Verhoeyen E. Mystery solved: VSV-G-LVs do not allow efficient gene transfer into unstimulated T cells, B cells, and HSCs because they lack the LDL receptor. Blood. 2014 Feb. 27; 123 (9): 1422-4. doi: 10.1182/blood-2013-11-540641. Erratum in: Blood. 2014 Jun. 5; 123 (23): 3682. PMID: 24578496.
Arcangeli S, Bove C, Mezzanotte C, Camisa B, Falcone L, Manfredi F, Bezzecchi E, El Khoury R, Norata R, Sanvito F, Ponzoni M, Greco B, Moresco M A, Carrabba M G, Ciceri F, Bonini C, Bondanza A, Casucci M. CAR T cell manufacturing from naive/stem memory T lymphocytes enhances antitumor responses while curtailing cytokine release syndrome. J Clin Invest. 2022 Jun. 15; 132 (12): e150807. doi: 10.1172/JCI150807. PMID: 35503659; PMCID: PMC9197529.
Bah E S, Nace R A, Peng K W, Muñoz-Alía M Á, Russell S J. Retargeted and Stealth-Modified Oncolytic Measles Viruses for Systemic Cancer Therapy in Measles Immune Patients. Mol Cancer Ther. 2020 October; 19 (10): 2057-2067. doi: 10.1158/1535-7163.MCT-20-0134. Epub 2020 Aug. 26. PMID: 32847970; PMCID: PMC7875472.
Bender R R, Muth A, Schneider I C, Friedel T, Hartmann J, Plückthun A, Maisner A, Buchholz C J. Receptor-Targeted Nipah Virus Glycoproteins Improve Cell-Type Selective Gene Delivery and Reveal a Preference for Membrane-Proximal Cell Attachment. PLOS Pathog. 2016 Jun. 9; 12 (6): e1005641. doi: 10.1371/journal.ppat. 1005641. PMID: 27281338; PMCID: PMC4900575.
Dai J, Agosto L M, Baytop C, Yu J J, Pace M J, Liszewski M K, O'Doherty U. Human immunodeficiency virus integrates directly into naive resting CD4+ T cells but enters naive cells less efficiently than memory cells. J Virol. 2009 May; 83 (9): 4528-37. doi: 10.1128/JVI.01910-08. Epub 2009 Feb. 11. PMID: 19211752; PMCID: PMC2668451.
Derdak S V, Kueng H J, Leb V M, Neunkirchner A, Schmetterer K G, Bielek E, Majdic O, Knapp W, Seed B, Pickl W F. Direct stimulation of T lymphocytes by immunosomes: virus-like particles decorated with T cell receptor/CD3 ligands plus costimulatory molecules. Proc Natl Acad Sci USA. 2006 Aug. 29; 103 (35): 13144-9. doi: 10.1073/pnas.0602283103. Epub 2006 Aug. 21. PMID: 16924110; PMCID: PMC1559767.
Frank A M, Braun A H, Scheib L, Agarwal S, Schneider I C, Fusil F, Perian S, Sahin U, Thalheimer F B, Verhoeyen E, Buchholz C J. Combining T-cell-specific activation and in vivo gene delivery through CD3-targeted lentiviral vectors. Blood Adv. 2020 Nov. 24; 4 (22): 5702-5715. doi: 10.1182/bloodadvances.2020002229. PMID: 33216892; PMCID: PMC7686896.
Ghassemi S, Nunez-Cruz S, O'Connor R S, Fraietta J A, Patel P R, Scholler J, Barrett D M, Lundh S M, Davis M M, Bedoya F, Zhang C, Leferovich J, Lacey S F, Levine B L, Grupp S A, June C H, Melenhorst J J, Milone M C. Reducing Ex Vivo Culture Improves the Antileukemic Activity of Chimeric Antigen Receptor (CAR) T Cells. Cancer Immunol Res. 2018 September; 6 (9): 1100-1109. doi: 10.1158/2326-6066.CIR-17-0405. Epub 2018 Jul. 20. PMID: 30030295; PMCID: PMC8274631.
Huckaby J T, Landoni E, Jacobs T M, Savoldo B, Dotti G, Lai S K. Bispecific binder redirected lentiviral vector enables in vivo engineering of CAR-T cells. J Immunother Cancer. 2021 September; 9 (9): e002737. doi: 10.1136/jitc-2021-002737. PMID: 34518288; PMCID: PMC8438880.
Kalamasz D, Long S A, Taniguchi R, Buckner J H, Berenson R J, Bonyhadi M. Optimization of human T-cell expansion ex vivo using magnetic beads conjugated with anti-CD3 and Anti-CD28 antibodies. J Immunother. 2004 September-October; 27 (5): 405-18. doi: 10.1097/00002371-200409000-00010. PMID: 15314550.
Li Y, Kurlander R J. Comparison of anti-CD3 and anti-CD28-coated beads with soluble anti-CD3 for expanding human T cells: differing impact on CD8 T cell phenotype and responsiveness to restimulation. J Transl Med. 2010 Oct. 26; 8:104. doi: 10.1186/1479-5876-8-104. PMID: 20977748; PMCID: PMC2987859.
Liu Y P, Russell S P, Ayala-Breton C, Russell S J, Peng K W. Ablation of nectin4 binding compromises CD46 usage by a hybrid vesicular stomatitis virus/measles virus. J Virol. 2014 February; 88 (4): 2195-204. doi: 10.1128/JVI.02628-13. Epub 2013 Dec. 11. PMID: 24335299; PMCID: PMC3911550.
Massé N, Barrett T, Muller C P, Wild T F, Buckland R. Identification of a second major site for CD46 binding in the hemagglutinin protein from a laboratory strain of measles virus (MV): potential consequences for wild-type MV infection. J Virol. 2002 December; 76 (24): 13034-8. doi: 10.1128/jvi.76.24.13034-13038.2002. PMID: 12438629; PMCID: PMC136712.
Massé N, Ainouze M, Néel B, Wild T F, Buckland R, Langedijk J P. Measles virus (MV) hemagglutinin: evidence that attachment sites for MV receptors SLAM and CD46 overlap on the globular head. J Virol. 2004 September; 78 (17): 9051-63. doi: 10.1128/JVI.78.17.9051-9063.2004. PMID: 15308701; PMCID: PMC506930.
Maurice M, Verhoeyen E, Salmon P, Trono D, Russell S J, Cosset F L. Efficient gene transfer into human primary blood lymphocytes by surface-engineered lentiviral vectors that display a T cell-activating polypeptide. Blood. 2002 Apr. 1; 99 (7): 2342-50. doi: 10.1182/blood.v99.7.2342. PMID: 11895766.
von Messling V, Oezguen N, Zheng Q, Vongpunsawad S, Braun W, Cattaneo R. Nearby clusters of hemagglutinin residues sustain SLAM-dependent canine distemper virus entry in peripheral blood mononuclear cells. J Virol. 2005 May; 79 (9): 5857-62. doi: 10.1128/JVI.79.9.5857-5862.2005. PMID: 15827201; PMCID: PMC1082760.
Nakamura T, Peng K W, Harvey M, Greiner S, Lorimer I A, James C D, Russell S J. Rescue and propagation of fully retargeted oncolytic measles viruses. Nat Biotechnol. 2005 February; 23 (2): 209-14. doi: 10.1038/nbt1060. Epub 2005 Jan. 30. PMID: 15685166
Nakamura T, Peng K W, Vongpunsawad S, Harvey M, Mizuguchi H, Hayakawa T, Cattaneo R, Russell S J. Antibody-targeted cell fusion. Nat Biotechnol. 2004 March; 22 (3): 331-6. doi: 10.1038/nbt942. Epub 2004 Feb. 15. PMID: 14990955.
Patterson J B, Scheiflinger F, Manchester M, Yilma T, Oldstone M B. Structural and functional studies of the measles virus hemagglutinin: identification of a novel site required for CD46 interaction. Virology. 1999 Mar. 30; 256 (1): 142-51. doi: 10.1006/viro.1999.9644. PMID: 10087234.
Rasbach A, Abel T, Münch R C, Boller K, Schneider-Schaulies J, Buchholz C J. The receptor attachment function of measles virus hemagglutinin can be replaced with an autonomous protein that binds Her2/neu while maintaining its fusion-helper function. J Virol. 2013 June; 87 (11): 6246-56. doi: 10.1128/JVI.03298-12. Epub 2013 Mar. 27. PMID: 23536664; PMCID: PMC3648109.
Tahara M, Takeda M, Shirogane Y, Hashiguchi T, Ohno S, Yanagi Y. Measles virus infects both polarized epithelial and immune cells by using distinctive receptor-binding sites on its hemagglutinin. J Virol. 2008 May; 82 (9): 4630-7. doi: 10.1128/JVI.02691-07. Epub 2008 Feb. 20. PMID: 18287234; PMCID: PMC2293038.
Taube R, Zhu Q, Xu C, Diaz-Griffero F, Sui J, Kamau E, Dwyer M, Aird D, Marasco W A. Lentivirus display: stable expression of human antibodies on the surface of human cells and virus particles. PLOS One. 2008 Sep. 11; 3 (9): e3181. doi: 10.1371/journal.pone.0003181. PMID: 18784843; PMCID: PMC2527531.
Vongpunsawad S, Oezgun N, Braun W, Cattaneo R. Selectively receptor-blind measles viruses: Identification of residues necessary for SLAM- or CD46-induced fusion and their localization on a new hemagglutinin structural model. J Virol. 2004 January; 78 (1): 302-13. doi: 10.1128/jvi.78.1.302-313.2004. PMID: 14671112; PMCID: PMC303414.

1) A pseudotyped retroviral vector particle for activating and transducing T cells, wherein said retroviral vector particle comprises a modulating protein comprising a functional ectodomain comprising an antigen binding domain specific for the antigen CD3, wherein said antigen binding domain specific for the antigen CD3 comprises SEQ ID NO:1 and SEQ ID NO:2, wherein said modulating protein comprises:
I) said functional ectodomain
II) a transmembrane domain, wherein said transmembrane domain is the transmembrane domain of CD8, CD28 or PDGFR,
III) a cytoplasmic domain,
wherein said retroviral vector particle comprises at least one nucleic acid sequence encoding a transgene, and wherein said retroviral vector particle is a lentiviral or gammaretroviral vector particle.

2) The pseudotyped retroviral vector particle of claim 1, wherein said cytoplasmic domain comprises a viral incorporation motif.

3) The pseudotyped retroviral vector particle of claim 2, wherein said viral incorporation motif is the incorporation motif of gp41, preferentially comprising SEQ ID NO:6.

4) The pseudotyped retroviral vector particle of claims 1 to 3, wherein said retroviral vector particle comprises
A)
a) an envelope protein with antigen-binding and fusion activity, and wherein said envelope protein is protein G of a virus of the Rhabdoviridae family,
b) said modulating protein,
wherein said Rhabdoviridae virus is a virus of the vesiculovirus genus such as the Vesicular stomatitis virus (VSV), or
B)
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a T cell, and wherein said envelope protein is protein H of a virus of the Paramyxoviridae family,
b) an envelope protein with fusion activity of a virus of the Paramyxoviridae family,
c) said modulating protein,
wherein said target antigen expressed on the surface of said T cell is not CD3, and
wherein said Paramyxoviridae virus is a virus of the morbillivirus genus, or
C)
a) an envelope protein with antigen-binding activity, wherein said envelope protein is a recombinant protein that does not interact with at least one of its native receptors and is fused at its ectodomain to a polypeptide that specifically binds to a target antigen expressed on the surface of a T cell, and wherein said envelope protein is protein G of a virus of the Paramyxoviridae family,
b) an envelope protein with fusion activity of a virus of the Paramyxoviridae family,
c) said modulating protein,
wherein said target antigen expressed on the surface of said T cell is not CD3, and
wherein said Paramyxoviridae virus is a virus of the Henipavirus genus.

5) The pseudotyped retroviral vector particle of claim 6, wherein in said retroviral vector particle of B) said protein H of a virus of the Paramyxoviridae family lacks at least one part of the cytoplasmic region of said protein H, and wherein said envelope protein with fusion activity lacks at least one part of the cytoplasmic region of said envelope protein; or
wherein in said retroviral vector particle of C) said protein G of a virus of the Paramyxoviridae family lacks at least one part of the cytoplasmic region of said protein G, and wherein said envelope protein with fusion activity lacks at least one part of the cytoplasmic region of said envelope protein.

6) The pseudotyped retroviral vector particle of claim 4 or 5, wherein said polypeptide that specifically binds to said target antigen expressed on the surface of a T cell is specific for the antigen CD4 and comprises SEQ ID NO:8 and SEQ ID NO:9, or wherein said polypeptide that specifically binds to said target antigen expressed on the surface of a T cell is specific for the antigen CD8 and comprises SEQ ID NO:10 and SEQ ID NO:11.

7) The pseudotyped retroviral vector particle of claims 1 to 6 for use in treatment of a disease in a subject in need thereof.

8) A composition comprising
i) a pseudotyped retroviral vector particle for activating and transducing T cells comprising
a) an envelope protein with antigen-binding activity, wherein said envelope protein is a recombinant protein that does not interact with at least one of its native receptors and is fused at its ectodomain to a polypeptide that specifically binds to a tag of a tagged polypeptide,
b) an envelope protein with fusion activity,
c) a modulating protein comprising a functional ectodomain comprising an antigen binding domain specific for the antigen CD3, wherein said antigen binding domain specific for the antigen CD3 comprises SEQ ID NO:1 and SEQ ID NO:2, wherein said modulating protein comprises:
I) said functional ectodomain
II) a transmembrane domain, wherein said transmembrane domain is the transmembrane domain of CD8, CD28 or PDGFR,
III) a cytoplasmic domain,
ii) said tagged polypeptide, wherein said tagged polypeptide binds specifically to a target antigen expressed on the surface of a T cell, wherein said target antigen is not CD3, and
wherein said retroviral vector particle comprises at least a nucleic acid sequence encoding a transgene, and wherein said retroviral vector particle is a lentiviral or gammaretroviral vector particle.

9) The composition of claim 8, wherein said cytoplasmic domain comprises a viral incorporation motif.

10) The composition of claim 9, wherein said viral incorporation motif is the incorporation motif of gp41, preferentially comprising SEQ ID NO:6.

11) The composition of claims 8 to 10, wherein
A)
i) said pseudotyped retroviral vector particle comprises:
a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and wherein said envelope protein is protein H of a virus of the Paramyxoviridae family,
b) an envelope protein with fusion activity of a virus of the Paramyxoviridae family,
c) a modulating protein comprising a functional ectodomain comprising an antigen binding domain specific for the antigen CD3, wherein said antigen binding domain specific for the antigen CD3 comprises SEQ ID NO:1 and SEQ ID NO:2,
ii) said tagged polypeptide, wherein said tagged polypeptide binds specifically to a target antigen expressed on the surface of a T cell, wherein said target antigen is not CD3, and
wherein said retroviral vector particle comprises at least a nucleic acid sequence encoding a transgene,
wherein said Paramyxoviridae virus is a virus of the morbillivirus genus, and
wherein said retroviral vector particle is a lentiviral or gammaretroviral vector particle; or
B)
i) said pseudotyped retroviral vector particle comprises:
a) an envelope protein with antigen-binding activity, wherein said envelope protein is a recombinant protein that does not interact with at least one of its native receptors and is fused at its ectodomain to a polypeptide that specifically binds to a tag of a tagged polypeptide, and wherein said envelope protein is protein G of a virus from the Paramyxoviridae family,
b) an envelope protein with fusion activity of a virus of the Paramyxoviridae family,
c) a modulating protein comprising a functional ectodomain comprising an antigen binding domain specific for the antigen CD3, wherein said antigen binding domain specific for the antigen CD3 comprises SEQ ID NO:1 and SEQ ID NO:2,
ii) said tagged polypeptide, wherein said tagged polypeptide binds specifically to a target antigen expressed on the surface of a T cell, wherein said antigen is not CD3, and wherein said retroviral vector particle comprises at least a nucleic acid sequence encoding a transgene;
wherein said Paramyxoviridae virus is a virus of the Henipavirus genus, and
wherein said retroviral vector particle is a lentiviral or gammaretroviral vector particle.

12) The composition of claims 8 to 11, wherein said tagged polypeptide of A) or B) specific for said target antigen expressed on the surface of a T cell is specific for the antigen CD4 and comprises SEQ ID NO:8 and SEQ ID NO:9, or is specific for the antigen CD8 and comprises SEQ ID NO:10 and SEQ ID NO:11.

13) The composition of claims 8 to 12 for use in treatment of a disease in a subject in need thereof.
